SEQUENCE LISTING (3 sequences)

SEQ ID NO: 1 - protein, 15 aa, Artificial Sequence
  Synthetic deriviative of human (homo sapiens) atrial natriuretic peptide
MCHFGGRMDRISCYR

SEQ ID NO: 2 - protein, 15 aa, Artificial Sequence
  Synthetic deriviative of human (homo sapiens) atrial natriuretic peptide
MXHFGGRMDRISXYR

SEQ ID NO: 3 - protein, 13 aa, Artificial Sequence
  Synthetic deriviative of human (homo sapiens) atrial natriuretic peptide with N-terminal heptanoyl and C-terminal non-amino acid surrogate substituted keto-piperazine with guanidino group
CHFXGRXDRISCY

(19) United States
(12) Patent Application Publication
(10) Pub. No.: US 2016/0354443 A1
(43) Pub. Date: Dec. 8, 2016

(54) Replacement Therapy for Natriuretic Peptide Deficiencies

(71) Applicant: Palatin Technologies, Inc., Cranbury, NJ (US)

(72) Inventors: Carl Spana, West Harrison, NY (US); John H. Dodd, Spring Mills, PA (US); Trevor Hallam, Wayne, PA (US)

(21) Appl. No.: 15/214,674

(22) Filed: Jul. 20, 2016

Related U.S. Application Data
(63) Continuation of application No. PCT/US15/30324, filed on May 12, 2015.
(60) Provisional application No. 61/991,777, filed on May 12, 2014.

(51) Int. Cl. A61K 38/22; G01N 33/68; A61K 9/00

(52) U.S. Cl. CPC A61K 38/2242; A61K 9/0019; G01N 33/6893; G01N 2800/52

(57) ABSTRACT

Use and methods of use of natriuretic peptide mimetics which bind to and activate natriuretic peptide receptor A in patients with a defect, condition, syndrome, disease or mutation resulting in a functional active ANP₉₉₋₁₂₆ deficiency, for treatment or prophylaxis of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, including use of mimetics with a plurality of amino acid residues and at least one amino acid surrogate of formula I:

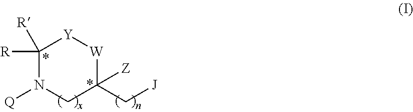

where R, R′, Q, Y, W, Z, J, x and n are as defined in the specification.

### CROSS-REFERENCE TO RELATED APPLICATION

This application is a continuation application of International Application No. PCT/US15/30324, published as International Publication No. WO 2015/175502, entitled “Replacement Therapy for Natriuretic Peptide Deficiencies”, filed on May 12, 2015, which in turns claims priority to and the benefit of the filing of U.S. Provisional Patent Application Ser. No. 61/991,777 entitled “Replacement Therapy for Natriuretic Peptide Deficiencies”, filed May 12, 2014, and the specification and claims thereof are incorporated herein by reference.

### BACKGROUND OF THE INVENTION

Field of the Invention (Technical Field)

The present invention relates to uses of natriuretic peptide mimetics, including those which include a plurality of amino acid residues and one or more ring-constrained amino acid surrogates and optionally one or more prosthetic groups, and formulations and methods of drug delivery relating thereto, for replacement therapy in patients with a deficiency in active endogenous natriuretic peptide, including endogenous atrial natriuretic peptide deficiencies.

Background Art

The natriuretic peptide system has been extensively explored since the identification of the human atrial natriuretic peptide (ANP) sequence and gene structure in 1984. ANP is sometimes also called ANF (atrial natriuretic factor), ANH (atrial natriuretic hormone) or atriopeptin. ANP is part of the natriuretic peptide system. In humans the ANP gene ultimately results in production of the most active form of ANP, ANP₉₉₋₁₂₆, which is also sometimes called α-ANP or ANP₁₋₂₈. Besides ANP₉₉₋₁₂₆, other forms of ANP include pro-ANP (also called ANP₁₋₁₂₆ or γ-ANP), N-terminal intervening peptide (NT-ANP), and urodilatin (ANP₉₅₋₁₂₆, a product of alternative processing of pro-ANP in kidney cells). The human ANP precursor peptide gene, which has 3 exons and 2 introns, encodes a 151-amino acid preprohormone which contains a 25-amino acid signal peptide sequence. During transport of the preprohormone across the endoplasmic membrane reticulum the signal peptide is cleaved, resulting in pro-ANP. The active forms of ANP result from proteolytic cleavage, primarily by corin, a specific converting enzyme for ANP. Corin cleavage results in NT-ANP and the most active form, ANP₉₉₋₁₂₆.

Besides ANP₉₉₋₁₂₆ and urodilatin, other active natriuretic peptides include BNP and CNP. ANP₉₉₋₁₂₆, urodilatin, BNP and CNP are each ring structures, with a homologous 17-amino acid loop formed by a cysteine-cysteine disulfide linkage. However, the prohormones for each of ANP, BNP and CNP are different, other than for the homologous loop, and the C- and N-terminal sequences outside the loop differ for each of ANP, BNP and CNP.

In general, the ANP structure, and ANP₉₉₋₁₂₆, is highly consersed during evolution. There is a difference of only one amino acid between human and rat ANP.

There are three known natriuretic peptide receptors, called natriuretic peptide receptors A, B and C (NPRA, NPRB and NPRC) or natriuretic peptide receptors 1, 2 and 3 (NPR1, NPR2 and NPR3). NPRA is also known as the guanylate-cyclase type A receptor. NPRA and NPRB are linked to guanylyl cyclases, while NPRC is a G-protein linked clearance receptor which may also have other regulatory functions, including inhibiting cyclic guanosine monophosphate (cGMP) or stimulating phosphoinositide hydrolysis. NPRA, NPRB and NPRC may be each referred to as a natriuretic peptide receptor (NPR). The active form of ANP binds to NPRA, which binding causes the conversion of guanosine triphosphase to cGMP, thereby raising intracellular cGMP. The active form of ANP also binds to NPRC, which results in clearance from circulation and potentially other regulatory actions. Activation of intracellular cGMP modulates the activity of a variety of regulatory proteins, with profound cardiovascular effects on vasodilation, blood pressure, natriuresis, modulating endothelial permability, and antagonizing the renin-angiotensin-aldosterone system and sympathetic nervous systems, among other effects.

It is known that there are certain defects, conditions, syndromes, diseases and mutations that may result in less than optimal or desirable levels of endogenous active natriuretic peptides, including active ANP₉₉₋₁₂₆. For example, it has been reported generally that human hypertension can be characterized by a lack of activation of active ANP₉₉₋₁₂₆. Macheret, F.; Heublein D. et al.: “Human hypertension is characterized by a lack of activation of the antihypertensive cardiac hormones ANP and BNP,” J Amer Coll Card 60:1558-65 (2012). Corin, a cardiac serine protease, converts pro-ANP to biologically active ANP by proteolytic cleavage. Yan, W.; Wu, F. et al.: “Corin, a transmembrane cardiac serine protease, acts as a pro-atrial natriuretic peptide-converting enzyme,” Proc Natl Acad Sc. USA 97:8525-8529 (2000). A deficit or deficiency in active ANP₉₉₋₁₂₆ may result from mutations in the gene coding for human corin. Wang, W.; Liao, X. et al.: “Corin variant associated with hypertension and cardiac hypertrophy exhibits impaired zymogen activation and natriuretic peptide processing activity,” Circ Res 103:502-508 (2008). Gene variants resulting in an alteration of the ANP primary sequence and henced altered ANP₉₉₋₁₂₆ can also result in increased susceptibility to acute coronary syndrome and unfavorable prognosis of coronary artery disease. Barbato, E.; Bartunek, J. et al.: “Influence of rs5065 atrial natriuretic peptide gene variant on coronary artery disease,” J Am Coll Cardiol 59:1763-1770 (2012).

Human ANP and BNP, including human BNP and ANP made by recombinant means, have been made and clinically used. Human recombinant BNP is approved and sold in the United States as nesiritide under the trade name Natrecor®. Human recombinant ANP is approved and sold in Japan and other countries as carperitide. However, both nesiritide and carperitide have very short circulation half-lives substantially similar to endogenously-produced active ANP and BNP, and thus are administered only by intravenous injection.

There are no reports of studies of treatment of cardiovascular disease by use of active natriuretic peptide, including ANP or mimetics thereof, in patients with defects, conditions, syndromes, diseases or mutations resulting in less than optimal or desirable levels of active endogenous natriuretic peptides, including active ANP₉₉₋₁₂₆. Ideally mimetics of endogenous active natriuretic peptides, including mimetics of active ANP, would have one or more of increased resistence to enzymatic degradation, increased circulation half life or mean residence time, increased bioavailability, increased efficacy, prolonged duration of effect and combinations of the foregoing compared to endogenous natriuretic peptides, including endogenous human active ANP. There is a substantial need for products with improved characteristics, including improved potency, half-life, modes of administration, bioavailability or prolonged duration of effect, which products are effective for treatment of cardiovascular disease by use of active natriuretic peptides, including active ANP or mimetics thereof, in patients with defects, conditions, syndromes, diseases or mutations resulting in less than optimal or desirable levels of active natriuretic peptides, including active ANP₉₉₋₁₂₆. It is against this background that the current invention was made.

### BRIEF SUMMARY OF THE INVENTION

In one aspect the invention provides a method of prophylaxis or treatment of disease, including but not limited to renal disease and cardiovascular disease, in patients with a defect, condition, syndrome, disease or mutation resulting in a functional active natriruretic peptide deficiency, by administration of a pharmaceutically effective amount of a natriuretic peptide mimetic which binds to an NPR, including at least one of NPRA and NPRB.

In another aspect the invention provides a method of prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active natriuretic peptide deficiency, by administration of a pharmaceutically effective amount of a natriuretic peptide mimetic which binds to an NPR, such as NPRA.

In another aspect the invention provides a method of prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, by administration of a pharmaceutically effective amount of a mimetic which binds to an NPR, such as NPRA.

In another aspect, the invention provides a method of prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, by administration of a pharmaceutically effective amount of a natriuretic peptide mimetic which binds to NPRA, wherein such mimetic includes a plurality of amino acid residues and at least one amino acid surrogate of the general formula I:

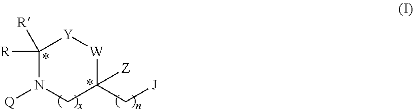

where R and R′ are each independently H or a natural or unnatural amino acid side chain moiety or derivative of an amino acid side chain moiety; x is 1 or 2; Y is CH₂ or C═O; W is CH₂, NH or NR′″; Z is H or CH₃; n is 0, 1 or 2; J is —C(═O)— unless the surrogate is at the C-terminus position of the mimetic, in which case J is —H, —OH, —C(═O)—OH, —C(═O)—NH₂ or a C-terminus capping group; Q is a bond unless the surrogate is at the N-terminus position of the mimetic, in which case Q is —H or an amine capping group; R′″ is an acyl, a C₁ to C₁₇ linear or branched alkyl chain, a C₂ to C₁₉ linear or branched alkyl acyl chain, a C₁ to C₁₇ linear or branched omega amino aliphatic, or a C₁ to C₁₇ linear or branched omega amino aliphatic acyl; optionally at least one prosthetic group covalently bonded to a reactive group in a side chain of at least one of the amino acid residues, to an amine capping group where the surrogate is at the N-terminus position of the mimetic, or to a C-terminus capping group where the surrogate is at the C-terminus position of the mimetic; and the carbon atoms marked with an asterisk can have any possible stereochemical configuration. The plurality of amino acid residues may include any amino acid residue selected from the group consisting of natural or unnatural α-amino acids, β-amino acids, α,α-disubstituted amino acids and N-substituted amino acids, including all (R) or (S) configurations of any of the foregoing.

The mimetic may be a cyclic mimetic, cyclized by a bond between side chains of two amino acid residues, between an amino acid residue side chain and an R or R′ group of an amino acid surrogate, between R or R′ groups of two amino acid surrogates, between a terminal group of the mimetic and an amino acid residue side chain, or between a terminal group of the mimetic and an R or R′ group of an amino acid surrogate. Preferable the two amino acid residues forming a bond between the side chains thereof are separated by between about eight and ten amino acid residues and optionally zero, one or two amino acid surrogates.

The prosthetic group(s) may include polymeric groups comprising repeat units including one or more carbon and hydrogen atoms, and optionally other atoms, including oxygen. Such polymeric groups are preferably water-soluble polymers, and are preferably poly(alkylene oxide), poly(vinyl pyrrolidone), poly(vinyl alcohol), polyoxazoline or poly(acryloylmorpholine). A preferred poly(alkylene oxide) is poly(ethylene glycol) (PEG), optionally derivatized with a linking group.

In one aspect, J is a C-terminus capping group selected from
—(CH₂)ₘ—OH,
—C(═O)—(CH₂)ₘ—N(v₁)(v₂),
—C(═O)—O—(CH₂)ₘ—CH₃,
—O—(CH₂)ₘ—CH₃,
—O—(CH₂)ₘ—N(v₁)(v₂),
—O—(CH₂)ₘ—OH,
—C(═O)—NH—(CH₂)ₘ—S(v₁),
—C(═O)—NH—(CH₂)ₘ—CH₃,
—C(═O)—NH—(CH₂)ₘ—N(v₁)(v₂),
—C(═O)—N—((CH₂)ₘ—N(v₁)(v₂))₂,
—C(═O)—NH—CH(—C(═O)—OH)—(CH₂)ₘ—N(v₁)(v₂),
—C(═O)—NH—(CH₂)ₘ—NH—C(═O)—CH(N(v₁)(v₂))((CH₂)ₘ—N(v₁)(v₂)), or
—C(═O)—NH—CH(—C(═O)—N(O(v₂))—(CH₂)ₘ—N(v₁)(v₂);
   including all (R) or (S) configurations of the foregoing, where v₁ and v₂ are each independently H or a C₁ to C₁, linear or branched alkyl chain and m is in each instance independently 0 to 17.

In another aspect where the amino acid surrogate is at the C-terminus position of the mimetic, J is a C-terminus capping group consisting of an omega amino aliphatic, terminal aryl or aralkyl group or any single natural or unnatural α-amino acid, β-amino acid, α,α-disubstituted amino acid or N-substituted amino acid, including all (R) or (S) configurations of an α,α-disubstituted amino acid where the substituents are different, optionally in combination with a C-terminus capping group as defined above.

In another aspect, Q is an amine capping group selected from
—(CH₂)ₘ—N(v₃)(v₄),
—(CH₂)ₘ—CH₃,
—(CH₂)ₘ—O(v₃),
—(CH₂)ₘ—C(═O)-(v₃),
—(CH₂)ₘ—C(═O)—O-(v₃),
—(CH₂)ₘ—S(v₃),
—C(═O)—(CH₂)ₘ—CH₃,
—C(═O)—(CH₂)ₘ—N(v₃)(v₄),
—C(═O)—(CH₂)ₘ—C(═O)-(v₃),
—C(═O)—(CH₂)ₘ—O(v₃), or
—C(═O)—(CH₂)ₘ—S(v₃);
   where v₃ and v₄ are each independently H, a C₁ to C₁₇ linear or branched alkyl chain or a C₂ to C₁₉ linear or branched alkyl acyl chain, on the proviso that if one of v₃ or v₄ is an alkyl acyl chain, then the other of v₃ or v₄ is H, and m is 0 to 17.

In a related aspect, an amino acid surrogate of formula I is at the C-terminus position of the mimetic, and at least one of R and R′ is a natural or unnatural amino acid side chain moiety or derivative of an amino acid side chain moiety with a heteroatom group comprising at least one nitrogen atom, and the remaining one of R and R′ is H or a natural or unnatural amino acid side chain moiety or derivative of an amino acid side chain moiety.

In a related embodiment, the invention provides a mimetic which binds to NPR, including but not limited to a receptor for human ANP, BNP or CNP, wherein such mimetic includes a plurality of amino acid residues and at least one amino acid surrogate located at any position other than the C-terminus position or N-terminus position and covalently bonded by two peptide bonds, and of formula II:

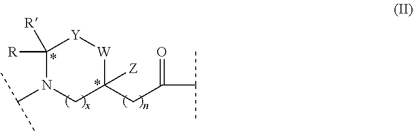

where R and R′ are each independently H or a natural or unnatural amino acid side chain moiety or derivative of an amino acid side chain moiety; x is 1 or 2; Y is CH₂ or C═O; W is CH₂, NH or NR′″; Z is H or CH₃; R′ is an acyl, a C₁ to C₁₇ linear or branched alkyl chain, a C₂ to C₁₉ linear or branched alkyl acyl chain, a C₁ to C₁₇ linear or branched omega amino aliphatic, or a C₁ to C₁₇ linear or branched omega amino aliphatic acyl; n is 0, 1 or 2; the carbon atoms marked with an asterisk can have any stereochemical configuration; and the broken lines indicate the bond forming a peptide bond.

Where the surrogate of formula I is at the C-terminus of the mimetic, it is covalently bonded thereto by a single peptide bond, such that the surrogate has the formula:

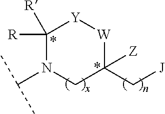

where the broken line indicates the bond forming a peptide bond. Where the surrogate is at the N-terminus of the mimetic it is preferably of formula I, and is covalently bonded thereto by a single bond peptide bond, such that the surrogate has the formula:

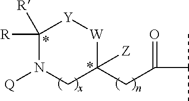

where the broken line indicates the bond forming a peptide bond. However, where the surrogate is at other than at the N-terminus or C-terminus of the mimetic, it is preferably of formula II and is covalently bonded thereto by two peptide bonds.

In another aspect, the invention provides a method of prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, by administration of a pharmaceutically effective amount of a mimetic which binds to NPRA, wherein such mimetic is of formula IV:

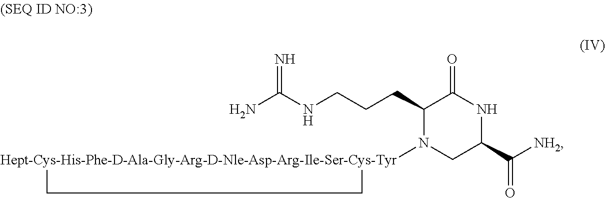

or a pharmaceutically acceptable salt of the mimetic of formula IV.

In different embodiments of the invention, one amino acid surrogate may be employed in a mimetic of the invention, two amino acid surrogates may be employed in a mimetic of the invention, or more than two amino acid surrogates may be employed in a mimetic of the invention.

A primary object of the present invention is to provide methods for the treatment of patients with a defect, condition, syndrome, disease or mutation resulting in less than optimal or desirable levels of active ANP₉₉₋₁₂₆.

Another object of the present invention is to provide methods for the prophylaxis or treatment of who have or are at risk of having less than optimal or desirable levels of active ANP₉₉₋₁₂₆.

Another object of the present invention is to provide compositions and methods for the treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, by administration of a natriuretic peptide mimetic wherein the mimetic exhibits, upon administration to a mammal, one or more advantages relative to the corresponding amino acid sequence not comprising an amino acid surrogate, the advantages selected from the group consisting of increased resistence to enzymatic degradation, increased circulation half life or mean residence time, increased bioavailability, increased efficacy, prolonged duration of effect and combinations of the foregoing.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has at least 10% of the maximal cGMP stimulating activity as the same concentration of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has at least 50% of the maximal cGMP stimulating activity as the same concentration of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has at least 100% of the maximal cGMP stimulating activity as the same concentration of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has more than 100% of the maximal cGMP stimulating activity as the same concentration of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has an equilibrium receptor binding affinity, determined by the Ki (nM) value, no greater than two log orders higher than the Ki (nM) value of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has an equilibrium receptor binding affinity, determined by the Ki (nM) value, no greater than three times higher than the Ki (nM) value of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has an equilibrium receptor binding affinity, determined by the Ki (nM) value, equal to or less than than the Ki (nM) value of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has an equilibrium receptor binding affinity, determined by the Ki (nM) value, less than the Ki (nM) value of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the mimetic has a receptor binding affinity with respect to a natriuretic peptide receptor greater than the receptor binding affinity of the corresponding amino acid sequence not comprising an amino acid surrogate.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the corresponding amino acid sequence not comprising an amino acid surrogate has at least about 60% homology with the sequence H-Met-cyclo(Cys-His-Phe-Gly-Gly-Arg-Met-Asp-Arg-Ile-Ser-Cys)-Tyr-Arg-NH₂ (SEQ ID NO:1).

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the corresponding amino acid sequence not comprising an amino acid surrogate has at least about 80% homology with the sequence H-Met-cyclo(Cys-His-Phe-Gly-Gly-Arg-Met-Asp-Arg-Ile-Ser-Cys)-Tyr-Arg-NH₂ (SEQ ID NO:1).

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the corresponding amino acid sequence not comprising an amino acid surrogate has at least about 60% homology with the sequence H-Met-cyclo(Xaa-His-Phe-Gly-Gly-Arg-Met-Asp-Arg-Ile-Ser-Xaa)-Tyr-Arg-NH₂ (SEQ ID NO:2), where Xaa are each independently any amino acid residue which together form a cyclic peptide.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a natriuretic peptide mimetic wherein the corresponding amino acid sequence not comprising an amino acid surrogate has at least about 80% homology with the sequence H-Met-cyclo(Xaa-His-Phe-Gly-Gly-Arg-Met-Asp-Arg-Ile-Ser-Xaa)-Tyr-Arg-NH₂ (SEQ ID NO:2), where Xaa are each independently any amino acid residue which together form a cyclic peptide.

Another object of the present invention is to provide natriuretic peptide mimetics which increase NPRA agonism and which may be administered to patients with a defect, condition, syndrome, disease or mutation resulting in a functional active ANP₉₉₋₁₂₆ deficiency, including patients who are diagnosed with cardiovascular disease, including but not limited to hypertension, acute coronary syndrome, cardiomyopathy, cardiac remodeling, left-ventricular hypertrophy, congestive heart failure, heart failure, high blood pressure and coronary artery disease.

Another object of the present invention is to provide natriuretic peptide mimetics which may be administered to patients with a defect, condition, syndrome, disease or mutation resulting in a functional active ANP₉₉₋₁₂₆ deficiency for prophylaxis or prevention, or prevention of worsening, of cardiovascular disease, including but not limited to hypertension, acute coronary syndrome, cardiomyopathy, cardiac remodeling, left-ventricular hypertrophy, congestive heart failure, heart failure, high blood pressure and coronary artery disease.

Another object of the present invention is to provide natriuretic peptide mimetics and formulations for the same which may be administered by subcutaneous or intravenous injection.

Another object of the present invention is to provide natriuretic peptide mimetics and formulations for the same which may be administered by infusion, including subcutaneous infusion.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, by administration of a natriuretic peptide mimetic which increases NPRA agonism, and preferably a natriuretic peptide mimetic with increased resistance to degradation or clearance.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing natriuretic peptide mimetics which have either a circulation half-life or mean residence time at least ten times, and preferably at least twenty times, that of endogenous active ANP₉₉₋₁₂₆.

Another object of the present invention is to provide methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing natriuretic peptide mimetics with reduced clearance through NPRC, compared to either ANP or BNP, and which preferably bind to NPRC with decreased affinity compared to binding to NPRA.

Other objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate one or more embodiments of the present invention and, together with the description, serves to explain the principles of the invention. The drawings are only for the purpose of illustrating one or more preferred embodiments of the invention and are not to be construed as limiting the invention.

FIG. 1 is a plot of amounts of the natriuretic peptide mimetic of formula IV, hANP and hCNP remaining intact after incubation in a 50 μM solution of hNEP.

FIG. 2 is a plot of plasma aldosterone (pg/mL) at selected days in rats with renovascular hypertension and heart failure utilizing a “2 kidney, 1 clip” (2K1C) model, comparing control animals receiving saline injections to experimental animals receiving 0.03 mg/kg per day of the natriuretic peptide mimetic of formula IV.

FIG. 3 is a plot of the heart weight to body weight ratio at six weeks post treatment in sham-treated animals receiving either saline or 0.03 mg/kg of the natriuretic peptide mimetic of formula IV and 2K1C-induced animals receiving either saline or 0.03 mg/kg of the mimetic of formula IV.

FIG. 4 is a plot of the heart weight to body weight ratio (mg/g) at two weeks post thoracic aortic banding in conditional cardiomyocyte restricted corin knockout mice and wild-type controls, receiving either phosphate-buffered saline (control) or 0.01 mg/kg/min of the natriuretic peptide mimetic of formula IV for two weeks via an implanted osmotic infusion pump.

### DETAILED DESCRIPTION OF THE INVENTION

Natriuretic peptide mimetics made of a plurality of amino acid residues, at least one ring-constrained amino acid surrogate and optionally at least one prosthetic group, are described generally in U.S. patent application Ser. No. 11/694,260 entitled “Cyclic Natriuretic Peptide Constructs”, filed on Mar. 30, 2007, now U.S. Pat. No. 7,622,440, issued on Nov. 24, 2009; U.S. patent application Ser. No. 11/694,358 entitled “Linear Natriuretic Peptide Constructs”, filed on Mar. 30, 2007, now U.S. Pat. No. 7,795,221, issued on Sep. 14, 2010; U.S. patent application Ser. No. 11/694,181, entitled “Amino Acid Surrogates for Peptidic Constructs”, filed on Mar. 30, 2007, now U.S. Pat. No. 7,964,1811, issued Jun. 21, 2011; U.S. Ser. No. 12/572,284 entitled “Amide Linkage Natriuretic Peptide Constructs”, filed on Oct. 2, 2009, now U.S. Pat. No. 8,580,746, issued Nov. 12, 2013; U.S. patent application Ser. No. 12/622,055 entitled “Cyclic Natriuretic Peptide Constructs”, filed on Nov. 19, 2009, now U.S. Pat. No. 8,580,747, issued on Nov. 12, 2013; and U.S. patent application Ser. No. 13/653,508 entitled “Uses of Natriuretic Peptide Constructs”, filed on Oct. 17, 2012. The methods, formulations and uses of this invention may be practiced with a natriuretic peptide mimetic as disclosed in any one of the foregoing patent applications, and accordingly the specification and claims of each of the foregoing patents and patent applications are incorporated herein by reference as if set forth in full.

The methods, formulations and uses of this invention may alternatively be practiced with any natriuretic peptide mimetic that binds to and activates a natriuretic peptide receptor, including but not limited to those disclosed in U.S. Patent Application Publication Nos. 2004/0002458; 2004/0063630; 2004/0077537; 2005/0113286; 2005/0176641; 2006/0030004; 2013/0303454; 2013/0324472; 2013/0345136; 2013/0345349; 2014/0005358; and 2014/0066367; International Patent Application Nos. WO 85/04870; WO 85/04872; WO 88/03537; WO 88/06596; WO 89/10935; WO 89/05654; WO 90/01940; WO 90/14362; WO 92/06998; WO 95/13296; WO 99/08510; WO 99/12576; WO 01/016295; WO 2004/047871; WO 2005/072055; WO 2009/149161; WO 2011/075471; WO 2013/151766; WO 2013/151767; and WO 2013/090931; and U.S. Pat. Nos. 4,496,544; 4,609,725; 4,656,158; 4,673,732; 4,716,147; 4,757,048; 4,764,504; 4,804,650; 4,816,443; 4,824,937; 4,861,755; 4,904,763; 4,935,492; 4,952,561; 5,047,397; 5,057,495; 5,057,603; 5,091,366; 5,095,004; 5,106,834; 5,114,923; 5,159,061; 5,204,328; 5,212,286; 5,352,587; 5,376,635; 5,418,219; 5,665,704; 5,846,932; 5,583,108; 5,965,533; 6,028,055; 6,083,982; 6,124,430; 6,150,402; 6,407,211; 6,525,022; 6,586,396 6,818,619; 8,546,523; 8,551,938; and 8,598,121. Accordingly the specification and claims of each of the foregoing patents and patent applications are incorporated herein by reference as if set forth in full.

The invention provides methods and uses of natriuretic peptide mimetics for the prophylaxis or treatment of human disease, including cardiovascular disease or renal disease, in patients with a defect, condition, syndrome, disease or mutation resulting in a functional active natriuretic peptide deficiency, by administration of a pharmaceutically effective amount of a mimetic as described herein. The mimetic is in one aspect an agonist binding to and activating NPRA or NPRB.

The invention further provides methods and uses of natriuretic peptide mimetics for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, by administration of a pharmaceutically effective amount of a mimetic as described herein.

The natriuretic peptide mimetics may comprise one or more ring-constrained amino acid surrogates as defined herein, which surrogates may further comprise a conventional amino protected N-terminus, using a protecting group such as Fmoc, and a reactive carboxyl C-terminus, such that they may thus be employed in conventional peptide synthesis methodologies, it being understood that if the amino acid surrogate is at the C-terminus position of the mimetic, that other than a carboxyl terminus may be employed on such surrogate.

In a related preferred embodiment, the mimetic further includes at least one prosthetic group. Preferred prosthetic groups include polymeric groups comprising repeat units including one or more carbon and hydrogen atoms, and optionally other atoms, including oxygen. Such polymeric groups are preferably water-soluble polymers, and are preferably poly(alkylene oxide), poly(vinyl pyrrolidone), poly(vinyl alcohol), polyoxazoline or poly(acryloylmorpholine). A preferred poly(alkylene oxide) is poly(ethylene glycol) (PEG), optionally derivatized with a linking group.

In one particularly preferred embodiment, the invention employs a mimetic, comprising an amino acid sequence which binds to a natriuretic peptide receptor, preferably NPRA, wherein one or more amino acid residues in such amino acid sequence which binds to a natriuretic peptide receptor is substituted with an amino acid surrogate of formula I. In one aspect, the amino acid sequence which binds to a natriuretic peptide receptor, and preferably NPRA, is, prior to substitution, H-Met-cyclo(Cys-His-Phe-Gly-Gly-Arg-Met-Asp-Arg-Ile-Ser-Cys)-Tyr-Arg-NH₂ (SEQ ID NO:1).

In yet another embodiment the invention employs a mimetic that binds to a receptor for a natriuretic peptide, including a receptor for ANP, and includes at least one amino acid surrogate of formula I or II.

In one embodiment, the invention employs a cyclic mimetic of formula III:

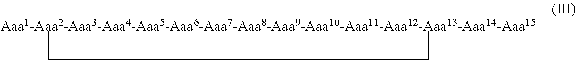

where

Aaa¹ is an L- or D-isomer of an α-amino acid or β-amino acid or an α,α-disubstituted amino acid derived from an α-amino acid, including where Aaa¹ is an L- or D-isomer of an α-amino acid or β-amino acid including or derived from Nle, Ala, Leu, Ile, Val, Arg, Phe, Lys, Tyr, Asp, Nva, Met, Met(O), or Met(O₂), or an α,α-disubstituted amino acid derived from Nle, Ala, Leu, Ile, Val, Arg, Phe, Lys, Tyr, Asp, Nva, Met, Met(O), or Met(O₂), including all (R) or (S) configurations of α,α-disubstituted amino acids where the substituents are different, or Aaa¹ is an acyl comprising a C₂ to C₁₈ linear alkyl, a C₃ to C₁₇ branched alkyl, a C₂ to C₁₈ linear alkenyl or alkynyl or a C₃ to C₁₈ branched alkenyl or alkynyl, or Aaa¹ is an amino acid surrogate of the structure:

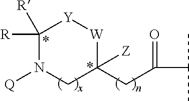

wherein the broken line indicates a peptide bond; R and R′ are independently H, a linear or branched C₁ to C₆ aliphatic chain, —(CH₂)_{y}—S—CH₃, —(CH₂)_{y}—S(═O)—CH₃, —(CH₂)_{y}—S(O₂)—CH₃, a bond and a cyclopropane, cyclobutane, cyclopentane, or cyclohexane ring, or a C₁ to C₃ aliphatic chain and a cyclopropane, cyclobutane, cyclopentane, or cyclohexane ring; x is 1 or 2; Y is CH₂ or C═O; W is CH₂, NH or NR′″; Z is H or CH₃; Q is —H, —(CH₂)ₘ—N(v₃)(v₄), —(CH₂)ₘ—CH₃, —(CH₂)ₘ—O(v₃), —(CH₂)ₘ—C(═O)-(v₃), —(CH₂)ₘ—C(═O)—O-(v₃), —(CH₂)ₘ—S(v₃), —C(═O)—(CH₂)ₘ—CH₃, —C(═O)—(CH₂)ₘ—N(v₃)(v₄), —C(═O)—(CH₂)ₘ—C(═O)-(v₃), —C(═O)—(CH₂)ₘ—O(v₃), or —C(═O)—(CH₂)ₘ—S(v₃); R′″ is an acyl, a C₁ to C₁₇ linear or branched alkyl chain, a C₂ to C₁₉ linear or branched alkyl acyl chain, a C₁ to C₁₇ linear or branched omega amino aliphatic, or a C₁ to C₁₇ linear or branched omega amino aliphatic acyl; n is 0, 1 or 2; m is 0 to 17; y is 1 to 5; v₃ and v₄ are each independently H, a C₁ to C₁₇ linear or branched alkyl chain or a C₂ to C₁₉ linear or branched alkyl acyl chain, on the proviso that if one of v₃ or v₄ is an alkyl acyl chain, then the other of v₃ or v₄ is H; and the carbon atoms marked with an asterisk can have any stereochemical configuration;

Aaa² and Aaa¹³ are the same or different, and are each L- or D-isomer amino acid residues forming a cyclic bridge through the side chains of each of Aaa² and Aaa¹³, wherein the linking group of the cyclic bridge is —S—S—, —S—CH₂—S—, —S—CH₂—, —CH₂—S—, —C(═O)—NH—, —NH—C(═O)—, —CH₂—NH—, —NH—CH₂—, —CH₂—S(O)ₙ— where n is 1 or 2, —S(O)ₙ—CH₂— where n is 1 or 2, —CH₂—CH₂—, —CH═CH— (E or Z), —C≡C—, —C(═O)—O—, —O—C(═O)—, —C(═O)—CH₂—, —CH₂—C(═O)—, —O—C(═O)—NH—, —NH—C(═O)—O—, or —NH—C(═O)—NH—;

Aaa³ is an L- or D-isomer of an α-amino acid or β-amino acid including or derived from His, Ala, Ser, Thr, Lys, HLys, Orn, Cys, HCys, Dap, or Dab, or an α,α-disubstituted amino acid derived from His, Ala, Ser, Thr, Lys, HLys, Orn, Cys, HCys, Dap, or Dab, including all (R) or (S) configurations of α,α-disubstituted amino acids where the substituents are different, or Aaa³ is an amino acid surrogate of the structure:

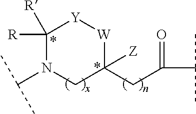

where R and R′ are independently H or an amino acid side chain moiety of His, Ala, Ser, Thr, Lys, HLys, Orn, Cys, HCys, Dap, or Dab or a derivative of an amino acid side chain moiety of His, Ala, Ser, Thr, Lys, HLys, Orn, Cys, HCys, Dap, or Dab; x is 1 or 2; Y is CH₂ or C═O; W is CH₂, NH or NR′″; Z is H or CH₃; R′ is an acyl, a C₁ to C₁₇ linear or branched alkyl chain, a C₂ to C₁₉ linear or branched alkyl acyl chain, a C₁ to C₁₇ linear or branched omega amino aliphatic, or a C₁ to C₁₇ linear or branched omega amino aliphatic acyl; and n is 0, 1 or 2;

Aaa⁴ is an L- or D-isomer of an α-amino acid or β-amino acid including or derived from substituted or unsubstituted Phe, HPhe or Pgl, or Tyr, Leu, Ile, Val, Ala, Nle, Nva or Tle, or an α,α-disubstituted amino acid derived from substituted or unsubstituted Phe, HPhe or Pgl, or Tyr, Leu, Ile, Val, Ala, Nle, Nva or Tle, including all (R) or (S) configurations of α,α-disubstituted amino acids where the substituents are different, or Aaa⁴ is an amino acid surrogate as for Aaa³ where R and R′ are independently H or an amino acid side chain moiety of substituted or unsubstituted Phe, HPhe or Pgl, or Tyr, Leu, Ile, Val, Ala, Nle, Nva or Tle or a derivative of an amino acid side chain moiety of substituted or unsubstituted Phe, HPhe or Pgl, or Tyr, Leu, Ile, Val, Ala, Nle, Nva or Tle;

Aaa⁵ is Gly, Sar, an L- or D-isomer of an α-amino acid or β-amino acid including or derived from Ala, or Aib, which is the α,α-disubstituted amino acid derived from Ala, or Aaa⁵ is an amino acid surrogate as for Aaa³ where R and R′ are independently H or —CH₃;

Aaa⁶ is Gly, Sar, an L- or D-isomer of an α-amino acid or β-amino acid including or derived from Ala, or Aib, or Aaa⁶ is an amino acid surrogate as for Aaa³ where R and R′ are independently H or —CH₃;

Aaa⁷ is an L- or D-isomer of an α-amino acid or β-amino acid including or derived from Arg, His, Ala, Ser, HSer, Thr, Lys, HLys, Orn, Cys, HCys, Cit, Abu, Dap, or Dab, or an α,α-disubstituted amino acid derived from Arg, His, Ala, Ser, HSer, Thr, Lys, HLys, Orn, Cys, HCys, Cit, Abu, Dap, or Dab, including all (R) or (S) configurations of α,α-disubstituted amino acids where the substituents are different, or Aaa⁷ is an amino acid surrogate as for Aaa³ where R and R′ are independently H or an amino acid side chain moiety of Arg, His, Ala, Ser, HSer, Thr, Lys, HLys, Orn, Cys, HCys, Abu, Dap, or Dab or a derivative of an amino acid side chain moiety of Arg, His, Ala, Ser, HSer, Thr, Lys, HLys, Orn, Cys, HCys, Abu, Dap, or Dab;

Aaa⁸ is Gly, an L- or D-isomer of an α-amino acid or β-amino acid including or derived from Nle, Ile, Leu, Val, Phe, Ala, Nva, Met(O), Met(O₂), or Tle, or an α,α-disubstituted amino acid derived from Nle, Ile, Leu, Val, Phe, Ala, Nva, Met(O), Met(O₂), or Tle, including all (R) or (S) configurations of α,α-disubstituted amino acids where the substituents are different, or Aaa⁸ is an amino acid surrogate as for Aaa³ where R and R′ are independently H or an amino acid side chain moiety of Nle, Ile, Leu, Val, Phe, Ala, Nva, Met(O), Met(O₂), or Tle, or a derivative of an amino acid side chain moiety of Nle, Ile, Leu, Val, Phe, Ala, Nva, Met(O), Met(O₂), or Tle;

Aaa⁹ is an L- or D-isomer of an α-amino acid or β-amino acid including or derived from Asp, Glu, His, Ala, Ser, Thr, Lys, HLys, Cys, HCys, Met(O), Met(O₂), Orn, Dap, or Dab, or an α,α-disubstituted amino acid derived from Asp, Glu, His, Ala, Ser, Thr, Lys, HLys, Cys, HCys, Met(O), Met(O₂), Orn, Dap, or Dab, including all (R) or (S) configurations of α,α-disubstituted amino acids where the substituents are different, or Aaa⁹ is an amino acid surrogate as for Aaa³ where R and R′ are independently H or an amino acid side chain moiety of Asp, Glu, His, Ala, Ser, Thr, Lys, HLys, Cys, HCys, Met(O), Met(O₂), Orn, Dap, or Dab or a derivative of an amino acid side chain moiety of Asp, Glu, His, Ala, Ser, Thr, Lys, HLys, Cys, HCys, Met(O), Met(O₂), Orn, Dap, or Dab;

Aaa¹⁰ is an L- or D-isomer of an α-amino acid or β-amino acid including or derived from Arg, His, Ala, Ser, Thr, Lys, HLys, Cys, HCys, Cit, Met(O), Orn, Dap, or Dab, or an α,α-disubstituted amino acid derived from Arg, His, Ala, Ser, Thr, Lys, HLys, Cys, HCys, Cit, Met(O), Orn, Dap, or Dab, including all (R) or (S) configurations of α,α-disubstituted amino acids where the substituents are different, or Aaa¹⁰ is an amino acid surrogate as for Aaa³ where R and R′ are independently H or an amino acid side chain moiety of Arg, His, Ala, Ser, Thr, Lys, HLys, Cys, HCys, Met(O), Orn, Dap, or Dab or a derivative of an amino acid side chain moiety of Arg, His, Ala, Ser, Thr, Lys, HLys, Cys, HCys, Met(O), Orn, Dap, or Dab;

Aaa¹¹ is Gly or a D- or L-isomer of an α-amino acid or β-amino acid including or derived from Nle, Ile, Leu, Val, Phe, Ala, Nva, Cys, HCys, Abu or Tle, or an α,α-disubstituted amino acid derived from Nle, Ile, Leu, Val, Phe, Ala, Nva, Cys, HCys, Abu or Tle, including all (R) or (S) configurations of α,α-disubstituted amino acids where the substituents are different, or Aaa¹¹ is an amino acid surrogate as for Aaa³ where R and R′ are independently H or an amino acid side chain moiety of Nle, Ile, Leu, Val, Phe, Ala, Nva, Cys, HCys, Abu or Tle or a derivative of an amino acid side chain moiety of Nle, Ile, Leu, Val, Phe, Ala, Nva, Cys, HCys, Abu or Tle;

Aaa¹² is Gly, an L- or D-isomer of an α-amino acid or β-amino acid including or derived from Ser, Nle, Ile, Leu, Val, Phe, Ala, Nva, Arg, Lys, Orn, Cys, HCys, Abu or Tle, or an α,α-disubstituted amino acid derived from Ser, Nle, Ile, Leu, Val, Phe, Ala, Nva, Arg, Lys, Orn, Cys, HCys, Abu or Tle, including all (R) or (S) configurations of α,α-disubstituted amino acids where the substituents are different, or Aaa¹² is an amino acid surrogate as for Aaa³ where R and R′ are independently H or an amino acid side chain moiety of Ser, Nle, Ile, Leu, Val, Phe, Ala, Nva, Arg, Lys, Orn, Cys, HCys, Abu or Tle or a derivative of an amino acid side chain moiety of Ser, Nle, Ile, Leu, Val, Phe, Ala, Nva, Arg, Lys, Orn, Cys, HCys, Abu or Tle;

Aaa¹⁴ is an L- or D-isomer of an α-amino acid or β-amino acid including or derived from substituted or unsubstituted Phe, HPhe or Pgl, or Tyr, Leu, Ile, Val, Ala, Lys, Orn, Nle, Nva or Tle, or an α, α-disubstituted amino acid derived from substituted or unsubstituted Phe, HPhe or Pgl, or Tyr, Leu, Ile, Val, Ala, Lys, Orn, Nle, Nva or Tle, including all (R) or (S) configurations of α,α-disubstituted amino acids where the substituents are different, or Aaa¹⁴ is an amino acid surrogate of the structure of formula II as for Aaa³ where R and R′ are independently H or an amino acid side chain moiety of substituted or unsubstituted Phe, HPhe or Pgl, or Tyr, Leu, Ile, Val, Ala, Lys, Orn, Nle, Nva or Tle or a derivative of an amino acid side chain moiety of substituted or unsubstituted Phe, HPhe or Pgl, or Tyr, Leu, Ile, Val, Ala, Lys, Orn, Nle, Nva or Tle; and

Aaa¹⁵ is a D- or L-isomer of an α-amino acid or β-amino acid including or derived from Ala, Arg, Orn, Lys, Ala, Dap, Dab, HArg, or HLys, or an α,α-disubstituted amino acid derived from Ala, Arg, Orn, Lys, Ala, Dap, Dab, HArg, or HLys, including all (R) or (S) configurations of α,α-disubstituted amino acids where the substituents are different, or Aaa¹⁵ is an amino acid surrogate of the structure:

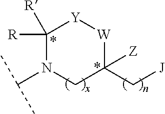

wherein the broken line indicates a peptide bond; at least one of R and R′ is —(CH₂)_{y}—R″ and if one, the remaining of R and R′ is H, where R″ is:
—NH₂,
—NH—C(═NH)—NH₂,
—NH—(CH₂)_{y}—NH₂,
—NH—C(═O)—NH₂,
—C(═O)—NH₂,
—C(═O)—NH—CH₃,
—C(═O)—NH—(CH₂)_{y}—NH₂,
—NH—C(═NH)—NH-Me,
—NH—C(═NH)—NH-Et,
—NH—C(═NH)—NH—Pr,
—NH—C(═NH)—NH—Pr-i,
—NH—C(═O)—CH₃,
—NH—C(═O)—CH₂—CH₃,
—NH—C(═O)—CH—(CH₃)₂,
—NH—C(═O)—O—CH₃,
—NH—C(═O)—O—CH₂—CH₃,
—NH—C(═O)—O—C—(CH₃)₃,
—NH—C(═O)—NH—CH₃,
—NH—C(═N—C(═O)—O—C—(CH₃)₃)—NH—C(═O)—O—C—(CH₃)₃,
—N(C(═O)—O—C—(CH₃)₃)—C(═NH)—NH—C(═O)—O—C—(CH₃)₃,

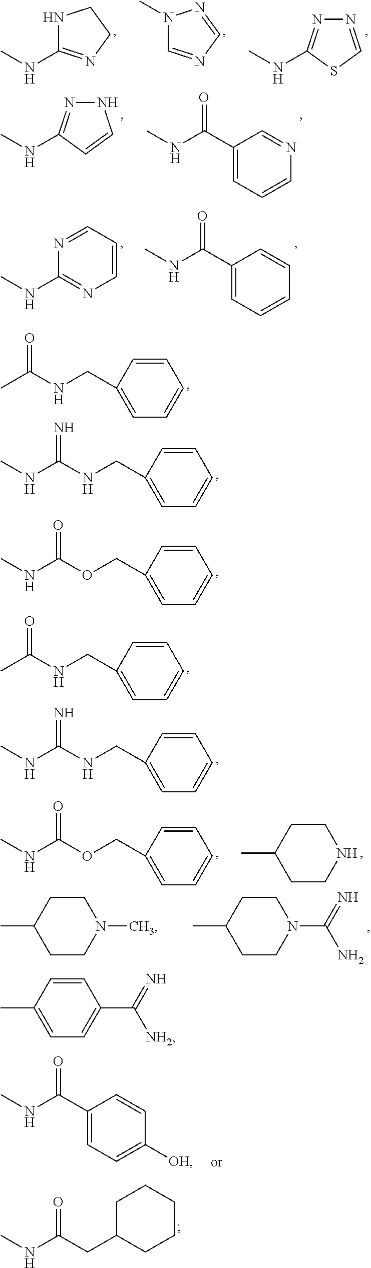

x is 1 or 2;
Y is CH₂ or C═O;
W is CH₂, NH or NR′″;
Z is H or CH₃;
J is
   —H,
   —(CH₂)ₘ—OH, —C(═O)—CH₂)ₘ—OH,
   —C(═O)—CH₂)ₘ—N((v₁)(v₂),
   —C(═O)—O—(CH₂)ₘ—CH₃,
   —O—(CH₂)ₘ—CH₃, —O—(CH₂)ₘ—N(v₁)(v₂),
   —O—(CH₂)ₘ—OH, —C(═O)—NH—(CH₂)ₘ—CH₃,
   —C(═O)—NH—(CH₂)ₘ—N(v₁)(v₂),
   —C(═O)—NH—(CH₂)ₘ—S(v₁),
   —C(═O)—N—((CH₂)ₘ—N(v₁)(v₂))₂,
   —C(═O)—NH—CH(—C(═O)—OH)—(CH₂)ₘ—N(v₁)(v₂),
   —C(═O)—NH—(CH₂)ₘ—NH—C(═O)—CH(N(v₁)(v₂))((CH₂)ₘ—N(v₁)(v₂)),
   —C(═O)—NH—CH(—C(═O)—N(v₁)(v₂))—(CH₂)ₘ—N(v₁)(v₂),
   an omega amino aliphatic, terminal aryl or aralkyl group,
   any single natural or unnatural α-amino acid, β-amino acid or α,α-disubstituted amino acid in combination with one of the foregoing groups defining J,
   or any single natural or unnatural α-amino acid, β-amino acid or α,α-disubstituted amino acid, including all (R) and (S) configurations of any of the foregoing;
R′″ is an acyl, a C₁ to C₁₇ linear or branched alkyl chain, a C₂ to C₁₉ linear or branched alkyl acyl chain, a C₁ to C₁₇ linear or branched omega amino aliphatic, or a C₁ to C₁₇ linear or branched omega amino aliphatic acyl;
v₁ and v₂ are each independently H or a C₁ to C₁₇ linear or branched alkyl chain;
n is 0, 1 or 2;
m is 0 to 17;
y is 1 to 5; and
the carbon atoms marked with an asterisk can have any stereochemical configuration;

on the proviso that at least one of Aaa¹, Aaa³ through Aaa¹², Aaa¹⁴ or Aaa¹⁵ is an amino acid surrogate.

A related embodiment of formula Ill provides a mimetic where one or more of Aaa¹, Aaa³ to Aaa¹², Aaa¹⁴ or Aaa¹⁵ is an amino acid surrogate as defined above, and where a prosthetic group, as hereafter defined, is attached to a reactive group of a side chain of an amino acid residue at one or more of Aaa¹, Aaa³ to Aaa¹², Aaa¹⁴ or Aaa¹⁵, to a reactive R or R′ group of an amino acid surrogate at Aaa³ to Aaa¹² or Aaa¹⁴, directly or through a Q group to the terminal amine of an amino acid surrogate at Aaa¹, to a reactive terminal carboxyl of an amino acid surrogate at Aaa¹⁵, or to a reactive group forming a part of J of an amino acid surrogate at Aaa¹⁵. The reactive group to which the one or more prosthetic groups are covalently bonded may be a primary amine, a secondary amine, a carboxyl group, a thiol group or a hydroxyl group. In one aspect, the prosthetic group may be covalently bound to a reactive amine in position Aaa¹, Aaa³, Aaa⁷, Aaa¹⁰, Aaa¹², or Aaa¹⁵, or a combination of the foregoing. In another aspect, the prosthetic group may be covalently bound to a reactive carboxyl in position Aaa⁹ or Aaa¹⁵, or both. In another aspect, the prosthetic group may be covalently bound to a reactive thiol in position Aaa³, Aaa⁷, Aaa⁹, Aaa¹⁰, Aaa¹¹, or Aaa¹², or a combination of the foregoing.

In a preferred aspect of the mimetic of formula III, one, two or three of Aaa¹ to Aaa¹⁵ (excluding Aaa² and Aaa¹³) are an amino acid surrogate of one of the foregoing formulas. In a first particularly preferred aspect, one of Aaa¹, Aaa⁵ and Aaa¹⁵ is an amino acid surrogate. In a second particularly preferred aspect, two of Aaa¹, Aaa⁵ and Aaa¹⁵ are amino acid surrogates. In a third particularly preferred aspect, each of Aaa¹, Aaa⁵ and Aaa¹⁵ are amino acid surrogates. In another particularly preferred aspect, one, two or three of Aaa¹, Aaa⁵ and Aaa¹⁵ are amino acid surrogates, and the mimetic is a cyclic mimetic formed by disulfide bond formation through the side chains of Aaa² and Aaa¹³. In another particularly preferred aspect, where two or more of Aaa¹ to Aaa¹⁵ are amino acid surrogates the amino acid surrogates are not contiguous, which is to say that each amino acid surrogate is separate from each other amino acid surrogate by at least one amino acid residue being interposed therebetween in the primary sequence.

In yet another preferred embodiment, in the mimetic of formula III at least one of Aaa³, Aaa⁵, Aaa⁶, Aaa⁷, Aaa⁹, Aaa¹⁰, or Aaa¹² is an L- or D-isomer of Ala, preferably an L-isomer of Ala.

In a particularly preferred embodiment, the mimetic of formula III is a natriuretic peptide mimetic of formula IV:

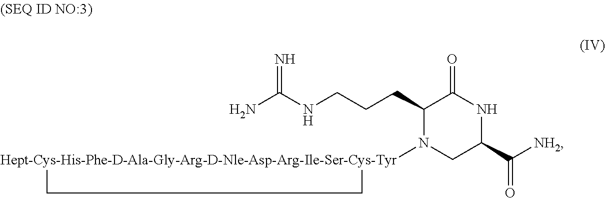

or a pharmaceutically acceptable salt of the mimetic of formula IV.

In yet another embodiment, the invention provides methods for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency, utilizing a mimetic of formula III further comprising one or more non-peptide bonds. Non-peptide bonds may be employed to decrease the susceptibility of a mimetic of the invention to degradation, such as improving the in vivo stability of mimetics towards tryptic-like proteases by replacing the native peptide bond before each Lys or Arg residue with a non-peptide bond, such as an isostere of an amide, a substituted amide or a peptidomimetic linkage. In one specific embodiment, native peptide bonds are replaced with peptide bonds having a reversed polarity. In general, any non-peptide bond may be employed, and may be utilized between any two residues. A non-peptide bond includes bonds in which the carbon atom participating in the bond between two residues is reduced from a carbonyl carbon to a methylene carbon, such as a non-peptide bond —CH₂—NH— or its isostere —NH—CH₂—, or the use of other bonds such as —CH₂—S—, —CH₂—O—, or —C(═O)—CH₂— or an isostere of any of the foregoing, or —CH₂—CH₂— or —CH═CH—. In general, non-peptide bonds include an imino, ester, hydrazine, semicarbazide, oxime, or azo bond.

The natriuretic peptide mimetics defined above may include one or more prosthetic groups. Prosthetic groups may be employed to modulate the residence time in circulation, to modulate bioavailability, modulate immunogenicity of mimetics, or the like. In general, prosthetic groups “modulate” by increasing the residence time, bioavailability or the like, as the case may be, but prosthetic groups may optionally decrease residence time, bioavailability or the like. A “prosthetic group” thus includes any compound conjugated, such as by a covalent bond, to a mimetic of any formula, for purposes of improving pharmacokinetic or pharmacodynamic properties of the mimetic. Preferred prosthetic groups include polymeric groups, comprising repeat units which in turn comprise one or more carbon and hydrogen atoms, and optionally other atoms, including oxygen atoms. Such polymeric groups are preferably water-soluble polymers, and are preferably poly(alkylene oxide), poly(vinyl pyrrolidone), poly(vinyl alcohol), polyoxazoline or poly(acryloylmorpholine). A preferred poly(alkylene oxide) is poly(ethylene glycol) (PEG). In addition to PEG, other poly(alkylene glycol) polymers may be employed, such as poly(propylene glycol) and poly(butylene glycol).

In one embodiment, the prosthetic group is one or more PEG polymers covalently bound to a reactive group of the mimetic. The PEG polymer, or other prosthetic group, may be covalently bound to a reactive group on the side chain of one or more amino acid residues, or may be covalently bound to a reactive group on an amino acid surrogate. Such reactive groups of an amino acid surrogate may include a group covalently bound, directly or through one or more intermediates, to Q or J, or may include a reactive group forming a part of R or R′.

If PEG is employed as the prosthetic group, the PEG polymer may have a molecular weight of from about 200 MW to about 50000 MW. The PEG polymer may be linear, and if linear, may be monofunctional, with a reactive group at one end and a non-reactive group at the other end, homobifunctional, with the same reactive group at each end, or heterobifunctional, with a different reactive group at each end. Alternatively, the PEG polymer may be branched, having generally a “Y”-shaped configuration, multi-armed, such as with two, three, four or eight arms, or other configurations known in the art. The PEG polymer preferably has at least one derivatized reactive group for linking to one or more defined groups on the mimetic of formula III, preferably by means of a covalent bond. The derivativized reactive group may link to, for example, an amine, hydroxyl, thiol, or carboxyl group on a mimetic, including on a terminal group of an amino acid residue, on a side chain of an amino acid residue, on a Q group of a surrogate, on a J group of a surrogate, or on an R or R′ group of a surrogate.

The PEG polymer preferably has, at one end, an end-cap group, such as a hydroxyl, alkoxy, substituted alkoxy, alkenoxy, substituted alkenoxy, alkynoxy, substituted alkynoxy, aryloxy or substituted aryloxy. The PEG polymer further preferably has, at at least one other end, a derivatized reactive group. In one embodiment, the PEG polymer is a linear or branched polyether with a terminal hydroxyl group, such as a monomethoxy PEG, which is derivatized with a linking group, such as an amine, maleimide or carboxylic acid. The available reactive groups of the mimetic dictate the derivatized linking group employed on the PEG polymer. Thus, in one embodiment, the N-terminal amine of the mimetic is employed, using a carboxylic acid derivatized PEG. In another embodiment, the C-terminal amine of the mimetic is employed, again using a carboxylic acid derivatized PEG. In yet another embodiment, if a Lys residue or homolog thereof is present in the mimetic, either the α or ε amino group thereof may be employed, again using a carboxylic acid derivatized PEG. Maleimide derivatized PEG may be employed with either a reactive thiol or hydroxyl group on the mimetic. Similarly, amine derivatized PEG may be employed with a reactive carboxyl group on any terminal group or side chain of an amino acid residue, on a Q group of a surrogate, on a J group of a surrogate, or on an R or R′ group of a surrogate.

Thus, in one aspect, PEG is activated with one or more electrophilic groups and may be employed for coupling to amino groups of the mimetic, including coupling to an ε amino group of a side chain or an N-terminal or C-terminal amine. Representative electrophilic reactive groups include succinimidyl α-methylbutanoate and other α-methylbutyric acid esters, as disclosed in U.S. Pat. Nos. 5,672,662 and 6,737,505, and may be be used with proteins, as disclosed in U.S. Patent Application Publication 2004/0235734. Alternatively, succinimidyl propionate may be employed as a reactive group, as disclosed in U.S. Pat. No. 5,567,662, or N-hydroxysuccinimide may be employed with a branched PEG, as disclosed in U.S. Pat. No. 5,932,462. The teachings of each of the foregoing patents and patent applications are incorporated by reference as if set forth in full.

In another aspect, PEG polymers are provided with one or more reactive aldehyde groups, and employed for coupling to a terminal primary amine, such as an N-terminal or C-terminal amine. In another aspect, PEG polymers are provided with one or more thiol-reactive groups, such as a maleimide, ortho-pyridyldisulfide, or thiol group, and are employed for coupling to a reactive thiol in the mimetic of formula III, such as a reactive thiol in a cysteine side chain or a reactive thiol in a Q group of a mimetic.

In one aspect, any of the methods, conjugates or schemes as disclosed in International Patent Publication Nos. WO 2010/033216; WO 2010/033207; WO 2004/047871, or any reference cited therein, may be employed with the natriuretic peptide mimetics employed in this invention. The teaching of the foregoing patent applications is incorporated by reference as if set forth in full.

In general, some form of chemical modification may be employed to make an active PEG derivative with a reactive group. The reactive group may be an active carbonate, an active ester, an aldehyde, or tresylate. In part, the reactive group of the PEG determines the amino acid terminal group or side chain moiety to which the PEG derivative is bound. In general, site specific PEGylation is preferred, in part because the resulting mimetic is homogeneous, minimizing loss of biological activity and reducing immunogenicity.

In one embodiment, the PEG has a molecular weight of from about 200 MW to about 50,000 MW, more preferably from about 2,000 MW to about 20,000 MW. In another embodiment, monomethoxy PEG, such as of the formula CH₃—O(CH₂—CH₂—O)ₙ—CH₂—CH₂—OH or CH₃—O(CH₂—CH₂—O)ₙ—H, where n is any integer from 2 to about 1200, is employed, preferably derivatized with an amine, maleimide or carboxylic acid linking group.

In another embodiment, the prosthetic group, such as PEG, is conjugated to a natriuretic peptide mimetic by means of an enzymatically labile linker as described in Veronese, F. M. and Pasut G.: “Pegylation, Successful approach to drug delivery.” Drug Discovery Today 10:1451-1458 (2005), and the methods disclosed therein are incorporated here by reference.

In another embodiment, the prosthetic group employed is a polymer with both a lipophilic moiety and a hydrophilic polymer moiety, as disclosed in U.S. Pat. Nos. 5,359,030 and 5,681,811. In a related embodiment, the prosthetic group employed is an oligomer conjugate with a hydrophilic component, such as a PEG polymer, and a lipophilic component, such as a branched fatty acid or alkyl chain, linked by a hydrolyzable bond, such as an ester bond, as disclosed in U.S. Pat. No. 6,309,633. In another related embodiment, the prosthetic group employed is an oligomer that includes poly(propylene glycol), and preferably at least two poly(propylene glycol) subunits, as disclosed in U.S. Pat. No. 6,858,580. The teachings of each of the foregoing patents and patent applications are incorporated by reference as if set forth in full.

In yet another embodiment, the teachings of U.S. Published Patent Application 2004/0203081 are incorporated here by reference, including specifically teachings relating to prosthetic groups, referred to in such application as “modifying moieties,” attached to various natriuretic compounds, and specifically oligomeric structures having a variety of lengths and configurations. In a related embodiment, the teachings of International Patent Publication WO 2004/047871 are incorporated by reference, including teachings related to “modifying moieties” attached by means of “modifying moiety conjugation sites” to natriuretic molecules binding to NPRA, it being understood that similar methods could be employed with natriuretic molecules binding to other natriuretic receptors.

Certain terms as used throughout the specification and claims are defined as follows.

The “mimetic” and “amino acid residue sequences” employed in this invention can be a) naturally-occurring, b) produced by chemical synthesis, c) produced by recombinant DNA technology, d) produced by biochemical or enzymatic fragmentation of larger molecules, e) produced by methods resulting from a combination of methods a through d listed above, or f) produced by any other means for producing peptides or amino acid sequences.

By employing chemical synthesis, a preferred means of production, it is possible to introduce various amino acids which do not naturally occur into the mimetic, modify the N- or C-terminus, and the like, thereby providing for improved stability and formulation, resistance to protease degradation, and the like, and to introduce one or more amino acid surrogates into the mimetic.

The term “peptide” as used throughout the specification and claims is intended to include any structure comprised of two or more amino acids, including chemical modifications and derivatives of amino acids. The amino acids forming all or a part of a peptide may be naturally occurring amino acids, stereoisomers and modifications of such amino acids, non-protein amino acids, post-translationally modified amino acids, enzymatically modified amino acids, and the like. The term “peptide” also includes dimers or multimers of peptides. A “manufactured” peptide includes a peptide produced by chemical synthesis, recombinant DNA technology, biochemical or enzymatic fragmentation of larger molecules, combinations of the foregoing or, in general, made by any other method.

The term “amino acid side chain moiety” used in this invention, including as used in the specification and claims, includes any side chain of any amino acid, as the term “amino acid” is defined herein. This thus includes the side chain moiety present in naturally occurring amino acids. It further includes side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs or structures designed to mimic amino acids, and the like. For example, the side chain moiety of any amino acid disclosed herein is included within the definition. A “derivative of an amino acid side chain moiety” as hereafter defined is included within the definition of an amino acid side chain moiety.

The “derivative of an amino acid side chain moiety” is a modification to or variation in any amino acid side chain moiety, including a modification to or variation in either a naturally occurring or unnatural amino acid side chain moiety, wherein the modification or variation includes: (a) adding one or more saturated or unsaturated carbon atoms to an existing alkyl, aryl, or aralkyl chain; (b) substituting a carbon in the side chain with another atom, preferably oxygen or nitrogen; (c) adding a terminal group to a carbon atom of the side chain, including methyl (—CH₃), methoxy (—OCH₃), nitro (—NO₂), hydroxyl (—OH), or cyano (—C≡N); (d) for side chain moieties including a hydroxy, thiol or amino groups, adding a suitable hydroxy, thiol or amino protecting group; or (e) for side chain moieties including a ring structure, adding one or ring substituents, including hydroxyl, halogen, alkyl, or aryl groups attached directly or through an ether linkage. For amino groups, suitable amino protecting groups include, but are not limited to, Z, Fmoc, Boc, Pbf, Pmc and the like.

The “amino acids” used in embodiments of the present invention, and the term as used in the specification and claims, include the known naturally occurring protein amino acids, which are referred to by both their common three letter abbreviation and single letter abbreviation. See generally Synthetic Peptides: A User's Guide, G. A. Grant, editor, W.H. Freeman & Co., New York (1992), the teachings of which are incorporated herein by reference, including the text and table set forth at pages 11 through 24. An “amino acid” includes conventional α-amino acids and further includes β-amino acids, α,α-disubstituted amino acids and N-substituted amino acids wherein at least one side chain is an amino acid side chain moiety as defined herein. An “amino acid” further includes N-alkyl α-amino acids, wherein the N-terminus amino group has a C₁ to C₆ linear or branched alkyl substituent. It may thus be seen that the term “amino acid” includes stereoisomers and modifications of naturally occurring protein amino acids, non-protein amino acids, post-translationally modified amino acids, enzymatically synthesized amino acids, derivatized amino acids, constructs or structures designed to mimic amino acids, and the like. Modified and unusual amino acids are described generally in Synthetic Peptides: A Users Guide, cited above; Hruby V. J., Al-obeidi F., Kazmierski W., Biochem. J. 268:249-262 (1990); and Toniolo C., Int. J. Peptide Protein Res. 35:287-300 (1990); the teachings of all of which are incorporated herein by reference. In addition, the following abbreviations, including amino acids and protecting and modifying groups thereof, have the meanings given:
Abu—gamma-amino butyric acid
12-Ado—12-amino dodecanoic acid
Aib—alpha-aminoisobutyric acid
6-Ahx—6-amino hexanoic acid
Amc—4-(aminomethyl)-cyclohexane carboxylic acid
8-Aoc—8-amino octanoic acid
Bip—biphenylalanine
Boc—t-butoxycarbonyl
Bzl—benzyl
Bz—benzoyl
Cit—citrulline
Dab—diaminobutyric acid
Dap—diaminopropionic acid
Dip—3,3-diphenylalanine
Disc—1,3-dihydro-2H-isoindolecarboxylic acid
Et—ethyl
Fmoc—fluorenylmethoxycarbonyl
Hept—heptanoyl (CH₃—(CH₂)₅—C(═O)—)
Hex—hexanoyl (CH₃—(CH₂)₄—C(═O)—)
HArg—homoarginine
HCys—homocysteine
HLys—homolysine
HPhe—homophenylalanine
HSer—homoserine
Me—methyl
Met(O)—methionine sulfoxide
Met(O₂)—methionine sulfone
Nva—norvaline
Pgl—phenylglycine
Pr—propyl
Pr-i—isopropyl
Sar—sarcosine
Tle—tert-butylalanine
Z—benzyloxycarbonyl

In the listing of natriuretic peptide mimetics according to the present invention, conventional amino acid residues have their conventional meaning as given in Chapter 2400 of the Manual of Patent Examining Procedure Ninth Edition. Thus, “Nle” is norleucine; “Asp” is aspartic acid; His is histidine; “Arg” is arginine; “Trp” is tryptophan; “Lys” is lysine; “Gly” is glycine; “Pro” is proline; “Tyr” is tyrosine, “Ser” is serine and so on. All residues are in the L-isomer configuration unless the D-isomer is specified, as in “D-Ala” for D-alanine.

A single amino acid, including stereoisomers and modifications of naturally occurring protein amino acids, non-protein amino acids, post-translationally modified amino acids, enzymatically synthesized amino acids, derivatized amino acids, an α,α-disubstituted amino acid derived from any of the foregoing (i.e., an α,α-disubstituted amino acid wherein at least one side chain is the same as that of the residue from which it is derived), a β-amino acid derived from any of the foregoing (i.e., a β-amino acid which other than for the presence of a β-carbon is otherwise the same as the residue from which it is derived) and the like, including all of the foregoing, is sometimes referred to herein as a “residue.”

An “α,α-disubstituted amino acid” includes any α-amino acid having a further substituent in the α-position, which substituent may be the same as or different from the side chain moiety of the α-amino acid. Suitable substituents, in addition to the side chain moiety of the α-amino acid, include C₁ to C₆ linear or branched alkyl. Aib is an example of an α,α-disubstituted amino acid. While α,α-disubstituted amino acids can be referred to using conventional L- and D-isomeric references, it is to be understood that such references are for convenience, and that where the substituents at the α-position are different, such amino acid can interchangeably be referred to as an α,α-disubstituted amino acid derived from the L- or D-isomer, as appropriate, of a residue with the designated amino acid side chain moiety. Thus (S)-2-Amino-2-methyl-hexanoic acid can be referred to as either an α,α-disubstituted amino acid derived from L-Nle or as an α,α-disubstituted amino acid derived from D-Ala. Whenever an α,α-disubstituted amino acid is provided, it is to be understood as including all (R) and (S) configurations thereof.

An “N-substituted amino acid” includes any amino acid wherein an amino acid side chain moiety is covalently bonded to the backbone amino group, optionally where there are no substituents other than H in the α-carbon position. Sarcosine is an example of an N-substituted amino acid. By way of example, sarcosine can be referred to as an N-substituted amino acid derivative of Ala, in that the amino acid side chain moiety of sarcosine and Ala is the same, methyl.

The term “amino acid surrogate” includes a molecule disclosed herein which is a mimic of a residue, including but not limited to piperazine core molecules, keto-piperazine core molecules and diazepine core molecules. Unless otherwise specified, an amino acid surrogate is understood to include both a carboxyl group and amino group, and a group corresponding to an amino acid side chain, or in the case of an amino acid surrogate of glycine, no side chain other than hydrogen. Thus an amino acid surrogate includes a molecule of the general formula of formula I or II given above. An amino acid surrogate further includes molecules of any of the following structures, it being understood that for convenience such structures are given as the isolated surrogate, not including any protecting group and not bound by one or two peptide bonds to one or two amino acid residues forming a part of a mimetic employed in the practice of the invention:

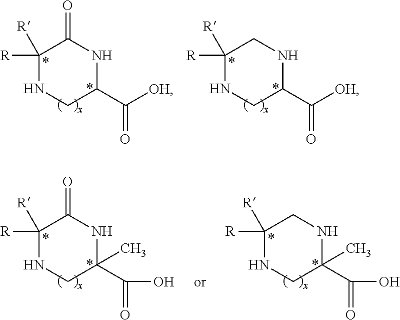

where R, R′, x and the asterisks are as defined for the surrogate of formula I. An amino acid surrogate further includes molecules of any of the following structures, again it being understood that for convenience such structures are given as the isolated surrogate, not including any protecting group and not bound by one or two peptide bonds to one or two amino acid residues forming a part of a mimetic employed in the practice of the invention:

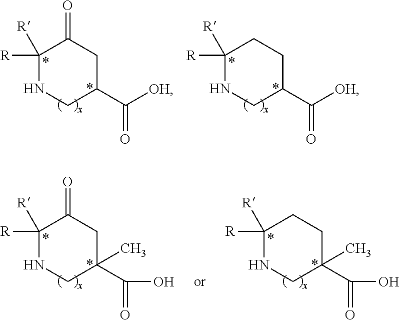

where R, R′, x and the asterisks are as defined for the surrogate of formula I. For purposes of synthesis, either the carboxyl group or the amino group of any amino acid surrogate is preferably protected by a protecting group, such that it is not reactive while the protecting group is present, and similarly any reactive group forming a part of R or R′ may similarly be protected by a protecting group. It will be appreciated that the surrogates of the present invention have more than one asymmetric center, and therefore are capable of existing in more than one stereoisomeric form. Some of the compounds may also exist as geometric isomers and rotamers. Furthermore, some compounds of the invention may also have conformational axial chirality resulting in atropisomers. The invention extends to each of these forms individually and to mixtures thereof, including racemates. In one aspect, surrogate isomers may be separated conventionally by chromatographic methods or by use of a resolving agent. In another aspect, individual surrogate isomers, or enantiomerically pure surrogates, are prepared by synthetic schemes, such as those disclosed herein or variants of such schemes, employing asymmetric synthesis using chiral intermediates, reagents or catalysts.

The term “C-terminus capping group” includes any terminal group attached through the terminal ring carbon atom or, if provided, terminal carboxyl group, of the C-terminus of a mimetic. The terminal ring carbon atom or, if provided, terminal carboxyl group, may form a part of a residue, or may form a part of an amino acid surrogate. In a preferred aspect, the C-terminus capping group forms a part of an amino acid surrogate which is at the C-terminus position of the mimetic. The C-terminus capping group includes, but is not limited to, —(CH₂)ₙ—OH, —(CH₂)ₙ—C(═O)—OH, —(CH₂)ₘ—OH, —(CH₂)ₙ—C(═O)—N(v₁)(v₂), —(CH₂)ₙ—C(═O)—(CH₂)ₘ—N(v₁)(v₂), —(CH₂)ₙ—O—(CH₂)ₘ—CH₃, —(CH₂)ₙ—C(═O)—NH—(CH₂)ₘ—CH₃, —(CH₂)ₙ—C(═O)—NH—(CH₂)ₘ—N(v₁)(v₂), —(CH₂)ₙ—C(═O)—N—((CH₂)ₘ—N(v₁)(v₂))₂, —(CH₂)ₙ—C(═O)—NH—CH(—C(═O)—OH)—(CH₂)ₘ—N(v₁)(v₂), —C(═O)—NH—(CH₂)ₘ—NH—C(═O)—CH(N(v₁)(v₂))((CH₂)ₘ—N(v₁)(v₂)), or —(CH₂)ₙ—C(═O)—NH—CH(C(═O)—NH₂)—(CH₂)ₘ—N(v₁)(v₂), including all (R) or (S) configurations of the foregoing, where v₁ and v₂ are each independently H, a C₁ to C₁₇ linear or branched alkyl chain, m is 0 to 17 and n is 0 to 2; or any omega amino aliphatic, terminal aryl or aralkyl, including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, or any single natural or unnatural α-amino acid, 8-amino acid or α,α-disubstituted amino acid, including all (R) or (S) configurations of the foregoing, optionally in combination with any of the foregoing non-amino acid capping groups. In the foregoing, it is to be understood that, for example, —C(═O)—NH—(CH₂)ₘ—NH—C(═O)—CH(N(v₁)(v₂))((CH₂)ₘ—N(v₁)(v₂)) is:

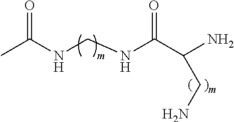

The term “N-terminus capping group” includes any terminal group attached through the terminal amine of the N-terminus of a mimetic. The terminal amine may form a part of a residue, or may form a part of an amino acid surrogate. In a preferred aspect, the N-terminus capping group forms a part of an amino acid surrogate which is at the N-terminus position of the mimetic. The N-terminus capping group includes, but is not limited to, any omega amino aliphatic, acyl group or terminal aryl or aralkyl including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, or alternatively an N-terminus capping group

is —(CH₂)ₘ—NH(v₃), —(CH₂)ₘ—CH₃, —C(═O)—(CH₂)ₘ—CH₃, —C(═O)—(CH₂)ₘ—NH(v₃), —C(═O)—(CH₂)ₘ—C(═O)—OH, —C(═O)—(CH₂)ₘ—C(═O)-(v₄), —(CH₂)ₘ—C(═O)—OH, —(CH₂)ₘ—C(═O)-(v₄), —C(═O)—(CH₂)ₘ—O(v₃), —(CH₂)ₘ—O(v₃), C(═O)—(CH₂)ₘ—S(v₃), or —(CH₂)ₘ—S(v₃), where v₃ is H or a C₁ to C₁₇ linear or branched alkyl chain, and v₄ is a C₁ to C₁₇ linear or branched alkyl chain and m is 0 to 17.

A phenyl ring is “substituted” when the phenyl ring includes one or more substituents independently comprising hydroxyl, halogen, alkyl, or aryl groups attached directly or through an ether linkage. Where the phenyl ring is so substituted, the amino acid residue may be referred to as substituted, as in substituted Phe, substituted HPhe or substituted Pgl.

The term “alkene” includes unsaturated hydrocarbons that contain one or more double carbon-carbon bonds. Examples of alkene groups include ethylene, propene, and the like.

The term “alkenyl” includes a linear monovalent hydrocarbon radical of two to six carbon atoms or a branched monovalent hydrocarbon radical of three to six carbon atoms containing at least one double bond; examples thereof include ethenyl, 2-propenyl, and the like.

The “alkyl” groups specified herein include those alkyl radicals of the designated length in either a straight or branched configuration. Examples of alkyl radicals include methyl, ethyl, propyl, isopropyl, butyl, sec-butyl, tertiary butyl, pentyl, isopentyl, hexyl, isohexyl, and the like.

The term “alkynyl” includes a linear monovalent hydrocarbon radical of two to six carbon atoms or a branched monovalent hydrocarbon radical of three to six carbon atoms containing at least one triple bond; examples thereof include ethynyl, propynal, butynyl, and the like.

The term “aryl” includes a monocyclic or bicyclic aromatic hydrocarbon radical of 6 to 12 ring atoms, and optionally substituted independently with one or more substituents selected from alkyl, haloalkyl, cycloalkyl, alkoxy, alkythio, halo, nitro, acyl, cyano, amino, monosubstituted amino, disubstituted amino, hydroxy, carboxy, or alkoxy-carbonyl. Examples of aryl groups include phenyl, biphenyl, naphthyl, 1-naphthyl, and 2-naphthyl, derivatives thereof, and the like.

The term “aralkyl” includes a radical —R^{a}R^{b} where R^{a} is an alkylene (a bivalent alkyl) group and R^{b} is an aryl group as defined above. Examples of aralkyl groups include benzyl, phenylethyl, 3-(3-chlorophenyl)-2-methylpentyl, and the like.

The term “aliphatic” includes compounds with hydrocarbon chains, such as for example alkanes, alkenes, alkynes, and derivatives thereof.

The term “acyl” includes a group R—C(═O)—, where R is an organic group. An example is the acetyl group CH₃—C(═O)—, referred to herein as “Ac”.

A peptide or aliphatic moiety is “acylated” when an aryl, alkyl or substituted alkyl group as defined above is bonded through one or more carbonyl {—(C═O)—} groups. A peptide is most usually acylated at the N-terminus.

An “omega amino aliphatic” includes an aliphatic moiety with a terminal amino group. Examples of omega amino aliphatics include 7′-amino-heptanoyl and the amino acid side chain moieties of ornithine and lysine.

The term “heteroaryl” includes mono- and bicyclic aromatic rings containing from 1 to 4 heteroatoms selected from nitrogen, oxygen and sulfur. 5- or 6-membered heteroaryl are monocyclic heteroaromatic rings; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine.

An “amide” includes compounds that have a trivalent nitrogen attached to a carbonyl group (—C(═O)—NH₂), such as for example methylamide, ethylamide, propylamide, and the like.

An “imide” includes compounds containing an imido group (—C(═O)—NH—C(═O)—). An “amine” includes compounds that contain an amino group (—NH₂).

A “nitrile” includes compounds that are carboxylic acid derivatives and contain a (—CN) group bound to an organic group.

The term “halogen” is intended to include the halogen atoms fluorine, chlorine, bromine and iodine, and groups including one or more halogen atoms, such as —CF₃ and the like.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a natriuretic peptide mimetic employed in the practice of the present invention and a pharmaceutically acceptable carrier.

The term “EC₅₀” is intended to include the molar concentration of an agonist which produced 50% of the maximum possible response for that agonist. By way of example, a natriuretic peptide mimetic which, at a concentration of 72 nM, produces 50% of the maximum possible response for that mimetic as determined in a cGMP assay, has an EC₅₀ of 72 nM. Unless otherwise specified, the molar concentration associated with an EC₅₀ determination is in nanomoles (nM).

The term “Ki (nM)” is intended to include the equilibrium receptor binding affinity representing the molar concentration of a competing compound that binds to half the binding sites of a receptor at equilibrium in the absence of a competitor. In general, the Ki is inversely correlated to the affinity of the compound for the receptor, such that if the Ki is low, the affinity is high. Ki may be determined using the equation of Cheng and Prusoff (Cheng Y., Prusoff W. H., Biochem. Pharmacol. 22: 3099-3108, 1973):

$$Ki = \frac{{EC}_{50}}{1 + \frac{[ ligand ]}{{K}_{d}}}$$

where “ligand” is the concentration of ligand, which may be a radioligand, and K_{d} is an inverse measure of receptor affinity which produces 50% receptor occupancy. Unless otherwise specified, the molar concentration associated with a Ki determination is nM.

By “a functional active ANP₉₉₋₁₂₆ deficiency” is meant less than optimal or desirable levels of NPRA agonism, such as less than optimal or desirable levels of active ANP₉₉₋₁₂₆, in a subject. In one aspect, less than optimal or desirable levels of active ANP₉₉₋₁₂₆ is determined by reference to average values for a normal control or cohort of normal controls. In another aspect, less than optimal or desirable levels of active ANP₉₉₋₁₂₆ is determined by reference to the ratio of total ANP, or some surrogate for total ANP such as pro-ANP, relative to ANP₉₉₋₁₂₆, wherein increased total ANP (or a surrogate therefore) relative to ANP₉₉₋₁₂₆ is indicative of less than optimal or desirable levels of active ANP₉₉₋₁₂₆. Whether the ratio of total ANP (or a surrogate therefore) is increased relative to ANP₉₉₋₁₂₆ may be determined by reference to corresponding average values for a normal control or cohort of normal controls. In another aspect, less than optimal or desirable levels of active ANP₉₉₋₁₂₆ is determined by reference to either absolute levels of cGMP or the level of cGMP by reference to corresponding average values for a normal control or cohort of normal controls. In yet another aspect, less than optimal or desirable levels of active ANP₉₉₋₁₂₆ is determined by reference to the disease state of the subject, wherein the patient has less than optimal or desirable levels of active ANP₉₉₋₁₂₆ if the disease condition of the subject is improved, ameliorated or stabilized by administration of a compound resulting in increased NPRA agonism, such as where the disease condition is cardiovascular disease, including but not limited to hypertension, acute coronary syndrome, cardiomyopathy, cardiac remodeling, left-ventricular hypertrophy, congestive heart failure, heart failure, high blood pressure and coronary artery disease.

By the term “biological sample” is meant a sample of biological tissue or fluid that comprises the desired analyte. Thus where nucleic acids are to be detected, the biological sample comprises nucleic acids. Where natriuretic peptides are to be detected, the biological sample comprises natriuretic peptides. Such samples include, but are not limited to, tissue isolated from a human. Thus biological samples may include tissue sections such as biopsy and autopsy samples, frozen sections or samples, blood, plasma, serum, sputum, stool, tears, mucus, hair and skin.

The chemical naming protocol and structure diagrams used herein employ and rely on the chemical naming features as utilized by the ChemDraw program (available from Cambridgesoft Corp., Cambridge, Mass.). In particular, certain compound names were derived from the structures using the Autonom program as utilized by Chemdraw Ultra or ISIS base (MDL Corp.). In general, structure diagrams do not depict hydrogen atoms associated with carbon atoms other than in terminal groups and other special circumstances.

Certain structure diagrams and drawings herein depict natriuretic peptide mimetics composed of amino acid surrogates and amino acid residues, with the surrogates identified by structure diagrams and the amino acid residues identified by a three letter abbreviation. Unless otherwise specified, it is understood that the bond between the surrogate and residue, or between the residue and surrogate, or between a surrogate and residues on both the N-terminus and C-terminus side thereof, is a conventional peptide bond, —C(═O)—NH—or, in the case where the peptide bond is to the ring nitrogen on the N-terminus of the surrogate, —C(═O)—N—. In general, in the depiction of such bonds the atoms of the amino acid surrogate are depicted (e.g., —C(═O)— or —N), but atoms of the amino acid residue are not depicted.

Clinical Applications.

The methods of use of natriuretic peptide mimetics disclosed herein can be used for both medical applications and animal husbandry or veterinary applications. Typically, the mimetic, or a pharmaceutical composition including the mimetic, is used in humans, but may also be used in other mammals. The term “patient” is intended to denote a mammalian individual, and is so used throughout the specification and in the claims. The primary applications of this invention involve human patients, but this invention may be applied to laboratory, farm, zoo, wildlife, pet, sport or other animals.

The methods and uses disclosed herein may be employed for the prophylaxis or treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency.

In one aspect, the invention relates to a method for reducing cardiac remodeling in a patient identified as being in need thereof, including in a patient with a functional natriuretic peptide deficiency, which method comprises administering to the patient a composition comprising a pharmaceutically acceptable carrier and a natriuretic peptide mimetic capable of increasing urinary and plasma cGMP levels in the patient. Preferably the composition is administered in an amount effective to alter the level of one or more parameters of cardiac remodeling by at least ten percent as compared to levels of the one or more parameters prior to administration of the composition. The one or more parameters can include cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure.

In another aspect, the invention relates to natriuretic peptide mimetics and compositions including mimetics that can be used in a patient with a functional natriuretic peptide deficiency to increase cGMP levels, and preferably thereby to halt, reduce or reduce the rate of cardiac remodeling. The mimetics can bind to an NPR, including one or more of NPRA, NPRB and NPRC.

In some embodiments, reduced cardiac remodeling can be indicated by one or more parameters including cardiac unloading (i.e., reduced pressure in the heart), increased glomerular filtration rate (GFR), decreased plasma renin activity (PRA), decreased levels of angiotensin II, decreased proliferation of cardiac fibroblasts, decreased left ventricular (LV) hypertrophy, decreased LV mass (indicative of reduced fibrosis and hypertrophy), decreased pulmonary wedge capillary pressure (PWCP; an indirect measure of left atrial pressure), decreased right atrial pressure, decreased mean arterial pressure, decreased levels of aldosterone (indicative of an anti-fibrotic effect), decreased ventricular fibrosis, increased ejection fraction, and decreased LV end systolic diameter. One or more of these parameters can be evaluated, such as before and after a set period of treatment with a natriuretic peptide mimetic or composition including a mimetic, to determine whether the mimetic is inhibiting or reducing cardiac remodeling. Such parameters may be evaluated by methods known in the art, including methods described herein.

A functional natriuretic peptide deficiency may arise in a patient from any cause. In one aspect, there is a deficiency, omission, or inefficiency in one or more steps of processing a natriuretic peptide to yield a functional or active natriuretic peptide. Thus differential expression of the pro-natriuretic peptide convertases corin and furin has been observed in animal models, including canine models, with decreases in corin mRNA and protein in experimental heart failure and atrial fibrosis and increases in furin mRNA and protein expressions. Ichiki, T. et al.: “Differential expression of the pro-natriuretic peptide convertases corin and furin in experimental heart failure and atrial fibrosis,” Am J Physiol Regul lntegr Comp Physiol 304:R102-R109 (2013).

It is known that adverse cardiovascular outcomes, such as decompensated heart failure, are associated with both reduced corin levels and decreased cleavage of pro-atrial natriuretic peptide, both of which result in a functional active ANP₉₉₋₁₂₆ deficiency. Ibebuogu, U. N. et al.: “Decompensated heart failure is associated with reduced corin levels and decreased cleavage of pro-atrial natriuretic peptide,” Circ Heart Fail 4:113-120 (2011).

One processing mutation, variation or polymorphism that gives rise to a functional natriuretic peptide deficiency is a corin mutation, variation or polymorphism. Corin mutations are known to affect specific populations. There are two nonsynonymous, nonconservative single nucleotide polymorphisms (Q568P and T555I) in near-complete linkage disequilibrium, thus describing a single minor I555 (P568) corin gene allele. This allele is present in the heterozygote state in approximately 12% of persons of African ancestry but is extremely rare in whites. Dries, D.; Victor, R. G. et al.: “Corin gene minor allele defined by 2 missense mutations is common in blacks and associated with high blood pressure and hypertension,” Circulation 112:2403-2410 (2005). The allele is associated with a functional active ANP₉₉₋₁₂₆ deficiency and increased risk for hypertension and other cardiovascular effects. The two single nucleotide polymorphisms result in a threonine to isoleucine substitution at amino acid position 555 (T555I), and glutamic acid to proline substitution at amino acid position 568 (Q568P), both in the Frizzled2 (Fz2) domain, an α-helix on the domain surface of corin. In functional experiments, the allele results in corin with a reduced activity in processing pro-ANP, resulting in a functional active ANP₉₉₋₁₂₆ deficiency. The allele is associated with an increased risk for death or heart failure hospitalization. Rame, J. E. et al.: “Dysfunctional corin I555 (P568) allele is associated with impaired BNP processing and adverse outcomes in African-Americans with systolic heart failure: results from the genetic risk assessment in hear failure A-HeFT sub-study,” Circ Heart Fail 2:541-548 (2009).

Another corin mutation is the R539C mutation, which causes an arginine to cysteine substitution at residue 539 in the Fz2 domain. The mutation is a C→T heterozygous mutation at nucleotide position 1708 in exon 12. Dong, N. et al.: “Corin mutation R539C from hypertensive patients impairs zymogen activation and generates inactive alternative ectodomain fragment,” J. Biol. Chem. 288:7867-74 (2013). The R539C mutation may be detected by standard techniques, including PCR amplification with direct sequencing by a DNA analyzer. The R539C mutation, which has been detected only in ethnic Han Chinese, results in creased blood pressure and other cardiovascular effects. In functional experiments, the R539C mutation impairs corin zymogen activation, and has a reduced activity in processing pro-ANP, resulting in a functional active ANP₉₉₋₁₂₆ deficiency. Ibebuogu, U. N. et al.: “Decompensated heart failure is associated with reduced corin levels and decreased cleavage of pro-atrial natriuretic peptide,” Circ Heart Fail 4:113-120 (2011).

Corin, and corin mutations, may be detected by antibodies that are specific for corin or a portion thereof, including antibodies that selectively bind to corin but do not substantially bind to other molecules in a biological sample. It is possible and contemplated that antibodies may be specific for corin generally and may also be specific for corin mutations, or conversely, or corin not exhibiting a mutation. It is also possible that corin nucleic acid or portions thereof can be detected. For example, nucleic acid may be isolated using conventional methods, such as lytic enzymes or other chemical solutions, and the mRNA of a gene in the isolated nucleic acid can then be detected, such as a Northern blot analysis to detect hybridization, optionally with amplification to facilitate detection, such as amplification by means of a polymerase chain reaction. Various nucleic acid probes can be used, including problems exhibiting sequence complementarity or homology to corin or a portion thereof. The methods and teaching of WO 2010/096658, entitled “Corin as a marker for heart failure”; and Yan, W.; Wu, F. et al.: “Corin, a transmembrane cardiac serine protease, acts as a pro-atrial natriuretic peptide-converting enzyme,” Proc Natl Acad Sc. USA 97:8525-8529 (2000), are incorporated herein by reference. A deficit or deficiency in active ANP₉₉₋₁₂₆ may result from mutations in the gene coding for human corin: Wang, W.; Liao, X. et al.: “Corin variant associated with hypertension and cardiac hypertrophy exhibits impaired zymogen activation and natriuretic peptide processing activity,” Circ Res 103:502-508 (2008); and Dries, D.; Victor, R. G. et al.: “Corin gene minor allele defined by 2 missense mutations is common in blacks and associated with high blood pressure and hypertension,” Circulation 112:2403-2410 (2005).

It is also possible to have a genetic mutation, variation or polymorphism, including a single nucleotide polymorphism (SNP), in the expression of ANP, including ANP₉₉₋₁₂₆, resulting in a variation of ANP, including a variation of ANP₉₉₋₁₂₆, which may result in ANP with reduced or altered efficacy, and may further result in increased susceptibility to disease. In one aspect, a subject is homozygous for the major “A” allele (e.g., is AA) of the SNP rs5068 (A/G). In another aspect, a subject is heterozygous for the major “A” allele (e.g., is AG) of the SNP rs5068 (A/G). The SNP rs5068 (A/G) is located in the 3′ untranslated region (UTR) of the NPPA gene, which encodes the ANP propeptide. A minor (G) allele of rs5068 affects plasma ANP levels, and absence of this minor variant (G), such as subjects homozygous for the major A allele (e.g., AA subjects) or heterozygous for the major A allele (e.g., AG subjects), have lower levels of ANP. Thus by rs5068 genotype, the effect of the genotype (e.g., AA or AG) of rs5068 on subjects' ANP levels and physiologic response to salt may be predicted. In this context, the teachings of WO 2013/188787, entitled “Inhibitors of microRNAs that regulate production of atrial natriuretic peptide (ANP) as therapeutics and uses thereof,” including the methods and references cited therein, are incorporated herein by reference.

It is also possible and contemplated that a functional active ANP₉₉₋₁₂₆ deficiency may have an unknown etiology. Even if the etiology is unknown, the patient with a functional active ANP₉₉₋₁₂₆ deficiency may nonetheless be treated by the methods of the invention disclosed herein, including treatment administration of any natriuretic peptide mimetic that binds to and activates NPRA, including but not limited to the natriuretic peptide mimetic of formula IV.

It has been reported that high levels, often very high levels, of ANP occur in patients with severe heart failure or severe cardiovascular disease. Thus it has been assumed that high levels of total ANP do not result in improved outcomes. However, it is now known that current commonly used immunoassays cannot accurately measure or distinguish among pro-ANP, NT-ANP, urodilatin, and ANP₉₉₋₁₂₆. This is because, in part, most immunoassays utilize antibodies that are directed to either the N-terminal or C-terminal portion of ANP variants. Thus immunoassays directed against C-terminal sequences in ANP detect both pro-ANP and ANP₉₉₋₁₂₆. There are assay systems which have been developed which discriminate between various forms of ANP, and such assay systems may be utilized in the practice of this invention. In one such assay system, an immunodepletion method is employed, with the result that levels of active ANP₉₉₋₁₂₆ are detected. In this context, the methods and teachings of Ibebuogu, U. N. et al.: “Decompensated heart failure is associated with reduced corin levels and decreased cleavage of pro-atrial natriuretic peptide,” Circ Heart Fail 4:113-120 (2011) are incorporated herein by reference. By means of immunodepletion, it is possible to measure ANP₉₉₋₁₂₆ to the exclusion of other forms of ANP, such as pro-ANP, NT-ANP and the like. Other methods and assay systems for detecting ANP₉₉₋₁₂₆ and precursor fragments, such as pro-ANP, are disclosed in Goetze, J. P. et al.: “Atrial natriuretic peptides in plasma,” Clinica Chimica Acta 443:25-29 (2015), incorporated herein by reference.

It may readily be seen that a functional active ANP₉₉₋₁₂₆ deficiency may be determined in any number of ways, including without limitation: (a) testing for active ANP₉₉₋₁₂₆; (b) testing for both active ANP₉₉₋₁₂₆ and a precursor, such as pro-ANP and determining the ratios thereof; (c) testing for a genetic mutation, variation or polymorphism, including a SNP, in the expression of ANP, including ANP₉₉₋₁₂₆; (d) testing for a deleterious mutation, variation or polymorphism in expressed ANP, including ANP₉₉₋₁₂₆, such as one or more amino acid substitutions, deletions or insertions; (e) testing for a genetic mutation, variation or polymorphism, including a SNP, in the expression of a protein required for processing ANP, such as in the expression of corin; (f) testing for a deleterious mutation, variation or polymorphism in expressed protein required for processing ANP, such as a mutation, variation or polymorphism in corin which is one or more amino acid substitutions, deletions or insertions; (g) testing for levels in cyclic guanosine monophosphate (cGMP) in plasma or another biological sample, where a decreased level is correlated with a functional active ANP₉₉₋₁₂₆ deficiency; (h) by the symptoms of the patient, including specifically symptoms consistent with a functional active ANP₉₉₋₁₂₆ deficiency; or (i) by other means by which a functional active ANP₉₉₋₁₂₆ deficiency may be determined, detected or inferred.

Treatment and Prophylaxis of Disease.

In one aspect, there is provided a method for the treatment of cardiovascular disease, including but not limited to hypertension, acute coronary syndrome, cardiomyopathy, cardiac remodeling, left-ventricular hypertrophy, congestive heart failure, heart failure, high blood pressure and coronary artery disease, in patients with a defect, condition, syndrome, disease or mutation resulting in a functional active ANP₉₉₋₁₂₆ deficiency. This method may be employed to treat cardiovascular disease which is not responsive, or not adequately responsive, to treatment with existing drugs, such as cardiovascular disease that is not response, or not adequately response, to treatment with one or more antihypertensive drugs such as diuretics, adrenergic receptor antagonists, calcium channel blockers, renin inhibitors, angiotensin-converting enzyme inhibitors, angiotensin II receptor antagonists, aldosterone receptor antagonists, vasodilators, α₂ agonists or endothelin receptor blockers. Alternatively, the methods of this invention, including the uses of the natriuretic peptide mimetics herein, may be used to treat cardiovascular disease without regard to whether the cardiovascular disease is responsive, or adequately responsive, to treatment with existing drugs.

In another aspect, there is provided a method for the prevention or prophylaxis of disease in patients with a defect, condition, syndrome, disease or mutation resulting in a functional active ANP₉₉₋₁₂₆ deficiency, which method includes the administration of any natriuretic peptide mimetic that binds to and activates one or more NPR, including NPRA, such that the functional level of active ANP₉₉₋₁₂₆, if any is present, together with mimetic, results in a level of NPR agonism, including NPRA agonism, corresponding to an optimal or desirable level of NPR, including NPRA, activation, thereby preventing, ameliorating, reducing the effects of or delaying the onset of cardiovascular disease, including but not limited to hypertension, acute coronary syndrome, cardiomyopathy, cardiac remodeling, left-ventricular hypertrophy, congestive heart failure, heart failure, high blood pressure and coronary artery disease.

Salt Form of Mimetics.

The natriuretic peptide mimetics employed in this invention may be in the form of any pharmaceutically acceptable salt. The term “pharmaceutically acceptable salts” refers to salts prepared from pharmaceutically acceptable non-toxic bases or acids including inorganic or organic bases and inorganic or organic acids. Salts derived from inorganic bases include salts of aluminum, ammonium, calcium, copper, ferric, ferrous, lithium, magnesium, manganic, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the natriuretic peptide mimetic employed in the present invention is basic, acid addition salts may be prepared from pharmaceutically acceptable non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Acid addition salts of natriuretic peptide mimetics are prepared in a suitable solvent from the mimetic and an excess of an acid, such as hydrochloric, hydrobromic, sulfuric, phosphoric, acetic, trifluoroacetic, citric, tartaric, maleic, succinic or methanesulfonic acid. The acetate salt form is especially useful. Where the mimetics of embodiments of this invention include an acidic moiety, suitable pharmaceutically acceptable salts may include alkali metal salts, such as sodium or potassium salts, or alkaline earth metal salts, such as calcium or magnesium salts.

In addition, Applicant has advantageously discovered that certain salt forms of the natriuretic peptide mimetics employed in this invention, including pamoate, octanoate, decanoate, oleate, stearate, sodium tannate and palmitate salt forms, have an mean residence time half-life, in some cases doubled, versus the corresponding acetate salt form. These salt forms are particularly well-suited for administration by subcutaneous injection or intramuscular injection, especially for chronic treatment, due to the reduced frequency of dosing that may be achieved as a result of the longer half-lives. While not being limited by theory, it is believed the increased half-life is related to a decrease in solubility in comparison to the acetate salt form. The increased half-life salt forms of the natriuretic peptide mimetics of the invention may be manufactured by any method including, for example, ion exchange, mixing a solution of an acetate salt form of a mimetic with disodium pamoate to form a pamoate suspension, or use of the desired salt during the final purification step(s) in the manufacture of the mimetics.

Pharmaceutical Compositions.

The present invention may utilize a pharmaceutical composition that includes a natriuretic peptide mimetic and a pharmaceutically acceptable carrier. The carrier may be a liquid formulation, and is preferably a buffered, isotonic, aqueous solution. Pharmaceutically acceptable carriers also include excipients, such as diluents, carriers and the like, and additives, such as stabilizing agents, preservatives, solubilizing agents, buffers and the like, as hereafter described.

The natriuretic peptide mimetics may be formulated or compounded into pharmaceutical compositions that include at least one mimetic together with one or more pharmaceutically acceptable carriers, including excipients, such as diluents, carriers and the like, and additives, such as stabilizing agents, preservatives, solubilizing agents, buffers and the like, as may be desired. Formulation excipients may include polyvinylpyrrolidone, gelatin, hydroxy cellulose, acacia, polyethylene glycol, manniton, sodium chloride and sodium citrate. For injection or other liquid administration formulations, water containing at least one or more buffering constituents is preferred, and stabilizing agents, preservatives and solubilizing agents may also be employed. For solid administration formulations, any of a variety of thickening, filler, bulking and carrier additives may be employed, such as starches, sugars, fatty acids and the like. For most pharmaceutical formulations, non-active ingredients will constitute the greater part, by weight or volume, of the preparation. For pharmaceutical formulations, it is also contemplated that any of a variety of measured-release, slow-release or time-release formulations and additives may be employed, so that the dosage may be formulated so as to effect delivery of a natriuretic peptide mimetic over a period of time. For example, gelatin, sodium carboxymethylcellulose and/or other cellulosic excipients may be included to provide time-release or slower-release formulations, especially for administration by subcutaneous and intramuscular injection.

In general, the actual quantity of natriuretic peptide mimetic administered to a patient will vary between fairly wide ranges depending on the mode of administration, the formulation used, and the response desired.

In practical use, the natriuretic peptide mimetics can be combined as the active ingredient in an admixture with a pharmaceutical carrier according to conventional pharmaceutical compounding techniques. The carrier may take a wide variety of forms depending on the form of preparation desired for administration, for example, oral, parenteral (including intravenous), urethral, vaginal, nasal, dermal, transdermal, pulmonary, deep lung, inhalation, buccal, sublingual, or the like. In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets.

Formulations specific for natriuretic peptide compositions may be employed in the practice of this invention. For example, the therapeutic compositions disclosed in International Publication No. WO 2013/016148 A2, published Jan. 31, 2013, and incorporated herein by reference, may be employed. Thus the natriuretic peptide mimetics employed in the practice of this invention may be in a composition comprising one or more of tris(hydroxymethyl)aminomethane and a phosphate buffer, meta-cresol, and water.

Natriuretic peptide mimetics may be administered parenterally. Solutions or suspensions of these active peptides may be prepared in water suitably mixed with a surfactant such as hydroxy-propylcellulose. Dispersions may also be prepared in glycerol, liquid polyethylene glycols and mixtures thereof in oils. These preparations may optionally contain a preservative to prevent the growth of microorganisms. Lyophilized single unit formulations may also be utilized, which are reconstituted, such as with saline, immediately prior to administration, and thus do not require a preservative.

The pharmaceutical forms suitable for injectable use include, for example, sterile aqueous solutions or dispersions and sterile powders, such as lyophilized formulations, for the extemporaneous preparation of sterile injectable solutions or dispersions. In all cases, the form must be sterile and must be fluid to the extent that it may be administered by syringe. The form must be stable under the conditions of manufacture and storage and must be preserved against the contaminating action of microorganisms such as bacteria and fungi. The carrier can be a solvent or dispersion medium containing, for example, water, ethanol, a polyol, for example glycerol, propylene glycol or liquid polyethylene glycol, suitable mixtures thereof, and vegetable oils.

Any of a variety of sustained release formulations may be employed. In one aspect, any formulation that can be utilized with mimetics that have high aqueous solubility can be employed. Examples include emulsions, such as water-in-oil emulsion, various viscosity enhancing agents, including gels such as gematin or cellulose-based viscosity enhancing agents, suspension, such as oil-base or water insoluble salt form suspensions, and various other approaches, including use of liposomes and microspheres. In one aspect, a polymer gel composition is employed, such as a composition comprising poly(lactic-co-glycolic acid), N-methyl-2-pyrrolidinone and triacetin, as disclosed in U.S. Published Patent Application No. 2014/0357561 A1, published Dec. 4, 2014.

Applications and Uses.

In one embodiment, there is providing a method which includes administering an amount of an natriuretic peptide mimetic sufficient to inhibit, reduce or decrease progression, severity, frequency, probability, duration or prevent one or more adverse physiological or psychological symptoms caused by or associated with a functional active ANP₉₉₋₁₂₆ deficiency.

Invention treatment methods include providing a given subject with an objective or subjective improvement of the condition, disorder or disease, a symptom caused by or associated with the condition, disorder or disease, or the probability or susceptibility of a subject to the condition or a symptom caused by or associated with the condition, disorder or disease. In various embodiments, treatment reduces, decreases, inhibits, delays, eliminates or prevents the probability, susceptibility, severity, frequency, or duration of one or more symptoms caused by or associated with the condition, disorder or disease. In a particular aspect, a method inhibits, reduces or decreases the probability, severity, frequency or duration of a subject from being diagnosed with cardiovascular disease, including but not limited to hypertension, acute coronary syndrome, cardiomyopathy, cardiac remodeling, left-ventricular hypertrophy, congestive heart failure, heart failure, high blood pressure and coronary artery disease.

Candidate subjects for methods of the invention include mammals, such as humans. Candidate subjects for methods of the invention also include subjects that are in need of treatment, e.g., any subject that may benefit from a treatment. Candidate subjects for methods of the invention therefore include subjects that have or are at risk of having a defect, condition, syndrome, disease or mutation resulting in a functional active ANP₉₉₋₁₂₆ deficiency, including subjects that have or are at risk of having cardiovascular disease, including but not limited to hypertension, acute coronary syndrome, cardiomyopathy, cardiac remodeling, left-ventricular hypertrophy, congestive heart failure, heart failure, high blood pressure and coronary artery disease.

Thus in one aspect a patient may have a cardiovascular disease, condition or disorder. This includes, without limitation, disorders characterized by insufficient, undesired or abnormal cardiac function, such as hypertension, ischemic heart disease, hypertensive heart disease and pulmonary hypertensive heart disease, valvular disease, congenital heart disease, acute coronary syndrome, cardiomyopathy, cardiac remodeling, left-ventricular hypertrophy, congestive heart failure, high blood pressure and coronary artery disease. This includes, but is not limited to, patients with a defect, condition, syndrome, disease or mutation resulting in a functional active ANP₉₉₋₁₂₆ deficiency. Insufficient, undesired or abnormal cardiac function may arise from disease, including genetic defects or acquired disease, injury, aging or a combination thereof. Acute coronary syndrome, which is also called coronary artery disease, includes to any disease, condition or disorder caused by or resulting from undesired or abnormal cardiac function, such as ischemic heart disease, hypertensive heart disease and pulmonary hypertensive heart disease, valvular disease, congenital heart disease and, in general, any condition which leads to congestive heart failure.

Routes of Administration.

If a natriuretic peptide mimetic for prophylaxis or treatment of a functional active ANP₉₉₋₁₂₆ deficiency is systemically administered, it may be administered by any means known in the art. Thus administration may be by injection, and the injection may be intravenous, subcutaneous, intramuscular, intraperitoneal or other means known in the art, and may include a bolus injection, a continuous infusion or an intermittent infusion.

The natriuretic peptide mimetics employed in the practice of this invention may further be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which the mimetics are introduced across an epidermal layer of cells may be employed. Administration means may thus include administration through mucous membranes, buccal administration, oral administration, dermal administration, inhalation administration, pulmonary administration, nasal administration, urethral administration, vaginal administration, and the like.

Any of a variety of devices and apparatuses may be utilized to administer the natriuretic peptide mimetic employed in this invention by any means, including subcutaneous, intramuscular or intravenous administration. The devices and apparatuses may include an external or implantable drug delivery pump, an implanted, subcutaneous, or percutaneous vascular access port, a direct delivery catheter system, and a local drug-release device.

In one aspect, natriuretic peptide mimetics employed in this invention are administered by transdermal means, such as electrotransport provided in U.S. Patent Application No. 2007/0249988, by means of a transdermal patch such as provided in U.S. Pat. No. 8,652,511 and the patents and applications cited therein or by means of a transdermal drug delivery device having a microprotrusion such as provided in U.S. Pat. No. 8,663,155.

In one aspect, a natriuretic peptide mimetic employed in this invention is administered by means of a time release injectable formulation, such as a mimetic in a formulation with a PEG, poly(ortho ester) or PLGA polymer. In another aspect, a mimetic employed in this invention is administered by means infusion, such as for example by means of an automated delivery device providing subcutaneous delivery or infusion, either continuous or intermittent. Any of the foregoing methods and formulations are particularly applicable for treatment of chronic conditions or syndromes, including specifically a functional active ANP₉₉₋₁₂₆ deficiency, such that the functional level of active ANP₉₉₋₁₂₆, if any is present, together with a mimetic employed in the practice of this invention administered by either a time-release injectable formulation or infusion, results in a level of NPR activation, including NPRA agonism, corresponding to an optimal or desirable level of active ANP₉₉₋₁₂₆.

In another aspect, a natriuretic peptide mimetic employed in this invention is administered in a multimodal dosage regime comprising at least an initial dosage stage and at least one maintenance dosage stage, with the objective of achieving a target stead state blood plasma concentration of the mimetic utilized in the practice of this invention. In this regard, the teachings of International Publication No. WO 2014/138796 A1, published Sep. 18, 2014, are incorporated by reference.

In another aspect, a natriuretic peptide mimetic employed in this invention is administered by means of continuous intradermal administration, such as utilizing a microneedle array, optionally a microneedle array using a delivery pump. Continuous intradermal administration of a mimetic employed in this invention can be used to maintain in vivo concentrations of the mimetic above a critical efficacy threshold for an extended period of time, such as by monitoring and actively adjusting the delivery of a mimetic employed in the practice of this invention. The teachings of International Publication No. WO 2013/151766 A1, published Oct. 10, 2013, are incorporated by reference, including teachings relating to a drug provisioning component that employs an array of microneedles to deliver a composition containing a mimetic employed in this invention peptide to the dermis, optionally intradermal delivery using an infusion pump at a continuous rate to maintain a specified plasma concentration of the mimetic employed in the practice of this invention.

In another aspect, a natriuretic peptide mimetic employed in this invention is administered utilizing continuing subcutaneous administration utilizing systems and methods which maintain in vivo concentrations of the mimetic employed in the practice of this invention above a critical efficacy threshold for an extended period of time, such as by monitoring and actively adjusting the delivery of a mimetic employed in this invention. The teachings of U.S. Patent Publication No. 2014/0031787, published Jan. 30, 2014, are incorporated by reference, including teachings relating to a medical device having one or more sensors adapted to detect at least one physiologic parameter relating to any one of cardiac function or fluid status of a patient, a pump for delivering one or more mimetics employed in the practice of this invention to a patient, wherein the pump is controlled by a control system, wherein the control system applies an algorithm to data received from the one or more sensors, and the algorithm determines the need for the patient to have an increased or decreased amount of a mimetic employed in this invention. The control system may have a data aggregation device function or may have access to a data aggregation device function for receiving and storing data from the one or more sensors. The sensors may determine a physiological parameter selected from blood pressure, pulmonary artery pressure, left atrial pressure, central venous pressure, lung fluid volume, proteinuria, plasma renin, central venous pressure, right atrial pressure and cardiac output.

Therapeutically Effective Amount.

In general, in the methods and uses of this invention the actual quantity of a natriuretic peptide mimetic administered to a patient will vary between fairly wide ranges depending upon the pharmacokinetic and pharmacodynamics properties of the active mimetic, the mode of administration, the formulation used, and the response desired. The dosage for treatment is administration, by any of the foregoing means or any other means known in the art, of an amount sufficient to bring about the desired therapeutic effect. A therapeutically effective amount includes an amount of a mimetic or pharmaceutical composition that is sufficient to provide a level of NPR activation, including NPRA agonism, corresponding to an optimal or desirable level of active ANP₉₉₋₁₂₆ at an acceptable and reasonable benefit/risk ratio considering the disease, prognosis and alternatives. In another aspect, a “therapeutically effective amount” comprises mimetic or pharmaceutical composition sufficient to effect a therapeutically significant reduction in a symptom or clinical marker associated with a cardiovascular conditions, diseases or disorders.

In one aspect, a therapeutically effective amount is an amount that results in a therapeutically significant reduction in a symptom, such as a reduction of at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Such therapeutically significant reduction may be observed at one or more time points relevant to treatment of the cardiovascular condition, disease, or disorder, such as one month, two months, three months or four months following commencement of treatment. Measured or measurable parameters include clinically detectable markers of disease, such as elevated or depressed levels of a biological marker, as well as parameters related to a clinically accepted scale of symptoms or markers for a disease or disorder.

Methods of the invention can be practiced by administration or contact with any dose amount, frequency, delivery route or timing of a natriuretic peptide mimetic as disclosed herein. In particular embodiments, a subject is administered a mimetic as disclosed herein one, two, three, four or more times hourly, daily, biweekly, weekly, monthly or annually. In additional embodiments, an amount administered is about 0.00001 mg/kg, to about 10,000 mg/kg, about 0.0001 mg/kg, to about 1000 mg/kg, about 0.001 mg/kg, to about 100 mg/kg, about 0.01 mg/kg, to about 10 mg/kg, about 0.1 mg/kg, to about 1 mg/kg body weight, one, two, three, four, or more times per hour, day, biweekly, week, month or annually. In further embodiments, the amount administered is less than about 0.001 mg/kg, such as between about 0.0001 and 0.0005 mg/kg, administered one, two, three, four, or more times per hour, day, biweekly, week, month or annually.

It is also possible that different methods of administration may be employed in the treatment of a single patient. For example, an NPRA agonist may be administered by a first route, such as intravenous administration, for a first period of time, and thereafter administered by a second route, such as subcutaneous, for a second period of time.

In another aspect, the methods of invention include administration by means of a sustained release, time release or delayed released formulation (collectively, a sustained release formulation), and the amount administered is determined by reference to the potency of the mimetic, the release relate and other pharmacokinetic and pharmacodynamics parameters.

Combination Therapy.

It is also possible and contemplated that the methods and uses of this invention include use of natriuretic peptide mimetics and compositions including mimetics in combination with other drugs or agents. In certain embodiments, an effective amount of one or more mimetics is administered in combination with an effective amount of one or more therapies used for treatment or prophylaxis of cardiovascular disease, including but not limited to hypertension, acute coronary syndrome, cardiomyopathy, cardiac remodeling, left-ventricular hypertrophy, congestive heart failure, heart failure, high blood pressure or coronary artery disease.

In one aspect, natriuretic peptide mimetics and compositions including mimetics may be used in combination with antihypertensive drugs, some of which drugs are also used for heart failure and treatment of other cardiovascular disease. Such drugs may be administered together or separately, and if administered separately, may be administered by different routes of administration from administration of mimetics and compositions including mimetics, and may be administered on a different schedule and at different times than with administration of mimetics and compositions including mimetics. Such antihypertensive drugs include, by way of example only and not limitation, diuretics, including loop diuretics such as bumetanide, ethacrynic acid, furosemide and torsemide, thiazide diuretics such as epitizide, hydrochlorothiazide, chlorothiazide and bendroflumethiazide, thiazide-like diuretics such as indapamide, chlorthalidone and metolazone, and potassium-sparing diuretics such as amiloride, triamterene and spironolactone; adrenergic receptor antagonists, including beta blockers such as atenolol, metoprolol, nadolol, nebivolol, oxprenolol, pindolol, propranolol and timolol, alpha blockers such as doxazosin, phentolamine, indoramin, henoxybenzamine, prazosin, terazosin and tolazoline, and mixed alpha and beta blockers such as bucindolol, carvedilol and labetalol; calcium channel blockers, including dihydropyridines such as amlodipine, cilnidipine, felodipine, isradipine, lercanidipine, levamlodipine, nicardipine, nifedipine, nimodipine and nitrendipine, and non-dihydropyridines such as diltiazem and verapamil; renin inhibitors such as aliskirin; angiotensin-converting enzyme (ACE) inhibitors such as captopril, enalapril, fosinopril, lisinopril, perindopril, quinapril, imidapril, ramipril, trandolapril and benazepril; angiotensin II receptor antagonists such as candesartan, eprosartan, irbesartan, losartan, olmesartan, telmisartan and valsartan; aldosterone receptor antagonists such as eplerenone and spironolactone; vasodilators such as sodium nitroprusside and hydralazine; α₂ agonists such as clonidine, guanabenz, guanfacine, methyldopa and moxonidine; and endothelin receptor blockers, such as bosentan.

In another aspect, natriuretic peptide mimetics and compositions including mimetics may be used in combination with statins, a class of drugs used to lower cholesterol levels by inhibiting the enzyme HMG-CoA reductase. Such drugs may be administered together or separately, and if administered separately, may be administered by different routes of administration from administration of mimetics and compositions including mimetics, and may be administered on a different schedule and at different times than with administration of mimetics and compositions including mimetics. Such statin drugs include, by way of example only and not limitation, atorvastatin, cerivastatin, fluvastatin, lovastatin, mevastatin, pitavastatin, pravastatin, rosuvastatin and simvastatin, as well as combinations of simvastatin and ezetimibe, lovastatin and niacin, atorvastatin and amlodipine, and simvastatin and niacin.

In another aspect, natriuretic peptide mimetics and compositions including mimetics may be used in combination with nitroglycerin, also known as glyceryl trinitrate. Nitroglycerin and mimetics and compositions including mimetics may be administered together or separately, and if administered separately, nitroglycerin may be administered by different routes of administration from administration of mimetics and compositions including mimetics, and may be administered on a different schedule and at different times than with administration of mimetics and compositions including mimetics.

In another aspect, natriuretic peptide mimetics and compositions including mimetics may be used in combination with aspirin. Aspirin and mimetics and compositions including mimetics may be administered together or separately, and if administered separately, aspirin may be administered by different routes of administration from administration of mimetics and compositions including mimetics, and may be administered on a different schedule and at different times than with administration of mimetics and compositions including mimetics.

In another aspect, natriuretic peptide mimetics and compositions including mimetics may be used in combination with positive inotropes, a class of drugs increasing myocardial contractility and used to support cardiac function. Such drugs may be administered together or separately, and if administered separately, may be administered by different routes of administration from administration of mimetics and compositions including mimetics, and may be administered on a different schedule and at different times than with administration of mimetics and compositions including mimetics. Such positive inotrope include, by way of example only and not limitation, berberine; calcium; calcium sensitisers such as levosimendan; cardiac myosin activators such as omecamtiv mecarbil; catecholamines such as dopamine, dobutamine, dopexamine, epinephrine (adrenaline), isoprenaline (isoproterenol) and norepinephrine (noradrenaline); digoxin; digitalis; eicosanoids such as prostaglandins; phosphodiesterase inhibitors such as enoximone, milrinone, amrinone and theophylline; glucagon; and insulin.

In another aspect, natriuretic peptide mimetics and compositions including mimetics may be used in combination with vasopressin receptor antagonists. Such drugs may be administered together or separately, and if administered separately, may be administered by different routes of administration from administration of mimetics and compositions including mimetics, and may be administered on a different schedule and at different times than with administration of mimetics and compositions including mimetics. Such vasopressin receptor antagonists include, by way of example only and not limitation, tolvaptan, conivaptan, relcovaptan, nelivaptan, lixivaptan, mozavaptan and satavaptan.

### Synthetic Methods of Amino Acid Surrogates

The following examples of methods of synthesis of amino acid surrogates utilized in the invention for making certain natriuretic peptide mimetics, including the mimetic of formula IV, are intended to be exemplary, and it is to be understood that variations thereon may be undertaken by one of skill in the art, and such variations are intended to be included herein.

Method A:

(2-Fmoc-amino-3-R′—O-propylamino)-2-substituted acetic acid methyl esters (10) were prepared by reductive amination of Fmoc O-protected serinal (9) with α-amino esters (2), using either sodium cyanoborohydride or sodium triacetoxyborohydride as the reducing agent. The Fmoc O-protected serinal (9) required for the reductive amination was prepared according to method D, either by reduction of the ester (12) by di-isobutylaluminun hydride, by oxidation of Fmoc O-protected serinol (13) with Dess-Martin periodinane, or by reduction of the Fmoc O-protected serine Weinreb amide (14) with lithium aluminum hydride. The preferred method for the preparation of Fmoc O-protected serinals (9) was the reduction of the Weinreb amide analog. (2-Fmoc-amino-3-R′—O-propylamino)-2-substituted acetic acid methyl esters (10) were then N and O deprotected, cyclized, and Fmoc protected to give 3-substituted 6-hydroxymethyl-piperazin-2-ones (6), which were then oxidized to the final product as described in method A.

The protecting group (R) on the hydroxyl group of Fmoc-O-protected serinal (9) used in the synthesis depends on the nature of the side chain R of the amino ester. When R contained no functional groups, the side chain of Fmoc serine was protected as the ^{t}Bu ether. When R contained functional groups, the side chain of Fmoc serine was protected as the trityl ether.

### Method A

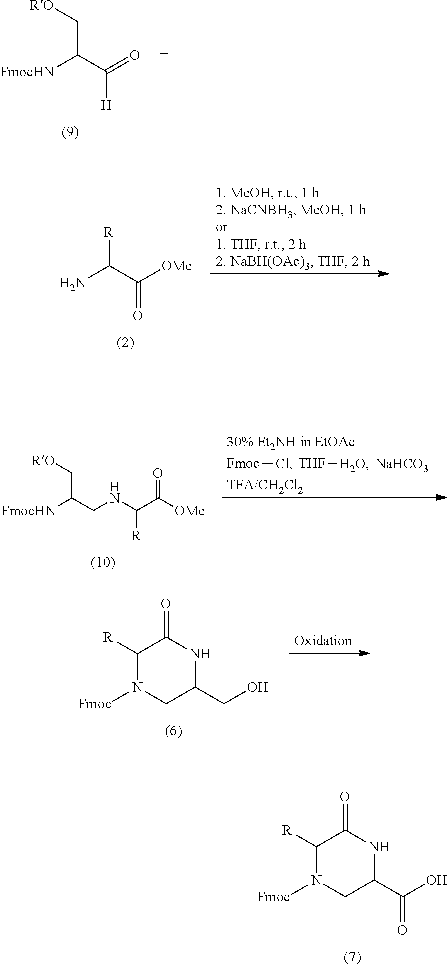

### Method B

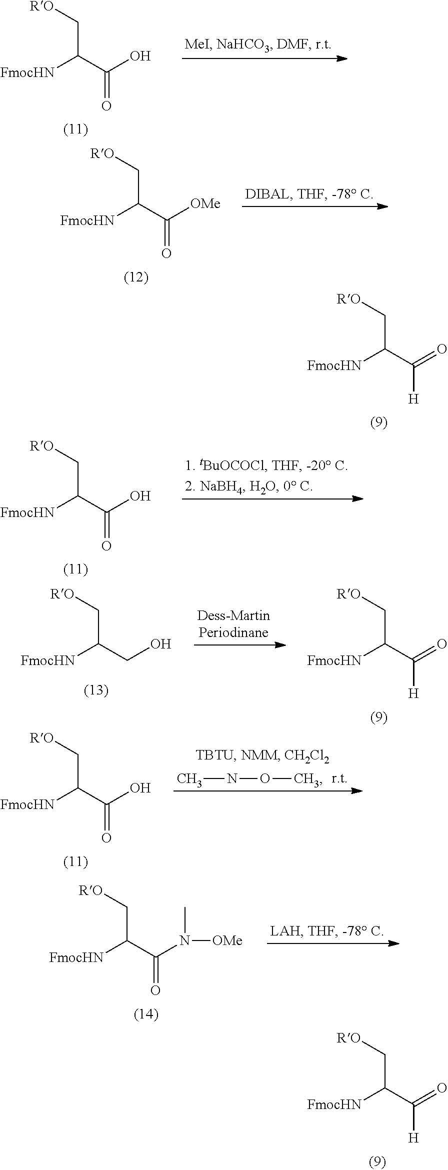

Method B:

Synthesis of various Fmoc-O-protected serinals (9). Synthesis of Fmoc-O—R′ serine methyl ester (12): A slight suspension of 80 mmol of Fmoc O—R′ serine (11), 10.0 g (120 mmol) of solid sodium bicarbonate, and 10.0 mL (160 mmol) of iodomethane in 80 mL of dry dimethylformamide, kept under nitrogen, was stirred at room temperature overnight. The reaction mixture was then poured over 500 mL of water, and the solid filtered. The solid was redissolved in 800 mL of ethyl acetate, and washed with 1×200 mL of water, dried over magnesium sulfate, and concentrated. No purification was required.

| R′ | Analytical Data for Compounds (12) |
|---|---|
| ^{t}Bu | ¹H NMR δ (CDCl₃): 1.14 (s, 9H, ^{t}Bu), 3.57-3.70 (m, 1H, CH₂—O), 3.75 (s, 3H, O—CH₃), |
| | 3.79-3.83 (m, 1H, CH₂—O), 4.01-4.50 (a series of multiples, 4H), 5.64-5.68 (d, 1H, |
| | NH), 7.28-7.78 (8H, fulvene), yield = 93% t_{R} = 7.8 min. |
| Trt | ¹H NMR δ (CDCl₃): 3.42-3.48 (m, 1H, CH₂—O), 3.59-3.66 (m, 1H, CH₂—O), 3.81 (s, 3H, |
| | CH₃—O), 4.10-4.18 (m, 1H, CH), 4.36-4.42 (m, 2H, CH₂—O), 4.50-4.57 (m, 1H, CH—N), |
| | 5.73-5.78 (d, 1H, NH), 7.22-7.82 (8H, fulvene), yield = quant., t_{R} = 9.04 min. |

Synthesis of Fmoc-O—R′ serinol (13): To a solution of 10.0 mmol of Fmoc O—R′ serine (11) in 50 mL of dry tetrahydrofuran, kept at −20° C. under nitrogen, was added 1.77 mL (12.7 mmol) of triethyl amine, followed by the slow addition of 1.57 mL (12.0 mmol) of isobutylchloroformate. The mixture was stirred for 30 minutes, and then poured slowly over an ice-cold solution of 3.77 g (99.6 mmol) of sodium borohydride in 10 mL of water, keeping the temperature below 5° C. The reaction was stirred at 0° C. for 15 minutes, and then quenched with 1N hydrochloric acid solution. The reaction mixture was diluted with 100 mL of ethyl acetate, and the layers separated. The organic layer was washed with 2×25 mL of 1N hydrochloric acid solution, 2×25 mL of water, dried over magnesium sulfate and concentrated. The compounds were purified by silica gel column chromatography.

| R′ | Analytical Data for Compounds (13) |
|---|---|
| ^{t}Bu | ¹H NMR δ (CDCl₃): 1.14 (s, 9H, ^{t}Bu), 2.90-2.95 (d, 1/2H, CH₂—O), 3.63 (d, 2H, CH₂—O), |
| | 3.65-3.93 (m, 3H, CH₂—O), 4.20-4.35 (t, 1H, CH), 4.35-4.45 (d, 2H, CH₂), 5.50- |
| | 5.57 (d, 1H, NH), 7.26-7.8 (8H, fulvene), yield = 85%, t_{R} = 6.42 min. |
| Trt | ¹H NMR δ (CDCl₃): 3.24-3.32 (br. d, 1H, CH₂—O), 3.30-3.45 (br. m, 1H, CH₂—O), 3.60- |
| | 3.987 (br. m, 3H, CH₂—O, and CH—N), 4.13-4.22 (br. m, 1H, CH), 4.32-4.40 (br. d, 2H, |
| | CH₂), 5.24-5.32 (br. d, 1H, NH), 7.16-7.76 (23H, fulvene, and Trt), yield = 92%, t_{R} = |
| | 8.39 min. |

Synthesis of Fmoc-O—R′ serine Weinreb amide (14): A suspension of 20.2 mmol of Fmoc O—R′ serine (11), 6.98 g (21.6 mmol) of 2-(1H-benzotriazol-1-yl)-1,1,3,3-tetramethyluronium tetrafluoroborate (TBTU), and 2.5 mL (22.7 mmol) of N-methyl-morpholine in 80 mL of dry dichloromethane was stirred at room temperature under nitrogen for 20 minutes, and then 3.02 g (31 mmol) of N,O-di-methyl-hydroxylamine hydrochloride and 3.3 mL (30 mmol) of N-methyl-morpholine were added, and the suspension stirred at room temperature overnight. The solution formed was then concentrated to dryness, repartitioned between 200 mL of ethyl acetate and 100 mL of water, washed with 2×40 mL of 1N hydrochloric acid solution and then 2×40 mL of saturated sodium bicarbonate solution, dried over magnesium sulfate, and concentrated. No purification was required.

| R′ | Analytical Data for Compounds (14) |
|---|---|
| ^{t}Bu | ¹H NMR δ (CDCl₃): 1.45 (s, 9H, ^{t}Bu), 3.30 (s, 3H, CH₃—N), 3.55-3.7 (m, 2H, CH₂—O), |
| | 3.76 (S, 3H, CH₃—O), 4.19-4.26 (m, 1H, CH), 4.30-4.38 (m, 2H, CH₂—O), 4.82-4.91 |
| | (broad m, 1H, CHN), 5.68-5.75 (d, 1H, NH), 7.2-7.8 (8H, fulvene), yield = quant., t_{R} = |
| | 6.59 min. |
| Trt | ¹H NMR δ (CDCl₃): 3.24 (s, 3H, CH₃N), 3.34-3.46 (m 2H, CH₂O), 3.62 (s, 3H, CH₃O), |
| | 4.15-4.37 (two m, CH₂, CH), 4.86-4.98 (m 1H, CHN), 5.80-5.86 (d, 1H, NH), 7.18-7.8 |
| | (a series of m, 23H, Trt and fulvene), yield = quant., t_{R} = 8.0 min. |

Synthesis of Fmoc-O—R′ serinal (9) from ester (12): To a solution of 3.5 mmol of (12) in 5 mL of tetrahydrofuran, kept at −78° C. under nitrogen, was added slowly 10 mL of 1N diisobutyl aluminum hydride (DIBAL) solution, stirred for 15 minutes, and quenched by the slow addition of a saturated solution of sodium and potassium tartrate. The reaction was allowed to warm up to room temperature, diluted with 50 mL of ethyl acetate, and 50 mL of a saturated solution of sodium and potassium tartrate was added. The layers were separated, and the aqueous layer re-extracted with 1×50 mL of ethyl acetate. The organic layers were combined, dried over magnesium sulfate, and concentrated. Compounds (9) were used without further purification in the next step.

| R′ | Analytical Data for Compounds (9) |
|---|---|
| ^{t}Bu | ¹H NMR δ (CDCl₃): 1.16 (s, 9H, ^{t}Bu), 3.59-3.66 (dd, 1H, CH₂O), 3.90-3.98 (dd, 1H, |
| | CH₂O), 4.20-4.27 (t, 1H, CH), 4.32-4.45 (two m, 3H, CHN, and CH₂O), 5.64-5.74 (br. |
| | d, 1H, NH), 7.28-7.35 (m, 2H, fulvene), 7.36-7.44 (m, 2H, fulvene), 7.58-7.65 (d, 2H, |
| | fulvene), 7.73-7.78 (d, 2H, fulvene), 9.62 (s, 1H, CHO). |
| Trt | ¹H NMR δ (CDCl₃): 3.53-3.61 (dd, 1H, CH₂O), 3.66-3.75 (dd, 1H, CH₂O), 4.33-4.47 |
| | (two m, 4H, CHN, CH, and CH₂), 5.66-5.75 (d, 1H, NH), 7.20-7.81 (a series of m, 23H, |
| | Trt, and fulvene), 9.6 (s, 1H, CHO). |

Synthesis of Fmoc-O—R′ serinal (9) from alcohol (13): To a solution of 80 mmol of Fmoc-O—R′ serinol (13) in 200 mL of dry dichloromethane, kept at room temperature under nitrogen, was added 88 mmol of Dess-Martin periodinane, and the reaction was stirred for 2.5 hours and quenched by addition of 400 mL of 10% aqueous sodium thiosulfate solution. The layers were separated, and the organic layer concentrated, diluted with 300 mL of ethyl ether, and washed three times with a saturated aqueous bicarbonate solution containing 10% sodium thiosulfate, dried over magnesium sulfate, and concentrated.

Synthesis of Fmoc-O—R′ serinal (9) from Weinreb amide (14): To a solution of 8.8 g (20.2 mmol) of crude Fmoc-O—R′ serine Weinreb amide intermediate (14) in 60 mL of dry tetrahydrofuran, cooled to −78° C. under nitrogen, was added 30 mL of 1N lithium aluminum hydride solution in tetrahydrofuran. The solution was stirred for 15 minutes and then quenched by the slow addition of 30 mL of a 1.4N solution of potassium hydrogen sulfate. After warming up to room temperature, the solid was filtered and the filtrate concentrated to dryness. The residue was repartitioned between 50 mL of ethyl acetate and 25 mL of 1N hydrochloric acid solution. The layers separated, and the organic layer was dried over magnesium sulfate, filtered, and concentrated.

Synthesis of (2-Fmoc-amino-3-R′—O-propylamino)-2-substituted acetic acid methyl ester (10): compounds (10) were prepared by reductive amination using sodium cyanoborohydride or sodium triacetoxyborohydride as the reducing agent.

Sodium cyanoborohydride method: To a solution of 8.5 mmol of (2) hydrochloride salt in 20 mL of methanol, kept at room temperature under nitrogen, was added 2.3 mmol of solid potassium hydroxide, and the mixture stirred for 25 minutes. A solution of Fmoc-O—R′ serinal (9) in 10 mL of methanol was added to the above suspension, and the reaction mixture was stirred for 1 hour. A solution of 8.5 mL of 1N sodium cyanoborohydride in tetrahydrofuran was added slowly, and the reaction stirred for another 1 hour, filtered, and concentrated. The residue was partitioned between ethyl acetate and water, and the organic layer washed with 1×20 mL of saturated sodium bicarbonate, dried over sodium sulfate, and concentrated.

Sodium triacetoxyborohydride method: A suspension of 21 mmol of (2) hydrochloride salt, and 2.9 mL (21 mmol) of triethyl amine in 50 mL of dry tetrahydrofuran, was stirred at room temperature for 45 min, and then a solution of ˜20 mmol crude Fmoc-(O—R′)-serinal (9) in 30 mL of tetrahydrofuran was added, followed by 1.7 g of 4 A powdered molecular sieves, and the suspension was stirred for an additional 2 h. 6.4 g (30 mmol) of solid sodium triacetoxyborohydride was added, and the suspension stirred at room temperature overnight. The suspension was diluted with methanol, the molecular sieves filtered, and the filtrate concentrated. The residue was partitioned between 100 mL of ethyl acetate and 50 mL of water. The organic layer was dried over sodium sulfate, filtered, and concentrated.

Compounds (10) were purified by silica gel column chromatography.

| R′ | R | Analytical Data for Compounds (10) |
|---|---|---|
| ^{t}Bu | 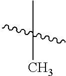 | ¹H NMR δ (CDCl₃): 1.17 (s, 9H, ^{t}Bu), 1.26-1.32 (d, 3H, CH₃), 2.68-2.80 (br. m, 2H, CH₂N), 3.32-3.56 (two br. m, 2H, CH₂O), 3.72 (s, 3H, CH₃O), 3.66-3.82 (m, 1H, CHN), |
| | | 4.18-4.28 (t, 1H, CH), |
| | | 4.30-4.46 (d, 2H, CH₂), |
| | | 5.34-5.44 (br. d, 1H, NH), |
| | | 7.25-7.44 (two m, 4H, fulvene), |
| | | 7.59-7.64 (d, 2H, fulvene), |
| | | 7.74-7.79 (d, 2H, fulvene), |
| | | yield = 57%, t_{R} = 4.93 min, |
| | | (M⁺ + 1) = 455.67. |
| ^{t}Bu | 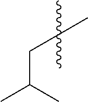 [SMILES: *CC(C)C] | ¹H NMR δ (CDCl₃): 0.88- 0.98 (br. t, 6H CH₃), 1.21 (s 9H, ^{t}Bu), 1.26-1.34 (m, 2H, CH₂), 1.44-1.54 (m, 1H, CH), 2.58-2.86 (two m, 1H, CH₂N), 3.25-3.35 (m, 1H, CH₂N), 3.37-3.58 (two m, 2H, CH₂O), |
| | | 3.72-3.80 (br. m, 1H, CHN), |
| | | 4.14-4.31 (m, 1H, CH), |
| | | 4.32-4.45 (br. d, 2H, CH₂O), |
| | | 5.34-5.44 (br. d, 1H, NH), |
| | | 7.30-7.84 (a series of m, 8H, fulvene), |
| | | yield = 50%, t_{R} = 5.66 min, |
| | | (M⁺ + 1) = 511.67. |
| ^{t}Bu | 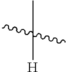 [SMILES: *[H]] | ¹H NMR δ (CDCl₃): 1.17 (s, 9H, ^{t}Bu), 2.68-2.78 (m, 1H, CH₂N), 2.82-2.92 (m, 1H, CH₂N), 3.35- 3.55 (m, 4H, CH₂N, and CH₂O), 3.73 (s, 3H, CH₃O), |
| | | 3.75-3.85 (m, 1H, CHN), |
| | | 4.20-4.28 (m, 1H, CH), |
| | | 4.32-4.48 (m, 2H, CH₂), |
| | | 5.40-5.50 (d, 1H, NH), 7.28- |
| | | 7.8 (a series of m,8H, fulvene), |
| | | yield = 44%, t_{R} = 5.02 min, |
| | | (M⁺ + 1) = 441.50. |
| ^{t}Bu | 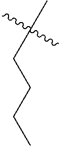 [SMILES: *CCCC] | ¹H NMR δ (CDCl₃): 0.84- 0.92 (br. t, 3H, CH₃), 1.17 (s, 9H, ^{t}Bu), 1.28-1.35 (m, 4H, CH₂), 1.48-1.84 (two m, 2H, CH₂), 2.62-2.82 (m, 2H, CH₂N), 3.20-3.33 (m, 1H, CHN), 3.35-3.54 (two m, 2H, CH₂O), 3.72 (s, 3H, CH₃O), |
| | | 3.64-3.80 (m, 1H, CHN), |
| | | 4.20-4.28 (t, 1H, CH), |
| | | 4.32-4.42 (m, 2H, CH₂O), |
| | | 5.34-5.44 (br. d, 1H, NH), |
| | | 7.25-7.79 (a series of m, 8H, fulvene), |
| | | yield = 65%, t_{R} = 5.85 min, |
| | | (M⁺ + 1) = 441.27. |
| Trt | 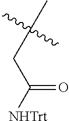 | ¹H NMR δ (CDCl₃): 2.36- 2.63 (br. m, 2H, CH₂CO), 2.65-2.90 (br. m, 2H, CH₂N), 3.05-3.20 (br. m, 2H, CH₂O), 3.50-3.64 (br. m, 1H, CHN), 3.68 & 3.69 (two s, 3H, CH₃O), 3.82-3.94 (br. m, 1H, CHN), 4.12-4.21 (br. m, 1H, CH), |
| | | 4.24-4.43 (br. m, 2H, CH₂O), |
| | | 4.90-4.98 (br. d, 1H, NH), |
| | | 7.15-7.80 (a series of m, 23H, |
| | | fulvene and Trt), |
| | | yield = 39%, t_{R} = 8.13 min, |
| | | (M⁺ + 1) = 926.99. |
| Trt | 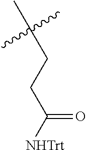 | ¹H NMR δ (CDCl₃): 1.68- 1.82 (m, 1H, CH₂), 1.85-1.99 (m, 1H, CH₂), 2.12-2.37 (m, 2H, CH₂CO), 2.58-2.96 (a series of four m, 2H, CH₂N), 3.08-3.28 (br. m, 2H, CH₂O), 3.66 & 3.67 (two s, 3H, CH₃O), 3.76-3.89 (br. m, 1H, CHN), 4.15-4.24 (br. m, 1H, CH), 4.28-4.41 (br. d, 2H, CH₂O), |
| | | 5.10-5.22 (br. d, 1/2H, NH), |
| | | 5.28-5.35 (br. d, 1/2H, NH), |
| | | 7.15-7.80 (a series of m, |
| | | 23H, fulvene, and Trt), |
| | | yield = 43%, t_{R} = 8.10 min, |
| | | (M⁺ + 1) = 940.97. |
| Trt | 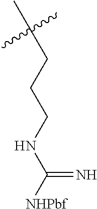 | ¹H NMR δ (CDCl₃): 1.43 (s, 6H, CH₃), 1.46-1.56 (m, 4H, CH₂), 2.06 (s, 3H, CH₃), 2.50 (s, 3H, CH₃), 2.57 (s, 3H, CH₃), 2.75-2.80 (m, 1H, CH₂N), 2.91 (s, 2H, CH₂), 3.12-3.32 (three br. m, 4H, CH₂N), 3.68 (s, 3H, CH₃O), 4.13-4.21 (t, 1H, CH), 4.28-4.38 (d, 2H, CH₂), 5.12-5.23 (br. d, 1H, NH), 5.80-6.12 (two br. m, 2H, NH), 7.18-7.80 (a series of |
| | | m, 23H, fulvene, and Trt), yield = 68%, |
| | | t_{R} = 7.52 min, (M⁺ + 1) = 997.91. |
| Trt | 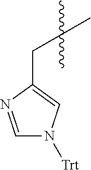 | ¹H NMR δ (CDCl₃): 2.75- 2.98 (two m, 2H, CH₂N), 3.06-3.18 (m, 1H, CH₂N), 3.22-3.33 (m, 1H, CH₂N), 3.57 & 3.60 (two s, 3H, CH₃O), 3.80-3.92 (m, 1H, CHN), 4.00-4.08 (m, 1H, CH), 4.18-4.30 (br. d, 2H, CH₂), 7.00-7.80 (a series of m, 25H, fulvene, Trt, and Imidazole), |
| | | yield = 57%, t_{R} = 7.59 min, |
| | | (M⁺ + 1) = 949.79. |
| Trt | 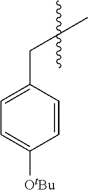 [SMILES: *Cc1ccc(OC(C)(C)C)cc1] | ¹H NMR δ (CDCl₃): 1.26 & 1.27 (two s, 9H, ^{t}Bu), 2.50-2.61 (dd, 1H, CH₂—Ph), 2.76-2.86 (m, 2H, CH₂—Ph, and CH₂N), 2.92-3.20 (m, 1H, CH₂N), 2.92-3.20 (m, 2H, CH₂O), 3.32-3.46 (m, 1H, CH₂O), 3.59 (s, 3H, CH₂O), 3.79-3.88 (m, 1H, CHN), 4.18-4.28 (m, 1H, CH), 4.30-4.37 (br. d, 2H, CH₂O), 5.18-5.26 (br. d, 1H, NH), |
| | | 6.80-6.88 (d, 2H, Ph), |
| | | 6.96-7.02 (d, 2H, Ph), |
| | | 7.18-7.80 (a series of m, |
| | | 23H, fulvene, and Trt), |
| | | yield = 23%. |
| Trt | 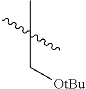 [SMILES: *COC(C)(C)C] | ¹H NMR δ (CDCl₃): 1.11 (s, 9H, ^{t}Bu), 2.54-2.74 (two m, 2H, CH₂N), 3.02-3.58 (six m, 6H, CH₂O, CH₂N, and CHN), 3.70 (s, 3H, CH₃O), 3.83-3.93 (m, 1H, CHN), |
| | | 4.15-4.29 (m 1H, CH), |
| | | 4.34-4.37 (d, 2H, CH₂), |
| | | 5.46-5.53 (br. d, 1H, NH), |
| | | 7.18-7.79 (a series of m, |
| | | 23H, fulvene, |
| | | and Trt), yield = 45%, |
| | | (M⁺ + 1) = 713.42. |
| ^{t}Trt | 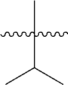 [SMILES: *C(C)C] | ¹H NMR δ (CDCl₃): 0.80- 0.92 (m, 7H, CH₃), 1.75-1.90 (br. m, 1H, CH), 2.6-4.36 (a series of m, 9H, CH₂O, CH₂N, CHN), 3.68 (s, 3H, CH₃O), |
| | | 5.5 (d, 0.5H, CH), 7.23-7.77 (m, |
| | | 24H, fulvene and Trt), |
| | | yield = 72% (3 steps), t_{R} = 6.86 min, |
| | | (M⁺ + 1) = 669.10. |

Synthesis of 4-Fmoc-6-hydroxymethyl-3-substituted-piperazin-2-one (6): For the preparation of compounds (6) three steps were required: (a) Fmoc deprotection with concomitant cyclization, (b) Fmoc protection, and (c) hydroxyl group deprotection.

Fmoc group removal and cyclization: A solution of 10 mmol of cyclic compound in 30 mL of 30% diethyl amine in ethyl acetate solution was stirred at room temperature overnight, and then concentrated to dryness.

(a) Fmoc protection: To a biphasic solution of 10 mmol of compound in 20 mL of tetrahydrofuran and 10 mL of water, was added 2.52 g (30 mmol) of solid sodium bicarbonate, followed by 3.36 g (13 mmol) of Fmoc-Cl. The mixture was stirred for 3 hours, diluted with ethyl acetate, the layers separated, and the organic layer washed with water, dried over magnesium sulfate, and concentrated.

(b) Hydroxyl group deprotection: For compounds containing a ^{t}Bu ether protecting group: The compounds were deprotected with a solution of 90% trifluoroacetic acid in dichloromethane for 1-2 hours, and then concentrated to dryness. The residue was dissolved in ethyl acetate and washed with a saturated solution of sodium bicarbonate, dried over magnesium sulfate, and then concentrated. For compounds containing a Trt ether protecting group: the compounds were deprotected by adding a solution of 1-10% trifluoroacetic acid in dichloromethane containing 2-10% tri-isopropyl silane. The reaction was instantaneous. The solution was then neutralized by pouring it into a saturated solution of sodium bicarbonate. The layers were separated, dried over sodium sulfate, and concentrated.

Compounds (6) were purified by silica gel column chromatography.

| R | Analytical Data for Compounds (6) |
|---|---|
| 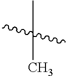 | ¹H NMR δ (CDCl₃): 1.17- 1.35 (br. m, 3H, CH₃), 2.64-2.82 (t, 1H, CH₂N), 3.2-3.8 (two br. m, 3H, CH₂O, CH₂N), 4.18-4.44 (br. t, 1H, CH), |
| | 4.64-4.90 (br. d, 2H, CH₂O), |
| | 6.70-6.86 (br. s, 1H, NH), |
| | 7.22-7.82 (a series of m, 8H, fulvene), |
| | yield = 72%, t_{R} = 4.64 min, |
| | (M⁺ + 1) = 367.32. |
| 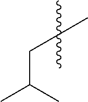 [SMILES: *CC(C)C] | ¹H NMR δ (CDCl₃): 0.64- 1.02 (m, 6H, CH₃), 1.45-1.63 (m, 3H, CH₂, and CH), 2.65-2.84 (m, 1H, CH₂N), 2.89-3.76 (a series of br. m, 5H, CH₂O, and CHN), |
| | 4.17-4.28 (br. m, 1H, CH), |
| | 4.48-4.82 (three br. m, CH₂O, |
| | NH, and OH), |
| | 6.95-7.82 (a series of br. m, |
| | 8H, fulvene), |
| | yield = 51%, t_{R} = 5.43 min, |
| | (M⁺ + 1) = 409.08. |
| 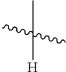 [SMILES: *[H]] | ¹H NMR δ (CDCl₃): 3.17- 3.78 (a series of br. m, 5H, CH₂O, CH₂N, and CHN), 4.21-4.27 (t, 1H, CH), 4.42-4.68 (br. peak, 2H, CH₂O), |
| | 6.62 (br. s, 1H, NH), |
| | 7.28-7.81 (a series of m, |
| | 8H, fulvene), |
| | yield = 67%, t_{R} = 4.50 min, |
| | (M⁺ + 1) = 353.45. |
| 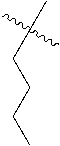 [SMILES: *CCCC] | ¹H NMR δ (CDCl₃): 0.72- 0.90 (br. peak, 3H, CH₃), 1.0-1.40 (br. peak, 4H, CH₂), 1.48-1.90 (three br. peaks, 2H, CH₂), 2.68-2.80 (t, 1H, CH₂N), 3.10-3.70 (four br. peaks, 4H, CH₂O, CHN, and CH₂N), 4.15-4.25 (br. peak, 1H, CH), 4.54-4.62 (br. d, 2H, CH₂O), |
| | 7.25-7.80 (a series of m, 8H, fulvene), |
| | yield = 72%, t_{R} = 5.77 min, |
| | (M⁺ + 1) = 408.95. |
| 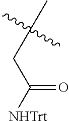 | ¹H NMR δ (CDCl₃): 2.50- 3.38 (four overlapping br. m, 7H, CH₂—CO, CH₂N, CH₂O, and CHN), 3.42-3.64 (m, 1/2H, CHN), 3.70-3.88 (m, 1/2H, CHN), 4.16-4.23 (br. d, 1H, CH), 4.48-4.68 (br. m, 2H, CH₂O), |
| | 4.94-5.05 (br. d, 1H, NH), |
| | 6.95-7.80 (a series of m, |
| | 23H, fulvene and Trt), |
| | yield = 83%, t_{R} = 7.04 min, |
| | (M⁺ + 1) = 652.61. |
| 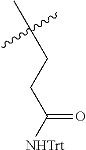 | ¹H NMR δ (CDCl₃): 1.67- 1.78 (br. m, 1H, CH₂), 1.81-2.0 (br. m, 1H, CH₂), 2.10-2.43 (br. m, 2H, CH₂—CO), 2.58-2.81 (br. m, 2H, CH₂N), 3.02-3.66 (a series of br. m, 4H, CH₂O and CHN), 4.17-4.23 (br. m, 1H, CH), 4.40-4.80 (br. m, 2H, CH₂O), 7.15-7.80 (a series of m, |
| | 23H, fulvene, and Trt), |
| | yield = 80%, t_{R} = 7.04 min, |
| | (M⁺ + 1) = 666.66. |
| 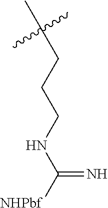 | ¹H NMR δ (CDCl₃): 1.43 (s, 6H, CH₃), 1.50-1.60 (br. m, 4H, CH₂), 2.10 (s, 3H, CH₃), 2.48 (s, 3H, CH₃), 2.55 (s, 3H, CH₃), 2.92 (s, 2H, CH₂), 3.08-3.47 (two m, 3H, CH₂O, and CH₂N), 3.57-3.97 (a series of m, 3H, CH₂O, and CHN), 4.15-4.25 (br. m, 1H, CH), 4.44-4.74 (br. m, 2H, CH₂), 7.20-7.80 (a series of br. m, 8H, fulvene), yield = 91%, t_{R} = 6.05 min, (M⁺ + 1) = 704.71. |
| 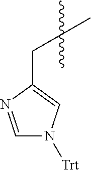 | ¹H NMR δ (CDCl₃): 2.14-2.56 (two m, 2H, CH₂—Im), 2.90-3.90 (a series of m, 4H, CH₂N, and CH₂O), 4.0-4.74 (a series of m, 4H, CHN, CH, CH₂), 7.0-7.80 (a series of multiples, 25H, fulvene, Im, and Trt), yield = 64%, t_{R} = 5.27 min, (M⁺ + 1) = 675.08. |
| 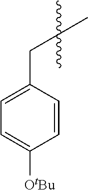 [SMILES: *Cc1ccc(OC(C)(C)C)cc1] | ¹H NMR δ (CDCl₃): 1.29 (s, 9H, ^{t}Bu) 2.47- 2.74 (a series of m, 2H, CH₂Ph), 2.90-3.04 (m, 1H, CH₂Ph), 3.06-3.45 (three m, 6H, CH₂O, and CH₂N), 3.89-4.29 (three m, 2H, CH, and CHN), 4.32-4.42 (m, 1H, CHN), 4.56-4.66 (m, 2H, CH₂), 6.81-7.80 (a series of m, |
| | 12H, fulvene, and Ph), |
| | yield = 71%, (M⁺ + 1) = 515.81. |
| 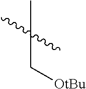 [SMILES: *COC(C)(C)C] | ¹H NMR δ (CDCl₃): 1.00 & 1.10 (two s, 9H, ^{t}Bu), 3.0-3.74 (four br. m, 7H, CH₂O, CH₂N, and CHN), 3.86-4.26 (a series of m, 2H, CHN, and CH), |
| | 4.42-4.68 (br. d, 2H, CH₂), |
| | 7.26-7.80 (a series of br. m, |
| | 8H, fulvene), |
| | yield = 55%, (M⁺ + 1) = 439.08. |

Synthesis of 4-Fmoc-5-substituted-6-oxo-piperazine-2-carboxylic acid (7): Compounds (7) were prepared as described in method A. Compounds (7) were purified by silica gel column chromatography.

| R | Analytical Data for Compounds (7) |
|---|---|
| 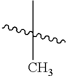 | ¹H NMR δ (CDCl₃): 1.08- 1.20 (br. peak, 1.5H, CH₃), 1.30-1.38 (br. peak, 1.5H, CH₃), 2.86-3.07 (br. m, 1H, CH₂N), 3.83-3.97 (br. m, 1H, CH₂N), |
| | 4.18-4.37 (a series of br. peaks, |
| | 2H, CH and CHN), |
| | 4.40-4.74 (two br. peaks, |
| | 3H, CH₂O, and CHN), |
| | 7.28-7.82 (a series of m, 8H, fulvene), |
| | 8.92-9.10 (br. s, 1H, CO₂H), |
| | yield = 51 %, t_{R} = 4.80 min, |
| | (M⁺ + 1) = 381.57. |
| 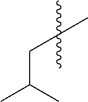 [SMILES: *CC(C)C] | ¹H NMR δ (CDCl₃): 0.40- 1.60 (a series of br. peaks, 9H, CH, CH₂ and CH₃), 2.81-3.09 (br. peak, 1H, CH₂N), 3.68-3.80 (br. peak, 2H, CHN), 3.96-4.32 (br. peaks, 2H, CH, and CNH), 4.48-4.68 (br. peak, CH₂O), |
| | 7.26-7.84 (a series of m, 8H, fulvene), |
| | yield = 50%, t_{R} = 5.57 min, |
| | (M⁺ + 1) = 423.15. |
| 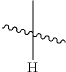 [SMILES: *[H]] | ¹H NMR δ (CDCl₃): 3.77- 3.99 (m, 1H, CHN), 3.90-4.35 (a series of m, 5H, CH₂N, CH), 4.44-4.57 (d, 2H, CH₂), |
| | 7.3-7.82 (a series of m, 8H, fulvene), |
| | yield = 48%, t_{R} = 4.58 min, |
| | (M⁺ + 1) = 367.30. |
| 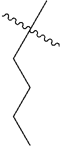 [SMILES: *CCCC] | ¹H NMR δ (CDCl₃): 0.69- 1.90 (a series of br. peaks, CH₂, and CH₃), 2.85-3.05 (br. peak, 2H, CH₂N), 3.65-3.95 (two br. peaks, 1H, CHN), 4.00-4.40 (two br. peaks, CH₂N, and CH), 4.41-4.74 (br. peak, 3H, CH₂O, and CHN), 7.20-7.80 (a series of br. m, 8H, fulvene), yield = 70%, t_{R} = 5.93 min, |
| | (M⁺ + 1) = 423.42. |
| 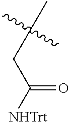 | ¹H NMR δ (CDCl₃): 2.51- 3.06 (a series of m, 2H, CH₂—CO), 3.85-4.86 (a series of m, 7H, CH₂N, CHN, CH, and CH₂O), 7.0-7.78 (a series of br. m, 23H, fulvene and Trt), yield = 30%, t_{R} = 7.04 min, (M⁺ + 1) = 666.79. |
| 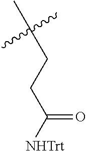 | ¹H NMR δ (CDCl₃): 1.74- 2.46 (a series of br. m, 4H, CH₂—CO, and CH₂), 3.78-4.06 (two m, 2H, CH₂N), 4.16-4.68 (a series of br. m, 5H, CHN, CH, and CH₂O), 7.14-7.82 (a series of br. m, 23H, fulvene, and Trt) yield = 47%, t_{R} = 7.11 min, (M⁺ + 1) = 680.33. |
| 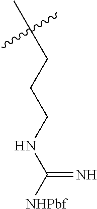 | ¹H NMR δ (CDCl₃): 1.08- 1.60 (a series of br. peaks, 8H, CH₂, and CH₃), 2.12 (s, 3H, CH₃), 2.48 (s, 3H, CH₃), 2.57 (s, 3H, CH₃), 2.92 (s, 2H, CH₃), 3.10-3.25 (br. m, 2H, CH₂N), 3.82-4.28 (a series of br. m, 4H, CH₂N, CHN, CH), 4.40-4.70 (br. m, 3H, CHN, and CH₂O), 7.20-7.80 (a series of br. m, 8H, fulvene), yield = 42%, t_{R} = 6.15 min, (M⁺ + 1) = 718.69. |
| 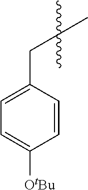 [SMILES: *Cc1ccc(OC(C)(C)C)cc1] | ¹H NMR δ (CDCl₃): 1.28 & 1.34 (two s, 9H, ^{t}Bu), 2.42-3.64 (a series of br. m, 5H, CH₂N, CHN, and CH₂Ph), 4.0-4.76 (a series of br. m, 4H, CHN, CH, and CH₂O), 6.60-6.96 (br. m, 4H, Ph), 7.20-7.80 (a series of br. m, 8H, fulvene), yield = 67%, (M⁺ + 1) = 529.17. |
| 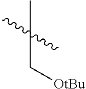 [SMILES: *COC(C)(C)C] | ¹H NMR δ (CDCl₃): 0.96- & 1.10 (two s, 9H, ^{t}Bu), 3.04-3.18 (br. m, 0.5H, CH₂N), 3.30-3.94 (four br. m, 3.5H, CH₂N, and CH₂O), 3.98-4.32 (br. m, 2H, CH, and CHN), |
| | 4.33-4.74 (two br. m, 3H, CHN, CH₂O), |
| | 7.28-7.80 (a series of m, 8H, fulvene), |
| | yield = 60%, (M⁺ + 1) = 453.37. |

Method C: Diphenylmethyl 3-Fmoc-amino-4-(methoxycarbonyl-substituted-methylamino)-butyrates (41) were prepared by reductive amination of diphenylmethyl 3-Fmoc-amino-4-oxo-butyrate (40) with α-amino esters (2), using either sodium cyanoborohydride or sodium triacetoxyborohydride as the reducing agent. The diphenylmethyl 3-Fmoc-amino-4-oxo-butyrate (40) required for the reductive amination was prepared by lithium aluminum hydride reduction of the Weinreb amide derivative (39), which was formed from commercially available Fmoc-aspartic acid α-allyl ester derivative (38) by protection of the β-ester under Mitsunobu conditions. The allyl ester was removed using palladium (0) catalyst, followed by Weinreb amide formation using TBTU as the coupling agent. Diphenylmethyl 3-Fmoc-amino-4-(methoxycarbonyl-substituted-methylamino)-butyrate (41) was then Fmoc deprotected, cyclized, diphenylmethyl ester removed by hydrogenation, followed by Fmoc protection to give the final product (4-Fmoc-5-substituted-6-oxo-piperazin-2-yl)-acetic acid (37).

### Method J

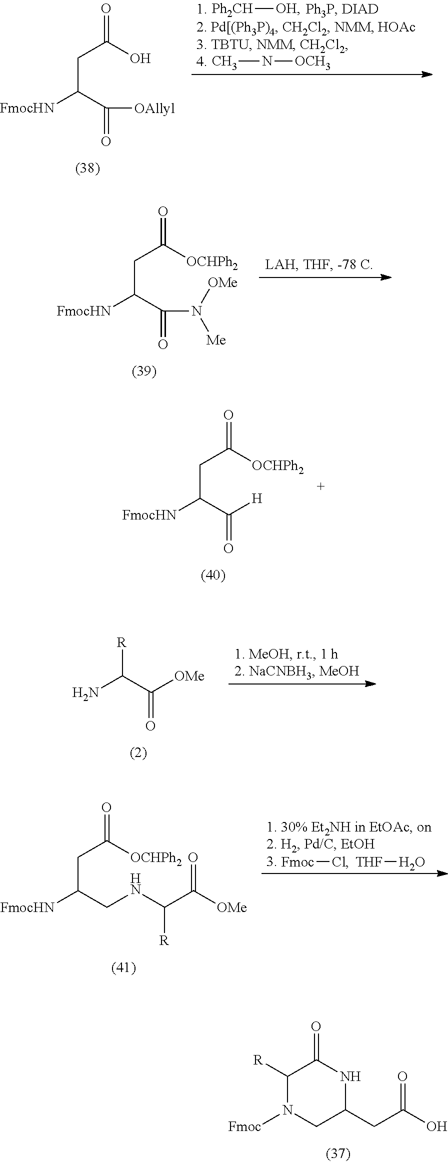

Synthesis of Fmoc-Asp-(OCHPh₂) Weinreb amide (39): To a solution of 5.1 g (13.0 mmol) of Fmoc-aspartic acid α-allyl ester (38) in 30 mL of dry tetrahydrofuran, containing 3.4 g (13 mmol) of triphenylphosphine, and 2.41 g (13.1 mmol) of diphenylmethanol, kept at 0° C. under nitrogen, was added slowly 2.6 mL (13.4 mmol) of diisopropyl azodicarboxylate. The ice bath was removed, and the reaction stirred at room temperature overnight, concentrated to dryness, and then purified by silica gel column chromatography. ¹H NMR 6 (CDCl₃) 2.96-3.06 (dd, 1H, CH₂CO), 3.15-3.26 (dd, 1H, CH₂CO), 4.18-4.76 (a series of m, 3H, CH, CH₂), 5.14-5.32 (m, 1H, CHN), 5.76-5.86 (m, 1H, CHO), 7.20-7.80 (a series of m, 18H, fulvene, and Ph); HPLC t_{R}=7.68 min, (M⁺+Na⁺)=583.90.

The product (9.8 mmol) was then dissolved in 40 mL of a dichloromethane:acetic acid:N-methyl morpholine solution at 37:2:1, containing 1.5 g (1.3 mmol) of tetrakis triphenylphosphine palladium (0), and the solution stirred at room temperature overnight, concentrated to dryness, and partitioned between 100 mL of ethyl acetate and 30 mL of water. The layers were separated, and the organic layer washed with 1×50 mL of water, dried over sodium sulfate, and concentrated. The residue was suspended in 20 mL of dry dichloromethane, and 1.65 mL (15 mmol) of N-methyl morpholine, and 4.07 g (12.7 mmol) of TBTU were added, and the suspension stirred at room temperature for 20 minutes, followed by the addition of 1.65 mL (15 mmol) of N-methyl morpholine, and 1.52 g (15.6 mmol) of N,O-dimethyl hydroxylamine hydrochloride salt. The suspension was stirred at room temperature for 2 hours, concentrated, partitioned between 100 mL of ethyl acetate and 50 mL of water. The organic layer was washed with 1×30 mL of water, 1×30 mL of saturated sodium bicarbonate solution, and 1×30 mL of 1N hydrochloric acid solution, dried over sodium sulfate, and concentrated. The product was purified by silica gel column chromatography. ¹H NMR 6 (CDCl₃) 2.76-2.88 (dd, 1H, CH₂CO), 2.89-3.00 (dd, 1H, CH₂CO), 3.16 (s, 3H, CH₃N), 3.70 (s, 3H, CH₃O), 4.14-4.22 (dd, 1H, CH), 4.28-4.40 (t, 2H, CH₂), 5.07-5.16 (dd, 1H, CHN), 5.69-5.76 (d, 1H, CHO), 7.24-7.8 (a series of m, 18H, fulvene, and Ph); HPLC t_{R}=7.08, (M⁺+Na⁺)=587.03.

Synthesis of Diphenylmethyl 3-Fmoc-amino-4-oxo-butyrate (40): Compound (40) is prepared using a procedure similar to the one described for compound (9).

Synthesis of Diphenylmethyl 3-Fmoc-amino-4-(methoxycarbonyl-substituted-methylamino)-butyrate (41): Compounds (41) were prepared using a procedure similar to the one described for compound (10), but using compound (40) as the aldehyde.

| R | Analytical Data for Compounds (41) |
|---|---|
| 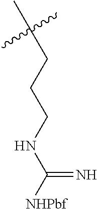 | ¹H NMR δ (CDCl₃) 1.2-1.7 (m, 4H, CH₂), 1.42 (s, 3H, CH₃Ph), 1.60 (s, 6H, CH₃—Ph), 2.07 (s, 2H, CH₂), 2.52 (s, 3H, CH₃—Ph), 2.58 (s, 3H, CH₃—Ph), 2.08-2.80 (a series of m, 2H, CH₂CO), 3.0-3.2 (m, 2H, CH₂N), 3.64 (s, 3H, CH₃O), 3.96-4.10 (m, 1H, CHN), 4.20-4.28 (m, 1H, CH), 4.28-4.40 (br. m, 2H, CH₂), 5.82-6.18 (m,1H, CHO), |
| | 7.24-7.80 (a series of m, |
| | 18H, fulvene, and Ph), |
| | HPLC t_{R} = 6.53, |
| | (M⁺ + 1) = 930.56. |

Synthesis of (4-Fmoc-5-substituted-6-oxo-piperazin-2-yl)-acetic acid (37): A solution of 10 mmol of compound (41) in 30 mL of 30% diethylamine in ethyl acetate was stirred at room temperature for 3 hours. The solution was then concentrated to dryness, redissolved in 2×30 mL of ethyl acetate, and reconcentrated. The residue dissolved in 50 mL of ethanol, and 20 mL of 1N hydrochloric acid solution, and hydrogenated at room temperature and atmospheric pressure overnight, filtered through celite, and concentrated to dryness. The residue was dissolved in 20 mL of tetrahydrofuran, and 10 mL of water, and 2.52 g (30 mmol) of solid sodium bicarbonate was added, followed by the addition of 3.3 g (13 mmol) of Fmoc-Cl. The mixture was stirred for 3 hours, diluted with 100 mL of ethyl acetate, the layers separated, and the organic layer washed with 2×50 mL of water, dried over magnesium sulfate, and concentrated. The product was purified by silica gel column chromatography.

| R | Analytical Data for Compounds (37) |
|---|---|
| 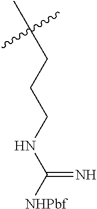 | ¹H NMR δ (CDCl₃) 1.2-1.6 (m, and s, 7H, CH₂, CH₃Ph), 2.10 (s, 2H, CH₂), 2.46 (s, 3H, CH₃—Ph), 2.56 (s, 3H, CH₃—Ph), 2.46-2.63 (br. m, 2H, CH₂CO), 3.0-3.95 (3 br. m, 5H, CH₂N, CHN), 4.10-4.30 (br. m, 1H, CH), 4.40-4.80 (br. m, 3H, CHN, CH₂), 7.22-7.80 (a series of m, 8H, fulvene), HPLC t_{R} = 5.73, (M⁺ + 1) 732.24. |

### Synthesis of Mimetics Employed in the Invention

The natriuretic peptide mimetics as disclosed in the several embodiments of this invention may be readily synthesized by any known conventional procedure for the formation of a peptide linkage between amino acids. Such conventional procedures include, for example, any solution phase procedure permitting a condensation between the free alpha amino group of an amino acid residue having its carboxyl group or other reactive groups protected and the free primary carboxyl group of another amino acid residue having its amino group or other reactive groups protected. In a preferred conventional procedure, the mimetics may be synthesized by solid-phase synthesis and purified according to methods known in the art. The amino acid surrogates of the present invention may be incorporated into mimetics by methods substantially similar to or identical to those employed with residues. Any of a number of well-known procedures utilizing a variety of resins and reagents may be used to prepare the natriuretic peptide mimetics.

The process for synthesizing the mimetics may be carried out by a procedure whereby each amino acid or amino acid surrogate in the desired sequence is added one at a time in succession to another amino acid residue or amino acid surrogate or by a procedure whereby peptide fragments with the desired amino acid sequence, which may include one or more amino acid surrogates, are first synthesized conventionally and then condensed to provide the desired mimetic. The resulting mimetic is cyclized to yield a cyclic mimetic of the invention.

Solid phase peptide synthesis methods are well known and practiced in the art. In such methods the synthesis of mimetics of the invention can be carried out by sequentially incorporating the desired amino acid residues or amino acid surrogates one at a time into the growing peptide chain according to the general principles of solid phase methods. These methods are disclosed in numerous references, including Merrifield R. B., Solid phase synthesis (Nobel lecture). Angew. Chem. 24:799-810 (1985) and Barany et al., The Peptides, Analysis, Synthesis and Biology, Vol. 2, Gross E. and Meienhofer J., Eds. Academic Press, 1-284 (1980).

In chemical syntheses of mimetics, reactive side chain groups of the various amino acid residues or amino acid surrogates are protected with suitable protecting groups, which prevent a chemical reaction from occurring at that site until the protecting group is removed. Also common is the protection of the alpha amino group of an amino acid residue or amino acid surrogate while that entity reacts at the carboxyl group, followed by the selective removal of the alpha amino protecting group to allow a subsequent reaction to take place at that site. Specific protecting groups have been disclosed and are known in solid phase synthesis methods and solution phase synthesis methods.

Alpha amino groups may be protected by a suitable protecting group, including a urethane-type protecting group, such as benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl. Fmoc is preferred for alpha amino protection.

Guanidino groups may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf), Fmoc and Boc. Pbf is one preferred protecting group for Arg. Other preferred protecting groups include Z, Fmoc, and Boc. It is to be understood that particularly guanidino protecting groups may be cleaved and removed during the synthetic process, or may alternatively not be cleaved or removed, in which event the side chain with the protecting group forms a derivative of an amino acid side chain moiety as defined herein. Particularly where the protecting group is labile, and may be removed by some mechanism in vivo upon administration to a patient, the mimetic becomes a “prodrug”, which is to say a mimetic that is a drug precursor which, following administration to a patient, is converted to the desired drug form in vivo via some chemical or physiological process (e.g., a prodrug on being brought to physiological pH or through enzyme action is converted to the desired drug form).

The natriuretic peptide mimetics utilized in the practice of this invention described herein can be prepared using solid phase synthesis, either manually or by means of an automated peptide synthesizer, using programming modules as provided by the manufacturer and following the protocols set forth by the manufacturer, or by modifications of the manufacturers's protocols to improve the yield of difficult couplings.

Solid phase synthesis is commenced from the C-terminal end of the mimetic by coupling a protected α-amino acid, α-amino acid surrogate or α-amino alcohol mimetic to a suitable resin. Such starting material is prepared by attaching an α-amino-protected amino acid or α-amino-protected amino acid surrogate by an ester linkage to a p-benzyloxybenzyl alcohol (Wang) resin or a 2-chlorotrityl chloride resin, by an amide bond between an Fmoc-Linker, such as p-[(R,S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art, such as by attaching an α-amino-protected alcohol mimetic to 3,4-dihydro-2H-pyran-2yl-methanol linker attached to chloromethyl polystyrene resin. Fmoc-Linker-BHA resin supports are commercially available and generally used when feasible. The resins are carried through repetitive cycles as necessary to add amino acids sequentially. The alpha amino Fmoc protecting groups are removed under basic conditions. Piperidine, piperazine, diethylamine, or morpholine (20-40% v/v) in N,N-dimethylformamide (DMF) may be used for this purpose.

Following removal of the alpha amino protecting group, the subsequent protected amino acids or amino acid surrogates are coupled stepwise in the desired order to obtain an intermediate, protected peptide-resin. The activating reagents used for coupling of the amino acids in the solid phase synthesis of the peptides are well known in the art. After the mimetic is synthesized, if desired, the orthogonally protected side chain protecting groups may be removed using methods well known in the art for further derivatization of the mimetic.

Reactive groups in a mimetic can be selectively modified, either during solid phase synthesis or after removal from the resin. For example, mimetics can be modified to obtain N-terminus modifications, such as acetylation, while on resin, or may be removed from the resin by use of a cleaving reagent and then modified. Methods for N-terminus modification, such as acetylation, or C-terminus modification, such as amidation or introduction of an N-acetyl group, are known in the art. Similarly, methods for modifying side chains of amino acids are well known to those skilled in the art of peptide synthesis. The choice of modifications made to reactive groups present on the mimetic will be determined, in part, by the characteristics that are desired in the mimetic.

The mimetic are, in one embodiment, cyclized prior to cleavage from the resin. For cyclization through reactive side chain moieties, the desired side chains are deprotected, and the mimetic suspended in a suitable solvent and a cyclic coupling agent added. Suitable solvents include, for example DMF, dichloromethane (DCM) or 1-methyl-2-pyrrolidone (NMP). Suitable cyclic coupling reagents include, for example, 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1(2H)-pyridyl)-1,1,3,3-tetramethyluronium tetrafluoroborate (TPTU) or N,N′-dicyclohexylcarbodiimide/1-hydroxybenzotriazole (DCCl/HOBt). Coupling is conventionally initiated by use of a suitable base, such as N,N-diispropylethylamine (DIPEA), sym-collidine or N-methylmorpholine (NMM).

Following cleavage of mimetics from the solid phase following synthesis, the mimetic can be purified by any number of methods, such as reverse phase high performance liquid chromatography (RP-HPLC), using a suitable column, such as a C₁₈ column. Other methods of separation or purification, such as methods based on the size or charge of the mimetic, can also be employed. Once purified, the mimetic can be characterized by any number of methods, such as high performance liquid chromatograph (HPLC), amino acid analysis, mass spectrometry, and the like.

Natriuretic peptide mimetics utilized in the practice of this invention with a substituted amide derivative C-terminus, typically an N-alkyl group, are prepared by solid phase synthesis commenced from the C-terminal end of the mimetic by coupling a protected alpha amino acid or amino acid surrogate to a suitable resin. Such methods for preparing substituted amide derivatives on solid phase have been described in the art. See, for example, Barn D. R., Morphy J. R., Rees D. C. Synthesis of an array of amides by aluminum chloride assisted cleavage of resin-bound esters. Tetrahedron Lett. 37, 3213-3216 (1996); DeGrado W. F. Kaiser E. T. Solid-phase synthesis of protected peptides on a polymer bound oxime: Preparation of segments comprising the sequences of a cytotoxic 26-peptide analogue. J. Org. Chem. 47:3258-3261 (1982). Such starting material can be prepared by attaching an alpha amino-protected amino acid or amino acid surrogate by an ester linkage to a p-benzyloxybenzyl alcohol (Wang) resin by well known means. The peptide chain is grown with the desired sequence of amino acids or amino acid surrogates, the product cyclized and resin-treated with a solution of appropriate amine and aluminum chloride (such as methyl amine, dimethyl amine, ethylamine, and so on) in dichloromethane. The resulting amide derivative mimetic is released in solution from the resin. The resin is filtered and the amide derivative mimetic recovered by concentration of solvent followed by precipitation with ether. The crude mimetic is dried and remaining amino acid side chain protective groups cleaved using trifluoroacetic acid (TFA) in the presence of water and triisopropylsilane (TIS). The final product is precipitated by adding cold ether and collected by filtration. Final purification is by RP-HPLC using a C₁₈ column.

In one preferred method, the natriuretic peptide mimetics of formula III are synthesized by the following methods. Each of the mimetics has one or two amino acid surrogates based on a keto-piperazine structure. The amino acid surrogates are synthesized as described above. The mimetics are synthesized using Fmoc chemistry. A manual synthetic approach is used for couplings immediately before and after incorporation of the keto-piperazine amino acid surrogate.

The following protocol was employed to attach an amino acid surrogate to resin, such as where the amino acid surrogate was in a terminal position. Rink amide resin (loading at 0.3 mmol/g, Advanced ChemTech) was allowed to swell in DMF for 30 minutes. Fmoc deprotection of the resin was accomplished using 20% piperidine/DMF for 20 minutes. Coupling of the resin with the selected Fmoc-protected keto-piperazine amino acid surrogate (2 eq) was accomplished by overnight incubation in DMF with PyBop (2 eq) and DIEA (4 eq). If following Kaiser testing a positive result was obtained, the coupling reaction was conducting a second time. Acetylation was carried out using Ac₂O (10 eq) and pyridine (20 eq) in DMF.

The following protocol was employed to attach a keto-piperazine amino acid surrogate to peptide-resin. Coupling was carried out by mixing Fmoc-protected keto piperzine amino acid surrogate (2 eq), TBTU (2 eq) and DIEA (4 eq) in DMF and allowing to incubate overnight, again with a repeat of the coupling reaction if a positive Kaiser test obtained. Acetylation was carried out using Ac₂O (10 eq) and pyridine (20 eq) in DMF.

The following protocol was employed to couple an Fmoc-protected amino acid to a keto-piperazine amino acid surrogate on solid phase. In most instances at least two coupling cycles were needed, and frequently three cycles were employed. In a typical cycle Fmoc-protected amino acid (4 eq) was mixed with HOAt (4 eq) and DIC (4 eq) in DMF. The resulting mixture was then mixed overnight in a SPE tube with a keto-piperazine amino acid surrogate attached directly or through intermediates to resin.

Couplings between amino acids that were not directly adjacent to a keto-piperazine amino acid surrogate in the sequence were conducted using standard protocols for solid phase peptide synthesis. The following protecting groups were employed: Boc for Lys and Orn, t-Butyl for Tyr and Ser, Trityl for Cys and His, O-t-Butyl for Asp and Pbf for Arg.

Mimetics were cleaved from resin employing a mixture of TFA/thioanisole/phenol/H₂O/DTT/TIS (87.5/2.5/2.5/5/2.5/11) (5 mL) for 3 hours. The resulting material was filtered and precipitated from cold ether under freezing conditions for one hour. Precipitated cysteinyl peptide was washed with cold ether at least three times before being use in an oxidation step.

For cyclization to form disulfide bonds via air oxidation, crude cysteinyl mimetic was dissolved in a mixture of acetonitrile and water. The pH of the reaction mixture was adjusted to 7-8 using 5% NH₄OH. The resulted solution was stirred slowly with 150 mg granular activated carbon for 2 days. Completion of cyclization was confirmed by LC-MS analysis before proceeding to the next process step. After cyclization, solid carbon was filtered from solution. The filtrate was lyophilized or dried in a speed-vac to obtain crude cyclic mimetic.

Certain mimetics of the invention, where the surrogate is bound to resin or other peptide solid support and is at the C-terminal position, may be synthesized by means of the following scheme. The following scheme is exemplified by synthesis of the mimetic of formula IV, but it is to be understood that substantially similar methods may be employed for any mimetic wherein the surrogate is bound to resin or other peptide solid support.

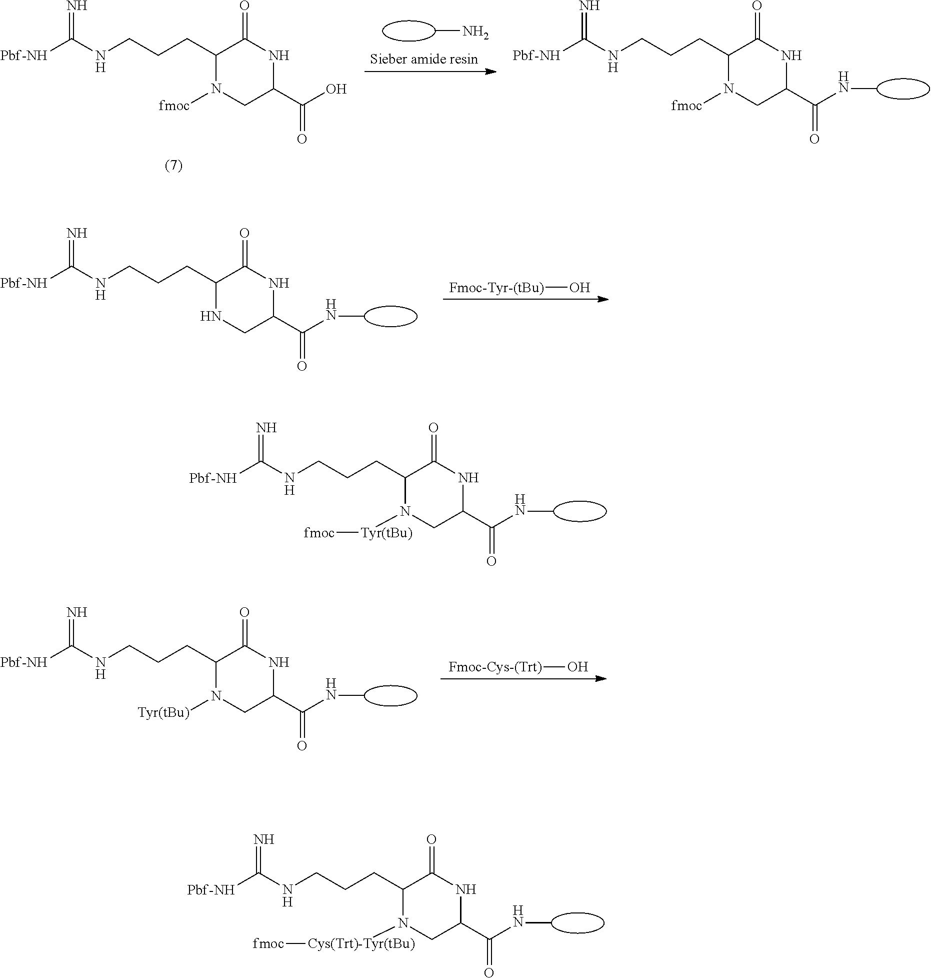

Surrogate (7) is prepared by the scheme of method A above, or any alternative method. Fmoc protected Sieber amide resin was treated by swelling 23.8 g (0.63 mmol/g substitution, 15 mmol) of the resin in 200 mL of a 1:1 mixture of dimethylformamide and dichloromethane for 45 minutes, followed by filtering and washing with 2×125 mL of dimethylformamide. The washed resin was then deprotected with 2×125 mL of 20% piperidine in dimethylformamide for 15 minutes, filtered, and washed with 4×125 mL of dimethylformamide.

A solution of 21.5 g (MW=717, 30 mmol) of Fmoc-protected surrogate (7) in 160 mL of dimethylformamide was added to the deprotected Sieber amide resin as prepared above, followed by 15.6 g (MW=520.3, 30 mmol) of solid PyBop, and 10.4 mL (MW=129.25, d=0.742, 60 mmol) of diisopropylethylamine, followed by another 40 mL of dimethylformamide. The mixture was agitated overnight with nitrogen bubbling. The resin was filtered, and washed with 4×130 mL of dimethylformamide, capped with 150 mL of capping solution consisting of a 3:2:1 solution of dimethylformamide:acetic anhydride:pyridine for 30 minutes, filtered, and washed with 4×130 mL of dimethylformamide to provide surrogate (7) complexed to resin.

The resulting Fmoc-protected surrogate (7) complexed to resin was deprotected with 2×130 mL of 20% piperidine in dimethylformamide for 15 minutes, filtered, and washed with 4×130 mL of dimethylformamide to yield surrogate (7) complexed to resin. A solution of 27.6 g of Fmoc-Tyr-(tBu)-OH (60 mmol, 4 eq.) in dimethylformamide (200 mL) was added to surrogate (7) complexed to resin, followed by a solution of 24.8 g of HCTU (60 mmol, 4 eq.), and 20.8 mL (120 mmol, 8 eq.) of diisopropylethylamine in DMF to a final volume of 200 mL and coupled overnight with nitrogen bubbling. The resulting Fmoc-Tyr-(tBu)-surrogate (7)-resin was isolated by filtration and washed with 2×130 mL of dimethylformamide. In order to ensure complete coupling, the product was again treated with a solution of 27.6 g of Fmoc-Tyr-(tBu)-OH (MW=459.6, 60 mmol, 4 eq.) in dimethylformamide to a final volume of 200 mL followed by a solution of 24.8 g of HCTU (60 mmol, 4 eq.), and diisopropylethylamine (20.8 mL, 120 mmol, 8 eq.) in DMF to a final volume of 200 mL and coupled overnight with nitrogen bubbling. The resin was filtered, and washed with 2×130 mL of dimethylformamide. HPLC and LC/MS showed that coupling between surrogate (7)-resin and Fmoc-Tyr-(tBu)-OH was complete.

The resulting Fmoc-Tyr-(tBu)-surrogate (7)-resin was then capped with 150 mL of capping solution as above for 30 minutes. The resin was then filtered, washed with 4×130 mL of dimethylformamide, 4×130 mL of dichloromethane, 2×130 mL of MeOH, 2×130 mL of diethyl ether, and dried under vacuum to give 36.7 g.

Thereafter each succeeding amino acid may be coupled. Before the coupling of the first amino acid, resulting Fmoc-Tyr-(tBu)-surrogate (7)-resin was swollen for 45 minutes with 200 mL of a 1:1 solution of dimethylformamide:dichloromethane. Each amino acid (Fmoc-AA-OH) was coupled by repeating the following cycle. The terminal amino acid residue was deprotected with 2×125 mL of 20% piperidine in dimethylformamide for 15 minutes, filtered and washed with 4×125 mL of dimethylformamide. The beads were checked by ninhydrin test. A solution of Fmoc-AA-OH (60 mmol, 4 eq.) in dimethylformamide to a final volume of 200 mL was added to resin, followed by a solution of HBTU (60 mmol, 4 eq.), and (120 mmol, 8 eq.) of N-methylmorpholine in DMF to a final volume of 200 mL [concentration of Fmoc-AA-OH=150 mM solution] and coupled for 30 minutes with nitrogen bubbling (coupling reaction checked by ninhydrin test). When the ninhydrin test was negative, the resin was filtered, and washed with 4×130 mL of dimethylformamide.

After all amino acids had been coupled, the resin was washed with 4×130 mL of dichloromethane, 4×130 mL of methanol, 4×130 mL of diethyl ether, and dried under vacuum to give product. The weight increase was quantitative.

100 mL of cleavage reagent consisting of a 81.5:5:5:5:2.5:1 solution of trifluoroacetic acid:phenol:thioanisole:water:DDT:triisopropyl silane was added to 32 g (˜6.4 mmol) of the following linear mimetic:

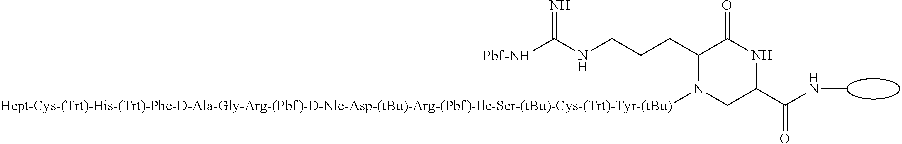

The suspension was allowed to stand at room temperature for 5 minutes and then filtered.

Another 100 mL of cleavage reagent was added to the resin, allowed to stand for 5 minutes, and filtered. This process was repeated.

The resulting resin was then washed with 2×40 mL of trifluoroacetic acid. The filtrates were combined and stirred for 2.5 hours at room temperature, and then concentrated under reduced pressure to ˜100 mL volume. Cold diethyl ether (1.5 L, pre-cooled to −20° C.) was added to the filtrate, and then placed in the freezer (−20° C.) for 1 hour, filtered through a sintered glass funnel, and the solids washed with 3×200 mL of cold diethyl ether, and then dried under vacuum for 1 hour with the solids triturated every 15 minutes to make sure solvent was removed efficiently. The following mimetic was obtained (15.4 g) (103% overall crude yield):

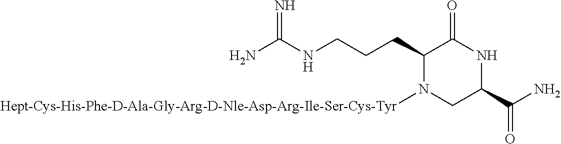

The above mimetic (15.4 g, 6.4 mmol) was dissolved in 16 L of 30% acetonitrile in water. The pH was adjusted to 8.4 using a solution of 5% ammonium hydroxide. Pulverized activated carbon (15.4 g) was added, and the suspension stirred overnight. The carbon was removed by filtration through celite. The celite was washed 3×100 mL 50% acetonitrile in water. The filtrates were combined, diluted with water to a final concentration of 10% acetonitrile, and loaded in the column for purification. Purification of the trifluoroacetate salt of the resulting mimetic was performed under the following conditions:

Column: Luna C₁₈, 10μ, 50×33 mm

Flow: 70 mL/minute

Solvent A: water containing 0.1% trifluoroacetic acid

Solvent B: acetonitrile containing 0.1% trifluoroacetic acid

Gradient: 5% solvent B for 5 minutes
26% B to 52% B in 30 minutes

The pure fractions were combined and lyophilized to give the purified trifluoroacetate salt of the mimetic. Dowex SBR, LCNG-OH resin (450 g) was suspended in 2 L of water, and gently stirred for 15 minutes, allowed to stand for 15 minutes, and then decanted. The procedure was repeated, and then 0.5 L of water added, and the slurry transferred into a 6×60 cm column. The water was drained, washed with 4 L of water, and ions exchanged with 6.5 L of 20% acetic acid solution. The resin was allowed to stand at room temperature overnight, and then washed with water until the pH of the filtrate was ˜4 (8 L of water used). The trifluoroacetate salt of the above mimetic (11.1 g), as prepared above, was dissolved in 80 mL of water, and loaded to the ion exchange resin, and eluted with water. Fractions containing 79-1 were combined, and 20% acetic acid solution was added to adjust the final concentration to 5% acetic acid, and then lyophilized. The natriuretic peptide mimetic of formula IV (10.4 g) was obtained:

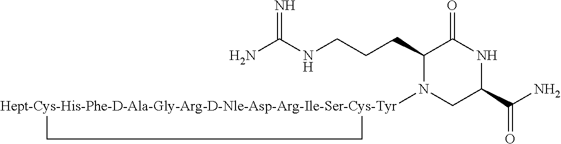

Similar methods may be employed with any mimetic where the surrogate is bound to resin or other peptide solid support and is at the C-terminal position.

Optional PEGylation of the peptide mimetics of the invention may be performed in any manner, such as those described below.

PEGylation of reactive amine groups, such as lysine or ornithine side chains, an omega amino aliphatic in position Aaa¹, or an amine group in J of an amino acid surrogate at Aaa¹⁵, was accomplished by dissolving 0.005 mmol purified mimetic in 2 mL of dimethylsulfoxide, followed by the addition of 55.5 mg (0.011 mmol, 2 eq) of PEG-5K-OSu (5,000 Da MW methoxy-PEG with a succinimidyl propionate reactive group), with 17.7 μL (0.13 mmol, 20 eq.) of triethyl amine then added, and the slightly cloudy solution stirred at room temperature for 3 hours. Excess PEG-5K-OSu was quenched by the addition of 7 μL (0.111 mmol, 10 eq.) of ethanol amine, and the reaction stirred overnight.

PEGylation of reactive carboxyl groups, such as Asp or Glu side chains or a terminal carboxyl at Aaa¹⁵ on either a residue or surrogate, is accomplished by coupling PEG-NH₂ (PEG-amine), to the mimetic containing a carboxylate group in the side chain of Asp or Glu or at the C-terminus. The peptide mimetic (0.005 mmol) is dissolved in DMSO (2 mL), followed by the addition of 55.5 mg (0.011 mmol, 2 eq) of PEG-NH₂ and HOBt (0.01 mmol). The coupling is started by the addition of 0.0055 mmole of coupling reagent N-ethyl-N′-(3-dimethylaminopropyl)-carbodiimide (EDAC). The slightly cloudy solution stirred at room temperature overnight. The PEGylated peptide mimetic is then purified by HPLC.

PEGylation of reactive thiol groups, such as Cys or Hcys side chains or a thiol group in Q of an amino acid surrogate at Aaa¹, is accomplished by treating the peptide mimetic in DMSO with PEG-methyl-maleimide reagent (SunBio, Orinda, Calif.) overnight. The PEGylated peptide mimetic is then purified by HPLC.

Following PEGylation, the resulting crude mixture was then purified by HPLC, yielding a PEG derivatized mimetic including one or more amino acid surrogates.

### In Vitro and In Vivo Test Systems

Selected natriuretic peptide mimetics were tested in assays to determine binding and functional status. The following assays were employed.

Cell Culture.

A cDNA clone that encodes for human natriuratic peptide receptor A (NPRA) was purchased from Bio S&T Inc. (Montreal, Quebec). The cDNA clone was inserted into the mammalian expression vector pcDNA3.1 (Invitrogen) and transfected into HEK-293 cells. Stable clones were selected by culture of cells in the presence of G418 sulfate. Expression of NPRA was examined by binding of [¹²⁵I]-atrial natriuretic peptide ([¹²⁵I]-ANP) to membrane homogenates prepared from clonal cell lines. HEK-hNPRA cells were maintained in culture at 37° C. in 5% CO₂ in Dulbecco's Modified Eagle's Medium (DMEM) supplemented with 10% FBS, G418 sulfate (300 μg/mL) sodium glutamate (0.29 mg/mL), penicillin (100 units/mL) and streptromycin (100 ug/mL).

Competitive Binding Assay.

A competitive inhibition binding assay was performed using crude membrane homogenates prepared from HEK-hNPRA cells. To prepare membrane homogenates, cells were rinsed with phosphate-buffered saline and incubated for 15 minutes at 4° C. in hypotonic lysis buffer (10 mM Tris, pH 7.4+5 mM EDTA). Cells were transferred from plates to polypropylene tubes and homogenized. Homogenates were centrifuged at 25,000×g for 20 minutes. Pellets were resuspended in buffer consisting of 50 mM Tris (pH 7.4) and 1 mM EDTA, homogenized and centrifuged at 25,000×g for 20 minutes. Pellets were resuspended in buffer consisting of 100 mM Tris (pH 7.4) and 10 mM MgCl₂ and stored at −80° C. until needed. On the day of an assay, homogenates were thawed and homogenized. Binding of [¹²⁵I]-ANP was carried out in buffer containing 25 mM Hepes (pH 7.4), 100 mM NaCl, 2 mM CaCl₂, 5 mM MgCl₂, 0.1% BSA and 1 mM 1,10-phenanthroline. Homogenates (1-10 μg protein/well) were incubated with [¹²⁵I]-ANP (25-30 μM) and increasing concentrations of competing ligands in Millipore filter plates for 120 minutes at 4° C. Assays were stopped by addition of cold wash buffer (phosphate-buffered saline) followed by filtration using a vacuum manifold. Bound radioactivity was determined using a gamma counter. Non-specific binding was defined by binding of [I¹²⁵]-hANP to non-transfected HEK293 membranes. Data were analyzed using GraphPad Prism® curve-fitting software.

General Method for Determination of EC₅₀.

Functional evaluation of natriuretic peptide mimetics was performed by measuring the accumulation of intracellular cGMP in HEK-293 cells that express recombinant hNPR-A. HEK-NPRA cells were harvested by washing and centrifugation in Cell Dissociation Buffer (Gibco, Life Technologies). Pelleted cells were resuspended in Hank's Balanced Salt Solution (HBSS) containing 10 mM Hepes (pH 7.4), 5 mM MgCl₂, 200 mM L-glutamine, 1 mM 1,10-phenanthroline and BSA (0.5 mg/mL). Following centrifugation, cells were resuspended in the above buffer supplemented with 0.5 mM 3-isobutyl-1-methylxanthine (IBMX). Cells (˜2×10⁵/well) were added to each well of a 96-well plate and incubated for 15 minutes at 37° C. Following the pre-incubation period, cells were incubated for an additional 15 minutes in the presence of increasing concentrations of mimetics. The reaction was terminated by lysis of the cells with temperature shock. The reaction plate was incubated in a dry ice/ethanol bath for 15 minutes followed by incubation at 90° C. for 10 minutes. Accumulation of cGMP was measured using the cGMP Flashplate RIA (Perkin-Elmer). Data analysis and EC₅₀ values were determined by using nonlinear regression analysis with GraphPad Prism® software.

Determination of Mass and Nuclear Magnetic Resonance Analysis.

The mass values of PEG-conjugated mimetics were analyzed by MALDI-TOF mass spectrometry (positive ion mode) using alpha-cyano-4-hydroxycinnamic acid (CHCA) as matrix. Methanol was used for sample preparation in mimetic to matrix ratios of 1:10, 1:20 and 1:30. Alternatively other matrices such as, sinapinic acid (SA) and 2, 5-dihydroxpenzoic acid (DHB), and solvents such acetonitrile—0.1% aqueous TFA can be used for sample preparation. Other determinations of mass values were made using a Waters MicroMass ZQ device utilizing a positive mode. For constructs that were not PEGylated, mass determinations were compared with calculated values and expressed in the form of mass weight plus two divided by two ((M+2)/2), unless otherwise specified.

Proton NMR data was obtained using a Bruker 300 MHz spectrometer. The spectra were obtained after dissolving mimetics in a deuterated solvent such as chloroform, DMSO, or methanol as appropriate.

HPLC measurements were made using a Waters Alliance HT with a YMC Pack Pro C₁₈ column (4.6×50 mm, 3μ) eluted at 1 mL/minute in a step-wise procedure. Solvent A (water containing 0.1% trifluoroacetic acid v/v) and solvent B (acetonitrile containing 0.1% trifluoroacetic acid v/v) were used as mobile phases. For analysis of keto piperazine intermediates, the column was equilibrated with 10% B and then B was increased to 90% over a period of 8 minutes. For analysis of peptides, the column was equilibrated with 2% B and then B was increased to 90% over a period of 8 minutes.

### Example 1

The mimetic of formula IV having the following structure was synthesized as described above:

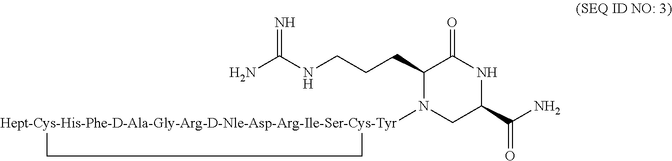

The resulting mimetic had formula of C₈₂H₁₂₇N₂₇O₂₀S₂ as the anhydrous, counter-ion free peptide and a molecular weight of 1875.22, determined as the average mass of the anhydrous, counter-ion free peptide. In the solid state form, the mimetic is a white to off-white solid with acetate as the associated counter-ion. The specific rotation of the mimetic as a 1.0% solution in water at 25° C. was found, after correction for mimetic content, to be:

[α]_{D}²⁵=−26.9°.

### Example 2

A formulation of the mimetic of formula IV was made for pharmaceutical use. The mimetic of formula IV was used in the acetate salt form. The formulation was dispensed into a vial which was stoppered and sealed, with each vial containing:
1 mg of the mimetic of formula IV, based on peptide weight net of acetate
1.181 mg succinic acid, NF
47.0 mg mannitol, USP
1N NaOH, USP, as needed to adjust pH
1N HCl, USP, as needed to adjust pH
Water for injection, to 1 mL volume
   The pH of the final product was adjusted to pH 5.75±0.05 with 1N NAOH or 1N HCl, as required. The resulting solution was filtered through a sterile 0.22 micron filter prior to vialing, and was stored at 5° C. until used.

An alternative formulation of the mimetic of formula IV was made for pharmaceutical use, similar to the formulation above, but additionally including between about 0.02 mg and 0.06 mg of disodium pamoate, such that the resulting solution was a pamoate suspension.

### Example 3

In a human clinical trial of a subcutaneously administered formulation in which the mimetic of formula IV was the active pharmaceutical ingredient, the half life of the mimetic of formula IV was determined to be approximately 3 hours. The mimetic of formula IV lead to a mean peak increase in plasma cGMP concentration of 1.7 ng/mL at a dose of 0.3 μg/kg of the mimetic of formula IV as a single, subcutaneous injection.

Cyclic guanosine monophosphate (cGMP) in the plasma samples was extracted using a protein precipitation method. cGMP and internal standard, cyclic adenosine monophosphate (cAMP), were separated using a HPLC technique and detected by an Applied Biosystems API-4000 liquid chromatography-tandem mass spectrometer (LC-MS/MS) with turbo-ion spray ionization (electrospray) in positive ion mode. Positive ions were detected in multiple reaction monitoring (MRM) mode with Precursor→Product ion pairs of 346.3→152.1 for cGMP, and 330.0→136.3 for cAMP.

### Example 4

Natriuretic peptide receptor binding affinity (expressed as Kᵢ) of the mimetic of formula IV (0.01 nM-1 μM) was evaluated using 3 human embryonic kidney (HEK) cell lines expressing recombinant human, dog, or rat NPRA. The Kᵢ values of the mimetic of formula IV for human, dog, and rat NPRA were 1, 41, and 10 nM, respectively (Table 1).

TABLE 1
Comparison of Affinity (Kᵢ) and Functional Potency (cGMP
Generation; EC₅₀ and Eₘₐₓ) of the Mimetic of Formula IV for
Human, Dog and Rat NPRA (Expressed in HEK Cell Lines).
(Results are given as Mean ± Standard Deviation)

| | | | Function (cGMP) | | |
|---|---|---|---|---|---|
| | | | | Eₘₐₓ | |
| | Binding | | | (% of ANP | |
| Receptor | Ki (nM) | n | EC₅₀ (nM) | response) | n |
| Human NPRA | 1 ± 1 | 45 | 2 ± 4 | 94 ± 8 | 51 |
| Dog NPRA | 41 ± 26 | 6 | 3 ± 1 | 94 ± 11 | 5 |
| Rat NPRA | 10 ± 3 | 4 | 14 ± 9 | 98 ± 7 | 47 |

As shown in Table 2, selectivity of the mimetic of formula IV for NPRA versus the other natriuretic peptide receptors, NPRB and NPRC, were determined in in binding studies in HEK cell lines. The mimetic of formula IV had a Kᵢ of 7±1 nM (n=4) for human NPRC, which is believed to be primarily a clearance receptor, and which is approximately a 7-fold lower affinity than for NPRA. The mimetic of formula IV was without effect (in concentrations up to 10 μM) in NPRB functional assays (cGMP generation; n=8).

TABLE 2
Comparison of Affinities (Kᵢ) and Functional Potencies (cGMP Generation;
EC₅₀ and Emax) of the mimetic of formula IV, hANP (human ANP)
and hBNP (human BNP) for hNPRA (human NPRA), hNPRB (human
NPRB) and hNPRC (human NPRC) (expressed in HEK cell lines).
(Results are given as Mean ± Standard Deviation)

| | hNPRA | | | | | hNPRB | | | hNPRC | |
|---|---|---|---|---|---|---|---|---|---|---|
| | Binding | | Function (cGMP) | | | Function (cGMP) | | | Binding | |
| Compound | Ki (nM) | n | EC₅₀ (nM) | Eₘₐₓ (%) | n | EC₅₀ (nM) | Eₘₐₓ (%) | n | Ki (nM) | n |
| Mimetic of | 1 ± 1 | 45 | 2 ± 4 | 94 ± 8 | 51 | Not | 1 ± 1 | 8 | 7 ± 1 | 4 |
| Formula IV | | | | | | calculable | | | | |
| hANP | 0.05 ± 0.2 | 6 | 0.4 ± 0.6 | 97 ± 14 | 54 | Not | 6 ± 2 | 5 | 0.05 ± 0.06 | 5 |
| | | | | | | calculable | | | | |
| hBNP | 3 ± 2 | 4 | 2 ± 2 | 96 ± 5 | 11 | Not | 9 ± 3 | 7 | 0.7 | 2 |
| | | | | | | calculable | | | | |

The natural natriuretic peptides, hANP and hBNP, have equivalent or higher affinity, respectively, for NPRC than NPRA, as compared to the mimetic of formula IV, which is 7-fold lower.

### Example 5

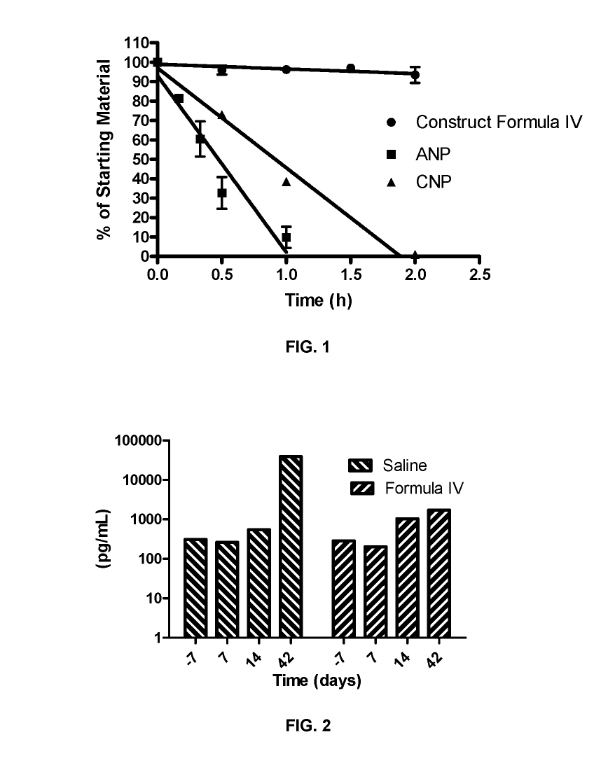

Endogenous natriuric peptides, such as hANP, hBNP and hCNP, are rapidly degraded by neutral endopeptidase (NEP). The reported human plasma half-life for hANP, hBNP and hCNP is about 2 minutes, 20 minutes and 2 minutes, respectively (Potter, L. R., Yoder, A. R., Flora, D. R., et al. “Natriuretic peptides: their structures, receptors, physiologic functions and therapeutic applications.” Handbook Exp. Pharmacol. 191:341-366 (2009)). A comparison was made between the sensitivity of the mimetic of formula IV and ANP to metabolism by human NEP (hNEP) (substrate concentration 50 μM). After two hours exposure to hNEP solution at 37° C., there was minimal degradation of the mimetic of formula IV (92% remaining; n=5). In contrast, under these conditions ANP was about 90% degraded after 60 minutes and 100% degraded after two hours (n=4). Similarly, for CNP there was 39% remaining after a 60 minute incubation with hNEP, and 1% after two hours (n=1). Results are shown in FIG. 1; results in FIG. 1 are expressed as the percent of the starting material, and are either the mean of 1-5 experiments or, where error bars are shown, are the mean±standard deviation.

### Example 6

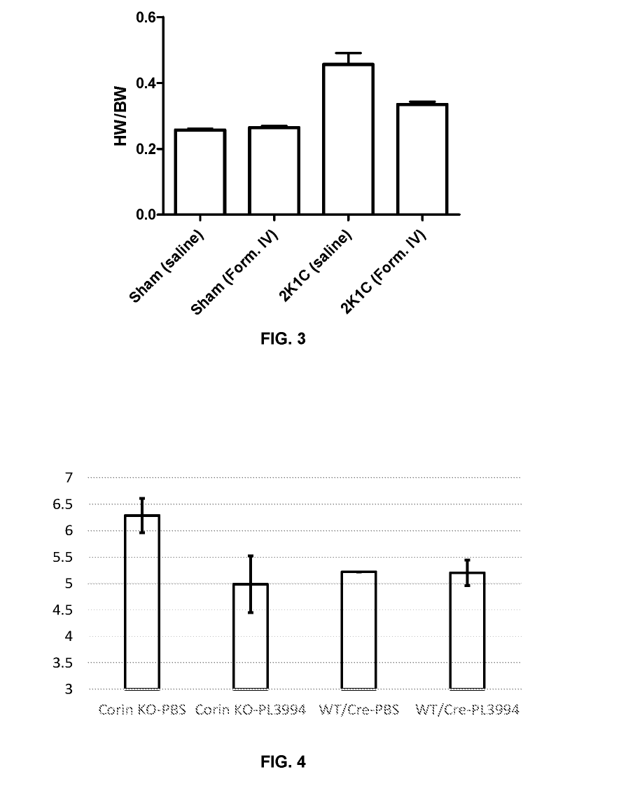

Rat studies were conducted using the two-kidney, one clip model, in which a clip was surgically placed around the left renal artery, which induced hypertension, increase in plasma aldosterone and cardiac hypertrophy. As shown in FIG. 2, by day 42 plasma aldosterone product was significant lower in animals receiving the mimetic of formula IV compared to animals receiving the saline control, where animals received 0.03 mg/kg of the mimetic of formula IV daily by subcutaneous injection. As show in FIG. 3, after 42 days animals receiving 0.03 mg/kg of the mimetic of formula IV daily by subcutaneous injection (2K1C (Form. IV)) had a significantly reduced heart weight to body weight ratio compared to animals receiving saline control (2K1C (saline)), while animals with a sham surgical procedure in which no clip was implanted had virtually identical heart weight to body weight ratios whether saline (Sham (saline)) or drug (Sham (Form. IV) was administered.

### Example 7

Use of the mimetic of formula IV in an experimental animal model of corin deficiency is evaluated. Corin conditional knockout mice, corin 623I(638P) knockin mice and wild-type mice are utilized, with moderate cardiac pressure overload induced by thoracic aortic banding, resulting in a 35-40 mmHg left ventricular pressure gradient. Conditional knockout mice receive injections to induce Cre-mediated, cardiomyocyte-restricted corin deficiency. Each group (wild-type control, conditional cardiomyocyte-restricted corin knockout and corin knockin mice) is divided into three subgroups, receiving daily placebo, low-dose mimetic of formula IV and high-dose mimetic of formula IV. Cardiac hypertrophy is measured by econcardiogram, changes in cardiac systolic and diastolic function, cardiac fibrosis, collagen turnover and other tests.

### Example 8

Corin knockout mice and wild-type control mice were implanted with an ALZET® osmotic infusion pump, and administered either control phosphate-buffered saline or mimetic of formula IV in phosphate-buffered saline in three different dose levels, 0.0001 mg/kg/min, 0.001 mg/kg/min or 0.01 mg/kg/min. Twenty-one days after implantation of the osmotic infusion pump, mice were sacrificed with avertin, and the hearts explanted, rinsed with phosphate-buffered saline and immediately flash frozen. Left ventricular apical tissue was homogenized, and both cGMP and protein kinase G serine/threonine kinase activity measured and normalized to 20 μg heart left ventricular tissue. A dose response was seen in both corin knockout mice and wild-type control mice, with the highest cGMP and relative protein kinase G serine/threonine kinase activity seen in mice receiving the 0.01 mg/kg/min dose of the mimetic of formula IV.

### Example 9

A pilot study was conducted in corin knockout mice to test the hypothesis that excessive hypertrophy seen in corin knockout mice subjected to thoracic aortic banding could be avoided or reduced by administration of the mimetic of formula IV. Corin knockout mice have deficient functional cardiac ANP caused by impaired corin-mediated natriuretic peptide pro-ANP pro-hormone activation cleavage. Male mice ten weeks of age, consisting of conditional cardiomyocyte restricted corin knockout mice and wild-type controls, had cardiac pressure overload induced by thoracic aortic banding. Mice were also implanted with an osmotic infusion pump as in Example 8, and administered either phosphate-buffered saline (control) or 0.01 mg/kg/min of the mimetic of formula IV. After two weeks, mice were sacrificed with avertin (tribromoethanol), and the hearts explanted, rinsed three times with phosphate-buffered saline and weighed, and then flash frozen for molecular studies. As shown in FIG. 4, corin knockout mice receiving control phosphate-buffered saline had a markedly higher heart weight/body weight (mg/g) ratio than did corin knockout mice receiving a 0.01 mg/kg/min dose of the mimetic of formula IV. The heart weight/body weight ratio in corin knockout mice receiving a 0.01 mg/kg/min dose of the mimetic of formula IV had similar ratios to wild-type controls receiving either phosphate-buffered saline or a 0.01 mg/kg/min dose of the mimetic of formula IV. In molecular studies using flash frozen hearts, left ventricular apical tissue was homogenized, and both cGMP and protein kinase G serine/threonine kinase activity measured and normalized to 20 μg heart left ventricular tissue. cGMP and relative protein kinase G serine/threonine kinase activity levels were higher in corin knockout mice receiving the 0.01 mg/kg/min dose of the mimetic of formula IV than in corin knockout mice receiving the phosphate-buffered saline control.

### Example 10

In a safety study, the mimetic of formula IV was administered to healthy volunteers. Doses of 0.1, 0.3, 0.7 and 1.0 μg/kg of the mimetic of formula IV were administered.

TABLE 3

| | Dose | | | | |
|---|---|---|---|---|---|
| Pharmacodynamic | 0.1 μg/kg | 0.3 μg/kg | 0.7 μg/kg | 1.0 μg/kg | Placebo |
| Parameters | Mean ± SD(N) | Mean ± SD(N) | Mean ± SD(N) | Mean ± SD(N) | Mean ± SD(N) |
| Emax (ng/mL) | 0.979 ± 0.456 | 1.83 ± 1.96 | 4.35 ± 1.02 | 4.33 ± 1.33 | 0.617 ± 0.017 |
| | (6) | (3) | (6) | (7) | (2) |
| AUEC(0-t) | 5.367 ± 5.612 | 0.6079 ± 14.94 | 10.30 ± 11.58 | 8.713 ± 23.32 | 2.246 ± 6.820 |
| (ng*hr/mL) | (6) | (3) | (6) | (7) | (2) |
| tmax (hr) median | 1.51 | 1.06 | 1.00 | 1.02 | 12.0 |
| (minimum, | (0.275, 6.01) | (0.999, 2.00) | (0.499, 2.01) | (0.251, 2.03) | (12.0, 12.0) |
| maximum) | (6) | (3) | (6) | (7) | (2) |
| t½ (hr) | NA | NA | 3.03 ± 0.345 | 2.98 ± 3.61 | NA |
| | (1) | (0) | (3) | (4) | (0) |
| Kel (1/hr) | NA | NA | 0.231 ± 0.025 | 1.05 ± 1.22 | NA |
| | (1) | (0) | (3) | (4) | (0) |

NA = Value missing or not reportable.

Kel is the apparent terminal elimination rate constant, calculated by linear regression of the terminal linear portion of the log concentration versus the time curve. The plasma cGMP concentrations returned to baseline levels by approximately 8 hours after dosing following 0.7 μg/kg and 1.0 μg/kg of the mimetic of formula IV.

### Example 11

A patient diagnosed with cardiovascular disease is tested for a level of functionally active corin in a biological sample of the patient. The test is either a genetic test for a genetic mutation, variation or polymorphism in the expression of corin, a test for levels of corin, or a test for mutations, variations or polymorphisms in the amino acid sequence of corin. The levels of functionally active corin are compared to a level of functionally active corin in a control, including a control comprising one or more samples from one or more healthy individuals or a reference standard. If the level of functionally active corin in the patient is less than a determined percentage of functionally active corin in the control, the patient is administered an NPRA agonist such as the NPRA agonist of formula IV.

### Example 12

A patient is tested for a functional active ANP₉₉₋₁₂₆ deficiency by genetic or protein testing. If the patient has a functional active ANP₉₉₋₁₂₆ deficiency, the patient is administered an NPRA agonist such as the NPRA agonist of formula IV.

### Example 13

A patient at risk for developing cardiovascular disease is tested for the level of functionally inactive corin in a biological sample of the patient. If a level of functionally inactive corin in the biological sample of the patient is detected, the patient is administered an NPRA agonist such as the NPRA agonist of formula IV.

### Example 14

A patient at risk for developing cardiovascular disease is tested for corin I555(P568) allele in a biological sample of the patient. If the patient is either heterozygous or homozygous for the corin I555(P568) allele, the patient is administered an NPRA agonist such as the NPRA agonist of formula IV.

### Example 15

A patient at risk for developing cardiovascular disease is tested to determine if the patient is either heterozygous or homozygous for a SNP in the ANP gene, including the SNP rs5058 or rs5065, in a biological sample of the patient. If the patient is either heterozygous or homozygous for a SNP in the ANP gene, the patient is administered an NPRA agonist such as the NPRA agonist of formula IV.

### Example 16

A patient with cardiac remodeling resulting from cardiovascular disease is tested for functional active ANP₉₉₋₁₂₆ deficiency by genetic or protein testing. If the patient has a functional active ANP₉₉₋₁₂₆ deficiency, the patient is administered an NPRA agonist such as the NPRA agonist of formula IV.

### Example 17

A patient diagnosed with cardiovascular disease is tested for a level of cAMP in a biological sample of the patient. The levels of cAMP are compared to a level of cAMP in a control, including a control comprising one or more samples from one or more healthy individuals or a reference standard. If the level of cAMP in the patient is less than a determined percentage of the the level of cAMP in the control, the patient is administered an NPRA agonist such as the NPRA agonist of formula IV.

### Example 18

A patient with cardiac remodeling resulting from cardiovascular disease is tested for functional active ANP₉₉₋₁₂₆ deficiency by genetic or protein testing. If the patient has a functional active ANP₉₉₋₁₂₆ deficiency, the patient is administered the NPRA agonist of formula IV by means of an implantable continuous or intermittent infusion device.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference.

What is claimed is:

1. A method of treating a patient with a functional atrial natriuretic peptide 99-126 (functional ANP) deficiency, comprising
administering a natriuretic peptide receptor A (NPRA) agonist in an amount sufficient to achieve a concentration of NPRA agonist together with endogenous active ANP₉₉₋₁₂₆, if any, of about 200 pg/mL to about 1,200 pg/mL in the serum or plasma.

2. The method of claim 1, wherein the concentration of NPRA agonist together with endogenous active ANP₉₉₋₁₂₆, if any, is about 400 pg/mL to about 800 pg/mL in the serum or plasma.

3. The method of claim 1, wherein administration comprises subcutaneous administration of the NPRA agonist.

4. The method of claim 3, wherein subcutaneous administration of the NPRA agonist comprises subcutaneous administration of a sustained release formulation.

5. The method of claim 3, wherein subcutaneous administration comprises subcutaneous infusion.

6. The method of claim 1, wherein the NPRA agonist is of formula IV: 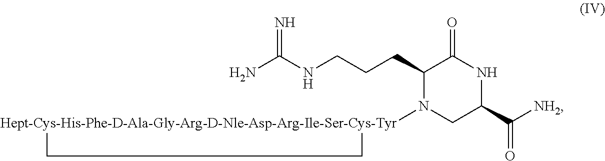
or a pharmaceutically acceptable salt of the NPRA agonist of formula IV.

7. The method of claim 6, wherein the NPRA agonist results in an increase in cyclic guanosine monophosphate (cGMP) in plasma.

8. The method of claim 7, wherein the increase in cGMP in plasma is an increase of about 0.4 to about 1.0 ng/mL.

9. The method of claim 6, wherein following administration of the NPRA agonist the area under the plasma concentration-time curve from time 0 to 24 hours, calculated using the linear trapezoidal rule (AUC₍₀₋₂₄₎) of NPRA agonist is at least about 0.50 ng·hr/mL.

10. The method of claim 9, wherein the (AUC₍₀₋₂₄₎) is at least about 0.75 ng·hr/mL.

11. A method of treating a patient with cardiovascular disease, comprising
detecting a level of functional ANP in the patient, wherein detecting a level of functional ANP in the patient comprises detecting a level of ANP₉₉₋₁₂₆ by detecting total atrial natriuretic peptide (total ANP) and pro-atrial natriuretic peptide (pro-ANP) in the patient, and subtracting the level of pro-ANP from total ANP;
comparing the level of functional ANP in the patient to a level of functional ANP in a control; and
administering an NPRA agonist to the patient if the level of functional ANP in the patient is less than the level of functional ANP in the control.

12. A method of treating a patient with cardiovascular disease, comprising
detecting a level of functional ANP in the patient;
detecting a level of pro-ANP in the patient;
comparing the ratio of functional ANP to pro-ANP in the patient to the ratio of functional ANP to pro-ANP in a control; and
administering an NPRA agonist to the patient if the ratio of functional ANP to pro-ANP in the patient compared to the ratio of functional ANP to pro-ANP in the control shows that the patient has a deficiency in functional ANP.

13. The method of claim 12, wherein detecting a level of functional ANP in the patient comprises detecting a level of ANP₉₉₋₁₂₆.

14. The method of claim 13, wherein detecting a level of ANP₉₉₋₁₂₆ comprises detecting total atrial natriuretic peptide and pro-atrial natriuretic peptide in the patient, and subtracting the level of pro-atrial natriuretic peptide from total atrial natriuretic peptide.

15. The method of claim 12, wherein the control comprises one or more samples from one or more healthy individuals, a reference standard or a combination thereof.

16. The method of claim 12, wherein administration comprises subcutaneous administration of the NPRA agonist.

17. The method of claim 16, wherein subcutaneous administration of the NPRA agonist comprises subcutaneous administration of a sustained release formulation.

18. The method of claim 16, wherein subcutaneous administration comprises subcutaneous infusion.

19. The method of claim 12, wherein the NPRA agonist is of formula IV: 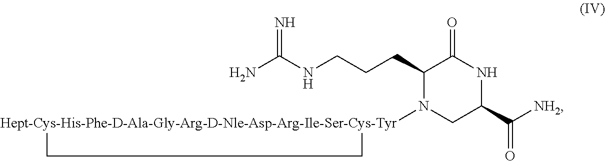
or a pharmaceutically acceptable salt of the NPRA agonist of formula IV.

20. The method of claim 19, wherein the NPRA agonist results in an increase in cGMP in plasma.

21. The method of claim 19, wherein following administration of the NPRA agonist the AUC₍₀₋₂₄₎ of NPRA agonist is at least about 0.50 ng·hr/mL.

22. A method for reducing cardiac remodeling in a patient with cardiovascular disease, comprising
detecting a level of functional ANP in the patient, wherein detecting a level of functional ANP in the patient comprises detecting a level of ANP₉₉₋₁₂₆, by detecting total atrial natriuretic peptide and pro-atrial natriuretic peptide in the patient, and subtracting the level of pro-atrial natriuretic peptide from total atrial natriuretic peptide;
comparing the level of functional ANP in the patient to a level of functional ANP in a control; and
administering an NPRA agonist to the patient if the level of functional ANP in the patient is less than the level of functional ANP in the control;
wherein the NPRA agonist is administered in an amount effective to alter the level of one or more parameters of cardiac remodeling by at least ten percent as compared to the levels of said one or more parameters prior to administering said composition, and wherein said one or more parameters are selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure.
